SEQUENCE LISTING (33 sequences)

SEQ ID NO: 1 - DNA, 1113 bp, Glycine Max
atggaagtagcacaggtactccacatgaacggtggcgttggagacgcaagctatgcaaacaactcccttgttcagcaaaaggtgatttgtttgacaaagcccataagagaggaagccataagaagcctctattgcagcacacaccccagaagcttggcaattgcagatttgggttgctcttctggaccaaacactttatttgttgtgtctgaattcataaaaattgtggagaagctttgccgagagctgaaccacaaatctccagaatacaaagtctttctgaatgatctccctgggaatgacttcaacaacatcttcaagtctcttgacagcgtcaaagagaaattgtgtgatgaaatggaaagtgggatcggtccatgctacttctcgggtgttcccggttctttctatggaagggttttcccatatcaaagtcttcattttgtccattcctcatacagccttcaatggctatctaaggttcctgagggtgtagacaacaacaagggcaatgtttacataggcagtacgagccccaaaaatgttgtgagagcttactatgagcaatttcagagagatttctctctttttctcaagtgtcgtgcagaggaattggttgaaggaggacgcatggttctcacatttttgggaagaagaagcgatgatccatctagcaaggatggttgctacatttgggagcttttggctactgctcttagtgatatggtcttgcagggaatcataagagaagagcaattagatacttttaacatccctcaatacactccatccccatctgaagtgaaattggaagttcttaaagaaggatcattcgccatcaatcgtctagaggtttctgaggtgaattggaatgctctcgatgagtggaatgctctagactttgaatctgaaaggtctgaatcacttagtgatggtgaatacaatgtggcacagtgcatgagggctgtggcagaacctatgctgattagccactttggtgaagctatcattgaagaggttttttgccgctaccagcaaatcttggctgaacgtatgtccaaggagaaaaccaagttcatcaatgttaccatattattgactagaaaagcataa

SEQ ID NO: 2 - DNA, 786 bp, Glycine Max
atgggttcacaaaattgtatggataggaagcactatgttctggtgcatggggcatgccatggagcttggtgttggtataagctcaagccacgcttggaatctgcaggccataaggtcacagtacttgaccttgcagcttctggaaccaacatgaagaaaattgaagatgttgatactttctcagagtattctgcgcctttgttgcagctaatggccacaattccctcaaatgagaagttagttctagttggtcacagccttggagggctgaacatagcacttgcaatggagaaattcccagaaaaggtagcagttggtgttttcttaacagcttttgctccagacactgaacaccacccatcttatgtcttggaaaagtacaatgagaggaccccgttagctgcatggttagacactgaatttgctccaagcggaaacaaaacatcaatgttcttcggccccaacttcttgtccgacaagctctaccaactgtcccctattgaggacttggaattggccaagactttagcaaggccatcatcgctcttcatggaagacttgactaaacaaaagaacttctccaaagagggatatgggtcagttccacgtgcctttattgtttgcacggaggaccttggaattccattggaatatcagctcttgatgatccaaaatgttgggttcaatgacgttgtagaggtcaaagacgcagatcatatggttatgctttgcaagccacaagaactattcgattccctccagcagatagcgactaaatatgcatga

SEQ ID NO: 3 - DNA, 15101 bp, Artificial sequence
  Synthetic plasmid condtruct pJL-OFP-35S::GmSAMT1
tcatggctctttcaaagcaaagtggggtcaaagatgtaatgaacaccgagcttcatatggacgggatcgtcaatggacacccctttgagataaaaggaaaaggaaagggaaacccgtacaagggtgtgcagaccatgaaacttacagtcattaagggtgcgcctttgccattttctattgacattttgctgcctcaacacatgtatggaagcaagccatttattaagtatcctgagagtatcccagactacatcaagttgtcatttcccgagggaatcacatgggaaaggtccatgacctttgaagatggtgcagtgtgcactgtctctaacgactccaggctcgatggcgactctttcatctacgaagtcaggtttcttggcgtgaactttccccgagatggacctgttatgcagaagaagacgcgaggctgggacccgtccacagagagactgtatgagtgtggtgggtggcagagaggagacgtccacatggccttgaagttggagaacggtggccattatacgtgcgacttcaaaactacttacaaatcaaagaagggcttgaaggtgccaccgtatcacttcgttgaccacaaactagatttactgagccacaacaccgatggtgctacctttgaagagtttgaacaacgagaaattgcacatgcacatctttctaacttaccggtagcccatcaccatcaccatcactaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatggctactactaagcatttggctcttgccatccttgtcctccttagcattggtatgaccaccagtgcaagaaccctcctagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtctgtacccgatcaacaccgagacccgtggcgtcttcgacctcaatggcgtctggaacttcaagctggactacgggaaaggactggaagagaagtggtacgaaagcaagctgaccgacactattagtatggccgtcccaagcagttacaatgacattggcgtgaccaaggaaatccgcaaccatatcggatatgtctggtacgaacgtgagttcacggtgccggcctatctgaaggatcagcgtatcgtgctccgcttcggctctgcaactcacaaagcaattgtctatgtcaatggtgagctggtcgtggagcacaagggcggattcctgccattcgaagcggaaatcaacaactcgctgcgtgatggcatgaatcgcgtcaccgtcgccgtggacaacatcctcgacgatagcaccctcccggtggggctgtacagcgagcgccacgaagagggcctcggaaaagtcattcgtaacaagccgaacttcgacttcttcaactatgcaggcctgcaccgtccggtgaaaatctacacgaccccgtttacgtacgtcgaggacatctcggttgtgaccgacttcaatggcccaaccgggactgtgacctatacggtggactttcaaggcaaagccgagaccgtgaaagtgtcggtcgtggatgaggaaggcaaagtggtcgcaagcaccgagggcctgagcggtaacgtggagattccgaatgtcatcctctgggaaccactgaacacgtatctctaccagatcaaagtggaactggtgaacgacggactgaccatcgatgtctatgaagagccgttcggcgtgcggaccgtggaagtcaacgacggcaagttcctcatcaacaacaaaccgttctacttcaagggctttggcaaacatgaggacactcctatcaacggccgtggctttaacgaagcgagcaatgtgatggatttcaatatcctcaaatggatcggcgccaacagcttccggaccgcacactatccgtactctgaagagttgatgcgtcttgcggatcgcgagggtctggtcgtgatcgacgagactccggcagttggcgtgcacctcaacttcatggccaccacgggactcggcgaaggcagcgagcgcgtcagtacctgggagaagattcggacgtttgagcaccatcaagacgttctccgtgaactggtgtctcgtgacaagaaccatccaagcgtcgtgatgtggagcatcgccaacgaggcggcgactgaggaagagggcgcgtacgagtacttcaagccgttggtggagctgaccaaggaactcgacccacagaagcgtccggtcacgatcgtgctgtttgtgatggctaccccggagacggacaaagtcgccgaactgattgacgtcatcgcgctcaatcgctataacggatggtacttcgatggcggtgatctcgaagcggccaaagtccatctccgccaggaatttcacgcgtggaacaagcgttgcccaggaaagccgatcatgatcactgagtacggcgcagacaccgttgcgggctttcacgacattgatccagtgatgttcaccgaggaatatcaagtcgagtactaccaggcgaaccacgtcgtgttcgatgagtttgagaacttcgtgggtgagcaagcgtggaacttcgcggacttcgcgacctctcagggcgtgatgcgcgtccaaggaaacaagaagggcgtgttcactcgtgaccgcaagccgaagctcgccgcgcacgtctttcgcgagcgctggaccaacattccagatttcggctacaagaacgctagccatcaccatcaccatcacgtgtgaattggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttatgcttttctagtcaataatatggtaacattgatgaacttggttttctccttggacatacgttcagccaagatttgctggtagcggcaaaaaacctcttcaatgatagcttcaccaaagtggctaatcagcataggttctgccacagccctcatgcactgtgccacattgtatccaccatcactaagtgattcagacctttcagattcaaagtctagagcattccactcatcgagagcattccaattcacctcagaaacctctagacgattgatggcgaatgatccttctttaagaacttccaatttcacttcagatggggatggagtgtattgagggatgttaaaagtatctaattgctcttctcttatgattccctgcaagaccatatcactaagagcagtagccaaaagctcccaaatgtagcaaccatccttgctagatggatcatcgcttcttcttcccaaaaatgtgagaaccatgcgtcctccttcaaccaattcctctgcacgacacttgagaaaaagagagaaatctctctgaaattgctcatagtaagctctcacaacatttttggggctcgtactgcctatgtaaacattgcccttgttgttgtctacaccctcaggaaccttagatagccattgaaggctgtatgaggaatggacaaaatgaagactttgatatgggaaaacccttccatagaaagaaccgggaacacccgagaagtagcatggaccgatcccactttccatttcatcacacaatttctctttgaagctgtcaagagacttgaagatgttgttgaagtcattcccagggagatcattcagaaagactttgtattctggagatttgtggttcagctctcggcaaagcttctccacaatttttatgaattcagacacaacaaataaagtgtttggtccagaagagcaacccaaatctgcaattgccaagcttctggggtgtgtgctgcaatagaggcttcttatggcttcctctcttatgggctttgtcaaacaaatcaccttttgctgaacaagggagttgtttgcatagcttgcgtctccaacgccaccgttcatgtggagtacctgtgctacttccatggatcctctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggctaatctggggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgac

SEQ ID NO: 4 - DNA, 14774 bp, Artificial sequence
  Synthetic plasmid condtruct pJL-OFP-35S::GmSABP2-1
tcatggctctttcaaagcaaagtggggtcaaagatgtaatgaacaccgagcttcatatggacgggatcgtcaatggacacccctttgagataaaaggaaaaggaaagggaaacccgtacaagggtgtgcagaccatgaaacttacagtcattaagggtgcgcctttgccattttctattgacattttgctgcctcaacacatgtatggaagcaagccatttattaagtatcctgagagtatcccagactacatcaagttgtcatttcccgagggaatcacatgggaaaggtccatgacctttgaagatggtgcagtgtgcactgtctctaacgactccaggctcgatggcgactctttcatctacgaagtcaggtttcttggcgtgaactttccccgagatggacctgttatgcagaagaagacgcgaggctgggacccgtccacagagagactgtatgagtgtggtgggtggcagagaggagacgtccacatggccttgaagttggagaacggtggccattatacgtgcgacttcaaaactacttacaaatcaaagaagggcttgaaggtgccaccgtatcacttcgttgaccacaaactagatttactgagccacaacaccgatggtgctacctttgaagagtttgaacaacgagaaattgcacatgcacatctttctaacttaccggtagcccatcaccatcaccatcactaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatggctactactaagcatttggctcttgccatccttgtcctccttagcattggtatgaccaccagtgcaagaaccctcctagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtctgtacccgatcaacaccgagacccgtggcgtcttcgacctcaatggcgtctggaacttcaagctggactacgggaaaggactggaagagaagtggtacgaaagcaagctgaccgacactattagtatggccgtcccaagcagttacaatgacattggcgtgaccaaggaaatccgcaaccatatcggatatgtctggtacgaacgtgagttcacggtgccggcctatctgaaggatcagcgtatcgtgctccgcttcggctctgcaactcacaaagcaattgtctatgtcaatggtgagctggtcgtggagcacaagggcggattcctgccattcgaagcggaaatcaacaactcgctgcgtgatggcatgaatcgcgtcaccgtcgccgtggacaacatcctcgacgatagcaccctcccggtggggctgtacagcgagcgccacgaagagggcctcggaaaagtcattcgtaacaagccgaacttcgacttcttcaactatgcaggcctgcaccgtccggtgaaaatctacacgaccccgtttacgtacgtcgaggacatctcggttgtgaccgacttcaatggcccaaccgggactgtgacctatacggtggactttcaaggcaaagccgagaccgtgaaagtgtcggtcgtggatgaggaaggcaaagtggtcgcaagcaccgagggcctgagcggtaacgtggagattccgaatgtcatcctctgggaaccactgaacacgtatctctaccagatcaaagtggaactggtgaacgacggactgaccatcgatgtctatgaagagccgttcggcgtgcggaccgtggaagtcaacgacggcaagttcctcatcaacaacaaaccgttctacttcaagggctttggcaaacatgaggacactcctatcaacggccgtggctttaacgaagcgagcaatgtgatggatttcaatatcctcaaatggatcggcgccaacagcttccggaccgcacactatccgtactctgaagagttgatgcgtcttgcggatcgcgagggtctggtcgtgatcgacgagactccggcagttggcgtgcacctcaacttcatggccaccacgggactcggcgaaggcagcgagcgcgtcagtacctgggagaagattcggacgtttgagcaccatcaagacgttctccgtgaactggtgtctcgtgacaagaaccatccaagcgtcgtgatgtggagcatcgccaacgaggcggcgactgaggaagagggcgcgtacgagtacttcaagccgttggtggagctgaccaaggaactcgacccacagaagcgtccggtcacgatcgtgctgtttgtgatggctaccccggagacggacaaagtcgccgaactgattgacgtcatcgcgctcaatcgctataacggatggtacttcgatggcggtgatctcgaagcggccaaagtccatctccgccaggaatttcacgcgtggaacaagcgttgcccaggaaagccgatcatgatcactgagtacggcgcagacaccgttgcgggctttcacgacattgatccagtgatgttcaccgaggaatatcaagtcgagtactaccaggcgaaccacgtcgtgttcgatgagtttgagaacttcgtgggtgagcaagcgtggaacttcgcggacttcgcgacctctcagggcgtgatgcgcgtccaaggaaacaagaagggcgtgttcactcgtgaccgcaagccgaagctcgccgcgcacgtctttcgcgagcgctggaccaacattccagatttcggctacaagaacgctagccatcaccatcaccatcacgtgtgaattggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctctcatgcatatttagtcgctatctgctggagggaatcgaatagttcttgtggcttgcaaagcataaccatatgatctgcgtctttgacctctacaacgtcattgaacccaacattttggatcatcaagagctgatattccaatggaattccaaggtcctccgtgcaaacaataaaggcacgtggaactgacccatatccctctttggagaagttcttttgtttagtcaagtcttccatgaagagcgatgatggccttgctaaagtcttggccaattccaagtcctcaataggggacagttggtagagcttgtcggacaagaagttggggccgaagaacattgatgttttgtttccgcttggagcaaattcagtgtctaaccatgcagctaacggggtcctctcattgtacttttccaagacataagatgggtggtgttcagtgtctggagcaaaagctgttaagaaaacaccaactgctaccttttctgggaatttctccattgcaagtgctatgttcagccctccaaggctgtgaccaactagaactaacttctcatttgagggaattgtggccattagctgcaacaaaggcgcagaatactctgagaaagtatcaacatcttcaattttcttcatgttggttccagaagctgcaaggtcaagtactgtgaccttatggcctgcagattccaagcgtggcttgagcttataccaacaccaagctccatggcatgccccatgcaccagaacatagtgcttcctatccatacaattttgtgaacccatggatcctctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggctaatctggggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgac

SEQ ID NO: 5 - DNA, 27 bp, Artificial sequence
  Synthetic oligonucleotide primer
atggaagtagcacaggtactccacatg

SEQ ID NO: 6 - DNA, 27 bp, Artificial sequence
  Synthetic oligonucleotide primer
tgcttttctagtcaataatatggtaac

SEQ ID NO: 7 - DNA, 27 bp, Artificial sequence
  Synthetic oligonucleotide primer
gtgaaattggaagttcttaaagaagga

SEQ ID NO: 8 - DNA, 26 bp, Artificial sequence
  Synthetic oligonucleotide primer
cagattcaaagtctagagcattccac

SEQ ID NO: 9 - DNA, 22 bp, Artificial sequence
  Synthetic oligonucleotide primer
gtgtaatgttggatgtgttccc

SEQ ID NO: 10 - DNA, 25 bp, Artificial sequence
  Synthetic oligonucleotide primer
acacaattgagttcaacacaaaccg

SEQ ID NO: 11 - DNA, 28 bp, Artificial sequence
  Synthetic oligonucleotide primer
aactatgctccccctggcaactatattg

SEQ ID NO: 12 - DNA, 30 bp, Artificial sequence
  Synthetic oligonucleotide primer
tctgaagtggtagcttctacatcgaaacaa

SEQ ID NO: 13 - DNA, 26 bp, Artificial sequence
  Synthetic oligonucleotide primer
aactacaattacgggcaagctggcaa

SEQ ID NO: 14 - DNA, 25 bp, Artificial sequence
  Synthetic oligonucleotide primer
ttgatggcttgtttccctgtgcagt

SEQ ID NO: 15 - DNA, 27 bp, Artificial sequence
  Synthetic oligonucleotide primer
atgggttcacaaaattgtatggatagg

SEQ ID NO: 16 - DNA, 27 bp, Artificial sequence
  Synthetic oligonucleotide primer
tcatgcatatttagtcgctatctgctg

SEQ ID NO: 17 - DNA, 255 bp, Artificial Sequence
  Synthetic oligonucleotide probe Gma.12911.1.A1_S_AT
tgccgctaccagcaaatcttggctgaacgtatgtccaaggagaaaaccaagttcatcaatgttaccatattattgactagaaaagcataagcaagagtttgcatctanatatggtttctcaaaatggattgttgtagttgttgtgagttgtgactgtttgcttgtttgtgtttttttctagcaattaactggcaaaatgttaagttggagaaaaagaaaactagtatgacgctgtacctttggtagtaagtttct

SEQ ID NO: 18 - DNA, 252 bp, Glycine max
agccgctaccaacaaattttggctgatcgtatgtctaaggagaaaactaagttcaccaatgtcaccatattattgactaaaaaggcataagaagtttgcatctattaatggtttctcaaattggattgttatggtaatgtggttggtgtgactgtttgcttgtttgtttgttttctagcaatgaactagcaaaatgttaagttggagaaaaaaaaaactagtatgatgttgtaccttttgtagtaactatta

SEQ ID NO: 19 - DNA, 257 bp, Glycine max
agccgctaccaacaaatcttgactgatcgtatgtccaaggagcaaactaagttcatcaatgtcaccgtattattgactagaaaagcataagcaagaagtttgcatctatatatggtttctcaaatggtattgctgtagtcatagttaaaaatccggttctactagtcagctcggatcggtccaattgagatctgatgatataacctttccgatttcgttattgaaccgatcatgcatttgatctggaatgacccggt

SEQ ID NO: 20 - DNA, 249 bp, Glycine max
agccgctaccaacaaatcttgactgatcgtatgtccaaggagcaaactaagttcatcaatgtcaccgtattattgactagaaaagcataagcaagaagtttgcatctatatagggtttctcaaatgggattgttgtagtcatactttaaaaatccggctcgactagcgtggtcggattgagatttgatgacataacctgtccgattttattattggaccgattatgtttttggtctagaataacccggt

SEQ ID NO: 21 - DNA, 233 bp, Glycine max
agccgttaccaacaaattttggctgatcgtatgtctaaggagaaaactaagttcaccaatgcaagaagtttgcatctatagcaaaacatagctggtcagagaaggcataatttctagttctattcactatccccttcctactactactctaaatttctccctttttgacatcatcaaaaaggaagaatctctaaccaggacaagctcgtatggggaatatagagctactagaa

SEQ ID NO: 22 - DNA, 255 bp, Glycine max
tgccgctaccagcaaatcttggctgaacgtatgtccaaggagaaaaccaagttcatcaatgttaccatattattgactagaaaagcataagcaagagtttgcatctatatatggtttctcaaaatggattgttgtagttgttgtgagttgtgactgtttgcttgtttgtgtttttttctagcaattaactggcaaaatgttaagttggagaaaaagaaaactagtatgacgctgtacctttggtagtaagtttct

SEQ ID NO: 23 - protein, 261 aa, Glycine Max
MGSQNCMDRKHYVLVHGACHGAWCWYKLKPRLESAGHKVTVLDLAASGTNMKKIEDVDTFSEYSAPLLQLMATIPSNEKLVLVGHSLGGLNIALAMEKFPEKVAVGVFLTAFAPDTEHHPSYVLEKYNERTPLAAWLDTEFAPSGNKTSMFFGPNFLSDKLYQLSPIEDLELAKTLARPSSLFMEDLTKQKNFSKEGYGSVPRAFIVCTEDLGIPLEYQLLMIQNVGFNDVVEVKDADHMVMLCKPQELFDSLQQIATKYA

SEQ ID NO: 24 - protein, 260 aa, Nicotiana tabacum
MKEGKHFVLVHGACHGGWSWYKLKPLLEAAGHKVTALDLAASGTDLRKIEELRTLYDYTLPLMELMESLSADEKVILVGHSLGGMNLGLAMEKYPQKIYAAVFLAAFMPDSVHNSSFVLEQYNERTPAENWLDTQFLPYGSPEEPLTSMFFGPKFLAHKLYQLCSPEDLALASSLVRPSSLFMEDLSKAKYFTDERFGSVKRVYIVCTEDKGIPEEFQRWQIDNIGVTEAIEIKGADHMAMLCEPQKLCASLLEIAHKYN

SEQ ID NO: 25 - protein, 256 aa, Arabidopsis thaliana
MKHYVLVHGGCHGAWCWYKVKPMLEHSGHRVTVFDLTAHGVNMSRVEDIQTLEDFAKPLLEVLESFGSDDKVVLVAHSLGGIPAALAADMFPSKISVAVFVTSFMPDTTNPPSYVFEKFLGSITEEERMDFELGSYGTDDHPLKTAFLGPNYLKNMYLLSPIEDYELAKMLMRVTPAITSNLTGTKSLTAQGYGSISRVYIVCGEDKGIRVDFQRWMIENSPVKEVMEIKDADHMPMFSKPHELCDRLLKIADKYP

SEQ ID NO: 26 - DNA, 20 bp, Artificial sequence
  Synthetic oligonucleotide primer
aattgaccaagagcttccgc

SEQ ID NO: 27 - DNA, 27 bp, Artificial sequence
  Synthetic oligonucleotide primer
ctacagaagcatcattttcaacatctt

SEQ ID NO: 28 - DNA, 21 bp, Artificial sequence
  Synthetic oligonucleotide primer
caattgaccaagagcttccaa

SEQ ID NO: 29 - DNA, 27 bp, Artificial sequence
  Synthetic oligonucleotide primer
ctatagaagcatcgttttcaacatctc

SEQ ID NO: 30 - DNA, 17 bp, Artificial sequence
  Synthetic oligonucleotide primer
ggccatttcggagctgg

SEQ ID NO: 31 - DNA, 24 bp, Artificial sequence
  Synthetic oligonucleotide primer
aggagaaggtggttctgtgagaga

SEQ ID NO: 32 - DNA, 20 bp, Artificial sequence
  Synthetic oligonucleotide primer
catggccatttcggagctta

SEQ ID NO: 33 - DNA, 22 bp, Artificial sequence
  Synthetic oligonucleotide primer
cagaagatggttctgcgaatgg

(19) United States
(12) Patent Application Publication
(10) Pub. No.: US 2014/0366219 A1
(43) Pub. Date: Dec. 11, 2014

(54) Increasing Soybean Defense Against Pests

(71) Applicant: University of Tennessee Research Foundation, Knoxville, TN (US)

(72) Inventors: C. Neal Stewart, Knoxville, TN (US); Jingyu Lin, Knoxville, TN (US); Mitra Mazarei, Knoxville, TN (US); Feng Chen, Knoxville, TN (US)

(21) Appl. No.: 14/369,565

(22) PCT Filed: Oct. 31, 2012
(86) PCT No.: PCT/US2012/062759

§ 371 Date: Jun. 27, 2014

Related U.S. Application Data
(60) Provisional application No. 61/581,039, filed on Dec. 28, 2011.

(51) Int. Cl. C12N 15/82

(52) U.S. Cl. CPC C12N 15/8286
USPC 800/279; 536/23.6; 435/320.1; 435/418; 800/302; 530/378; 554/9; 426/634

(57) ABSTRACT

Disclosed are pest resistance genes isolated from soybean. In several embodiments, a pest resistance gene isolated from soybean includes a nucleic acid sequence at least 80% identical to SEQ ID NO: 1 or SEQ ID NO: 2 or a degenerate variant thereof, or a fragment thereof. Also disclosed are expression vectors and constructs that include such nucleic acids. Methods are disclosed of producing a transgenic plant that has enhanced resistance to pests. Transgenic plants, plant cells or tissue (such as a dicotyledon or a monocotyledon plants, plant cells or tissue) transformed with the disclosed constructs are also disclosed.

### CROSS-REFERENCE TO RELATED APPLICATION

This application claims the priority benefit of U.S. Provisional Application 61/581,039 filed on Dec. 28, 2011, which is incorporated by reference herein in its entirety.

### FIELD OF THE DISCLOSURE

This disclosure concerns the field of plant molecular biology. More specifically, this disclosure pertains to tolerance to pathogens conferred by transgene expression of genes in soybeans and other plants.

### BACKGROUND

Soybean [Glycine max (L.) Merr.] is the most widely grown legume in the world, providing an important source of protein and oil. Soybean can be utilized in many ways such as ingredients in the formulation of a multitude of human foods, animal feed, and industrial products. As the dominant oil-seed in world trade, soybean contributes about 56% of global oilseed production. The estimated value of soybean products is about $48.6 billion world-wide and about $18.7 billion in the U.S. alone. Therefore, soybean is considered as one of the most important economic crops. Although global soybean production has increased steadily over the past century, future demand for soybean still cannot be satisfied due to a growing world population and limited land resources.

During its entire life cycle, soybean may be attacked by many pathogens, such as fungi, bacteria, viruses, and nematodes, and suffer many diseases in any tissue. For instance, the bacterial pathogen Pseudomonas syringae pv. glycinea, can cause bacterial blight, overwinter in crop residue, and be transmitted by seed. In laboratory studies, P. syringae is usually chosen as the model of bacterial pathogen. Fungal pathogens can also cause soybean diseases on both above- and below-ground tissues, accounting for approximately 50% of all soybean disease losses in the United States and around the world. Soybean mosaic virus (SMV) detected on seeds and on plants in the field, is the major virus causing soybean disease. Since this viral disease is seed-borne, it may cause problems in the next growing season. Its effect on seed quality is the main concern. Virus infected soybean may produce fewer, smaller, and often mottled seed. Another concern is that SMV infected soybean tends to be dually infected by other viruses, which magnifies the risk of yield loss and reduces seed quality even further. In addition, nematodes and soybean insect pests such as beetles, caterpillars, aphids, and spider mites can also cause yield loss of soybean.

The soybean cyst nematode (Heterodera glycines, SCN), is the pest that causes the most economic damage of soybean in the United States, causing nearly $1 billion in yield losses. SCN is a small plant-parasitic roundworm and most stages of SCN cannot be seen by unaided eyes. SCN feeds on the soybean roots and robs nutrients from the soybean. One important feature, genetic heterogeneity of SCN populations, endows this pathogen to readily overcome resistance. To complete their life cycle, infective second-stage juveniles (J2) enter host roots and migrate intracellularly within the cortical tissue to the vascular cylinder. The juveniles then initiate formation of specialized feeding sites called syncytia, which function as metabolic sinks to nourish the nematodes. The nematodes feed exclusively from their syncytia as they develop into adult males and females. Once fertilized, the female soybean nematode produces up to several hundred eggs that, for the most part, are retained within the nematode uterus. After the female's death, her body develops into a protective cyst around the eggs, giving the nematode its name.

SCN infestations are difficult to identify in the field because the above-ground symptoms of SCN infestations are easily confused with nutrient deficiency symptoms. When soybean plants are severely damaged by nematodes, they become stunted and turn chlorotic. These symptoms can be mistaken for nutrient deficiency and water stress. Until the population of SCN reaches a certain number, its damaging effect can not be clearly identified by farmers. Currently, the most accurate way of diagnosing SCN infection is to analyze a soil sample and observe the pest directly (Opperman and Bird 1998). Controlling SCN in commercial soybean productions still remains difficult because SCN has a short life cycle and populations can build rapidly. Frequent changes in population virulence of SCN also contribute to the difficulty in the management of this pest. In addition, the cysts of SCN can survive in the soil for up to nine years and then break to release the eggs under proper conditions, increasing the probability of the nematodes' dispersing via infested soil.

The methods used to control and manage SCN in soybean production include crop rotation, the use of SCN-resistant cultivars, and the application of nematicides, which are often used in an integrated manner. However, these approaches always face economic restrictions, are time-consuming, and use of nematicides can result in environmental problems. Additionally, for some areas, economic factors may limit the use of crop rotation. Considering the long-term demands of soybean, it is critical to develop resistant soybean cultivars through breeding for managing soybean diseases. Even for crop rotation, resistant soybean cultivars are also required. Thus, the need exists for the creation of plants, such as soybeans, that are pest resistance, such as resistant to SCN infestations. This disclosure meets that need.

### SUMMARY OF THE DISCLOSURE

Disclosed are previously unrecognized pest resistance genes isolated from soybean. In one embodiment, a pest resistance gene encodes a S-adenosyl-L-methionine (SAM) dependent carboxylmethyltransferase and is designated GmSAMT1. In another embodiment, a pest resistance gene encodes a methyl salicylate esterase and is designated GmSABP2-1.

In several embodiments, a pest resistance gene isolated from soybean includes a nucleic acid sequence at least 80% identical to SEQ ID NO: 1 or SEQ ID NO: 2 or a degenerate variant thereof, or a fragment thereof that encodes an active S-adenosyl-L-methionine (SAM) dependent carboxylmethyltransferase designated GmSAMT1 or methyl salicylate esterase designated GmSABP2-1. Isolated nucleic acids encoding such genes are disclosed as are proteins encoded by such genes. The isolated nucleic acids can be operably linked to promoter. Also disclosed are expression vectors and constructs that include such nucleic acids. In some embodiments an expression vector is pTh-OFP-35S::GmSAMT1 or pJL-OFP-35S::GmSABP2-1.

The constructs disclosed provide for enhanced resistance to pests, such as soybean cyst nematode infestation. Thus, methods are disclosed for producing a transgenic plant that has enhanced resistance to pests, such as soybean cyst nematode infestation. Transgenic plants, plant cells or tissue (such as a dicotyledon or a monocotyledon plants, plant cells or tissue) transformed with the disclosed constructs are also disclosed. Also provided is a plant seed, fruit, leaf, root, shoot, flower, cutting and other reproductive material useful in sexual or asexual propagation, progeny plants inclusive of F1 hybrids, male-sterile plants and all other plants and plant products derivable from the disclosed transgenic plant. Methods of producing the disclosed transgenic plants, plant cells or tissue are also provided herein.

The foregoing and other features and advantages will become more apparent from the following detailed description of several embodiments, which proceeds with reference to the accompanying figures.

### BRIEF DESCRIPTION OF THE DRAWINGS

FIGS. 1A-1C are a set of schematics showing a proposed model of soybean resistance against SCN and the functions of GmSAMT1 and GmSABP2-1. 1A shows the reactions between SA and MeSA that are catalyzed by GmSAMT1 and GmSABP2-1. SAM represents S-adenosyl-L-methionine functioning as the donor of a methyl group. 1B shows that in the resistant soybean root, the gene expression of GmSAMT1 and GmSABP2-1 is up-regulated when soybean is infected by SCN. The efficient transportation of mobile signal MeSA increases the soybean resistance against SCN. In the susceptible soybean root, the gene expression of GmSAMT1 does not change and the gene expression of GmSABP2-1 is down-regulated, when soybean is infected by SCN. The transportation of mobile signal MeSA is blocked, which decreases the soybean resistance against SCN. There are more SCNs completing their life cycle in the susceptible than the resistant soybean root. 1C shows that in SCN-infected local soybean root, SA is biosynthesized and the level of SA increases. The level of SA can be manipulated by GmSAMT1 through converting to mobile signal MeSA. The residual level of SA is sufficient for inducing GmPR-1 gene expression. The decreased level of SA benefits jasmonic acid (JA)-dependent defense pathway, which induces GmPR-3 gene expression. In undamaged soybean root, MeSA can be catalyzed by GmSABP2-1 to form SA, which plays a role of systemic acquired resistance against further SCN infection. Non-expressors of pathogenesis-related genes 1 (NPR1), WRKY family transcription factor, and lipoxygenase (LOX) may function as the regulatory points. The dots represent the adult female SCNs within soybean roots.

FIG. 2 shows the alignment of Affymetrix probe Gma.12911.1.A1_S_AT sequence (SEQ ID NO: 17) with five soybean candidate genes (SEQ ID NOS: 18-22). The sequence of Affymetrix probe Gma.12911.1.A1_S_AT aligned to the end of open reading frame sequence of the soybean genes and covers the part of 3′ UTR sequence (TAA in the box is the stop codon). Based on the alignment, only Glyma02g06070 has the identical sequence with the probe (indicated in the box).

FIG. 3 shows a phylogenetic tree of Glyma02g06070 and the functionally characterized SABATH family proteins from other plants. A neighbor joining tree based on the degree of sequence similarity between Glyma02g06070 and selected functionally characterized SABATH from other plant species using the ClustalX program. Glyma02g06070 is in the SAMT Glade shown in the oval. CbSAMT, Clarkia breweri SAMT (AF133053); AmSAMT, Antirrhinum majus (snapdragon) SAMT (AF515284); SfSAMT, Stephanotis floribunda SAMT (AJ308570); HcSAMT, Hoya carnosa SAMT (AJ863118); DwSAMT, Datura wrightii SAMT (EF472972); AmBAMT, Antirrhinum majus BAMT (AF198492); NsBSMT, Nicotiana suaveolens BSMT (AJ628349); AtBSMT, Arabidopsis thaliana BSMT (BT022049); AlBSMT, A. lyrata BSMT (AY224596); AbSAMT, Atropa belladonna SAMT (AB049752); OsBSMT1, Oryza sativa BSMT1 (XM467504); PhBSMT, Petunia hybrida BSMT (AY233465); OsIAMT1, O. sativa IAMT1 (EU375746); PtIAMT1, Populus trichocarpa IAMT1 (XP_{—}002298843); AtIAMT, A. thaliana IAMT (AK175586); AtGAMT1, A. thaliana GAMT1 (At4g26420); AtGAMT2, A. thaliana GAMT2 (At5g56300); CaCaS1, Coffea arabica caffeine synthase 1 (AB086414); CaXMT1, C. arabica XMT1 (AB048793); CaDXMT1, C. arabica DXMT1 (AB084125). Branches were drawn to scale with the bar indicating 0.1 substitutions per site.

FIG. 4 is a digital image of an SDS-PAGE gel of purified recombinant GmSAMT1 protein. His-tagged GmSAMT1 expressed in E. coli was purified as described in Example 1. Lane M contained protein molecular weight markers. Lane 1 contained crude extract and lane 2 contained about 2 μg of purified GmSAMT1 protein. The gel was stained with Coomassie blue.

FIGS. 5A-5D are set of graphs showing the biochemical properties of GmSAMT1. FIG. 5A shows the pH effect on GmSAMT 1 activity. The level of GmSAMT1 activity in 50 mM Tris-HCl buffer, pH 7.5, was arbitrarily set at 1.0. FIG. 5B shows the GmSAMT1 activity in response to temperature ranging from 0° C. to 60° C. The level of GmMAN1 activity at 25° C. was arbitrarily set at 1.0. FIG. 5C shows the effects of metal ions on activity of GmSAMT1. Metal ions were added to reactions in the form of chloride salts at 5 mM final concentrations. The level of GmSAMT 1 activity without any metal ion added as control (Ctr) was arbitrarily set at 1.0. FIG. 5D shows GmSAMT1 displayed pseudo Michaelis-Menton kinetics, with apparent Km value of 46.2±4.2 μM for salicylic acid.

FIG. 6 is a schematic showing the construction of pJL-OFP-35S::GmSAMT1, a plasmid for over-expression of GmSAMT1 gene in soybean roots. pJL-OFP was modified from pCAMBIA 1305.2 by inserting a 35S promoter-ppor RFP-NOS terminator at the Nod site. ppor RFP encodes a orange fluorescent protein. pJL-OFP is an Agrobacterium compatible binary plasmid for plant transformation and used for a vector control plasmid. pJL-OFP-35S::GUS is an intermediate vector and used for testing whether OFP and GUS within two cassettes co-express in soybean roots. pJL-OFP-35S::GmSAMT1 is obtained by replacing GUS gene with GmSAMT1 by digestion and ligation between the BamH I and Sac I sites.

FIGS. 7A and 7B are digital images showing a test for reporter gene expression in tobacco leaves and soybean hairy roots. FIG. 7A shows the transient expression of pJL-OFP and empty vector in tobacco leaves. FIG. 7B shows coexpression of two reporter genes in transgenic hairy roots. All the tested transgenic hairy roots with OFP signal showed GUS staining A schematic representation of the construct used for coexpression of an orange fluorescent protein (OFP) reporter gene and a GUS reporter gene is shown on the top. “35S-Pro” and “NOS-ter” represent the CaMV 35S promoter and the NOS terminator, respectively. Analysis of transgenic hairy roots transformed with the construct (top). OFP-positive transgenic hairy roots were separated from OFP-negative hairy roots under OFP filter. OFP-positive hairy roots were further demonstrated to be positive for GUS staining.

FIGS. 8A and 8B show the production of transgenic hairy roots of soybean overexpressing GmSAMT1. FIG. 8A. is a schematic representation of the construct used for coexpression of an orange fluorescent protein (OFP) reporter gene and GmSAMT1. “35S-Pro” and “NOS-ter” represent the CaMV 35S promoter and the NOS terminator, respectively. FIG. 8B. shows expression of GmSAMT1 in five types of transgenic hairy roots from three genetic backgrounds TN02-226, TN02-275 and Williams 82. “GmSAMT1” represents transgenic hairy roots containing GmSAMT1 transgene. “VC” represents transgenic hairy roots produced using a control vector containing OFP marker but not GmSAMT1 gene. qRT-PCR was performed with GmSAMT1-specific primers. Expression values were normalized to the expression levels of the soybean ubiquitin 3 gene (GmUBI3) in respective samples. The level of GmSAMT1 expression in the “VC” transgenic hairy roots of TN02-275 was arbitrarily set at 1.0. Each bar represents the mean relative expression level of three independent experiments with the standard errors, each containing a pool of five plants. The bars with asterisks are significantly different (LSD p<0.01).

FIGS. 9A and 9B are a set of bar graphs showing qRT-PCR analysis on transgenic hairy roots for GmPR-1, and GmPR-3. “GmSAMT1” represents transgenic hairy roots containing GmSAMT1 transgene. “VC” represents transgenic hairy roots produced using a control vector containing OFP marker but not GmSAMT1 gene. Each bar represents the mean relative expression level of three independent experiments with the standard errors. In the graphs shown in FIG. 9A and FIG. 9B, the bars with different letters are significantly different (LSD p<0.05).

FIG. 10 is a set of digital images showing the J2, J3, and J4 stages of soybean cyst nematodes shown in transgenic hairy roots by clearing and staining with acid fuchsin.

FIG. 11 is a bar graph and a set of digital images showing a SCN assay between transgenic hairy roots with over-expression of GmSAMT1 and control hairy roots. Each bar represents the mean of resistance index with the standard error from 10 soybean hairy roots. The resistance index is the ratio of the sum of J3 and J4 nematodes to total infecting female nematodes shown in A. rhizogenes-transformed hairy roots after two-week post inoculation by SCN. VS and VR represent the hairy root lines harboring plasmid pJL-OFP in the susceptible (TN02-275 and Williams 82) and resistant (TN02-226) background, which serve as susceptible and resistant control lines respectively. SAMT represents the hairy root line with overexpression of GmSAMT1 and OFP reporter gene in the susceptible background. Bars with asterisks are significantly different (LSD p<0.01). The bottom photograph showed the representative stages of SCN in the responding transgenic hairy root lines.

FIGS. 12A and 12B are bar graphs showing relative transcript abundance of GmICS1 (12A) and GmICS2 (12B) in transgenic hairy roots with or without SCN infection. Two types of transgenic hairy roots of the Williams 82 background were produced and analyzed. “GmSAMT1” represents transgenic hairy roots containing GmSAMT1 transgene. “VC” represents transgenic hairy roots produced using a control vector containing OFP marker gene but not GmSAMT1. “SCN” represents the administration of SCN infection. “Ctr” represents the uninfected control. qRT-PCR was performed with GmSAMT1-specific primers. Expression values were normalized to the expression levels of the soybean ubiquitin 3 gene (GmUBI3) in respective samples. The level of GmSAMT1 expression in the “VC” transgenic hairy roots without SCN infection (“Ctr”) was arbitrarily set at 1.0. Each bar represents the mean relative expression level of three independent experiments with the standard errors, each containing a pool of five plants. Bars with different letters are significantly different (LSD p<0.05).

FIGS. 13A and 13B are bar graphs showing relative transcript abundance of GmNPR1-1 (13A) and GmNPR1-2 (13B) in transgenic hairy roots with or without SCN infection. Two types of transgenic hairy roots of the Williams 82 background were produced and analyzed. “GmSAMT1” represents transgenic hairy roots containing GmSAMT1 transgene. “VC” represents transgenic hairy roots produced using a control vector containing OFP marker gene but not GmSAMT1. “SCN” represents the administration of SCN infection. “Ctr” represents the uninfected control. qRT-PCR was performed with GmSAMT1-specific primers. Expression values were normalized to the expression levels of the soybean ubiquitin 3 gene (GmUBI3) in respective samples. The level of GmSAMT1 expression in the “VC” transgenic hairy roots without SCN infection (“Ctr”) was arbitrarily set at 1.0. Each bar represents the mean relative expression level of three independent experiments with the standard errors, each containing a pool of five plants. Bars with different letters are significantly different (LSD p<0.05).

FIG. 14 is a bar graph showing qRT-PCR analysis on soybean mosaic virus infected soybean leaves for GmSAMT1 and GmPR-1. The grey and white bars represent the relative gene expression from the soybean plants with the resistant and susceptible responses, respectively. Each bar represents the mean relative expression level of three independent experiments with the standard errors. The bars with star(s) mean the gene expression are significantly different with the bars without star(s) (LSD p<0.05).

FIG. 15 shows a multiple sequence alignment of soybean Glyma16g26060 (SEQ ID NO: 23), tobacco SABP2 (NtSABP2) (SEQ ID NO: 24), and an Arabidopsis ortholog of SABP2 (At4g37150) (SEQ ID NO: 25). Identical residues are shaded in black and similar residues in gray. The catalytic triad residues are indicated by arrows, and residues that contact salicylic acid are indicated with triangles.

FIG. 16 is a digital image of an SDS-PAGE gel of purified recombinant GmSABP2-1 protein. His-tagged GmSABP2-1 expressed in E. coli was purified as described in Example 2. Lane M contained protein molecular weight markers. Lane 1 contained crude extract and lane 2 contained about 2 μg of purified GmSABP2-1 protein. The gel was stained with Coomassie blue.

FIG. 17 is a graph showing the calculation of the Km value of GmSABP2-1 using MeSA as substrate at concentrations of 5 μM to 100 μM. The apparent Km value was obtained through Lineweaver-Burk plots.

FIG. 18 is a graph showing the pH effect on GmSABP2-1 activity. Activity of the purified GmSABP2-1 was assayed from pH 6.0-9.0. The level of GmSABP2-1 activity in 50 mM Tris-HCl buffer, pH 7.0, was arbitrarily set at 1.0.

FIG. 19 is a bar graph showing a nematode demographic assay for SCN resistance between transgenic hairy roots with over-expression of GmSABP2-1 and control soybean lines. Ratio of the sum of J3 and J4 nematodes to the total infecting nematodes shown in hairy roots is used as the resistance index. The resistance index, WS, WR, VS and VR have been described. WS and WR are wild type hairy root lines in the susceptible and resistant background, respectively. VS and VR are hairy root lines harboring OFP reporter gene in the susceptible and resistant background, respectively as the vector control lines. SABP2 represents the hairy root line with over-expressed GmSABP2-1 and OFP reporter gene in TN02-275 background. Bars with different letters are significantly different (LSD p<0.05).

FIG. 20 shows a plasmid map of the pJL-OFP-35S::GmSAMT1 vector.

FIG. 21 shows a plasmid map of the pJL-OFP-35S::GmSABP2-1 vector.

### BRIEF DESCRIPTION OF THE SEQUENCE LISTING

The nucleic and amino acid sequences shown herein are shown using standard letter abbreviations for nucleotide bases and amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named UTK_{—}0141WP_ST25.txt, which was created on Oct. 30, 2012, is 56 kilobytes, and is incorporated by reference herein.

SEQ ID NO: 1 is an exemplary nucleic acid sequence of GmSAMT1.

SEQ ID NO: 2 is an exemplary nucleic acid sequence of GmSABP2-1.

SEQ ID NO: 3 is the nucleic acid sequence of pJL-OFP-35S::GmSAMT1.

SEQ ID NO: 4 is the nucleic acid sequence of pTh-OFP-35S::GmSABP2-1.

SEQ ID NOS: 5-16 and 26-33 are the nucleic acid sequences of primers.

SEQ ID NO: 17 is the nucleic acid sequence of Affymetrix probe Gma.12911.1.A1_S_AT.

SEQ ID NOS: 18-22 are the nucleic acid sequences of five soybean candidate genes.

SEQ ID NO: 23 is an amino acid sequence of Glyma16g26060.

SEQ ID NO: 24 is an amino acid sequence of tobacco SABP2

SEQ ID NO: 25 is an amino acid sequence of Arabidopsis ortholog of SABP2.

### DETAILED DESCRIPTION

### I. Introduction

Soybean cyst nematode (Heterodera glycines, SCN) is the most severe pest of soybean [Glycine max (L.) Merr.]. As disclosed herein. through microarray analysis on the gene expression of two genetically-related soybean lines (recombinant inbred lines, RILs) with different responses to SCN, the soybean genes Glyma02g06070 and Glyma16g26060 were identified to be SCN-induced in the resistant line but not in the susceptible line.

From sequence data, Glyma02g06070 was predicted to encode an 5-adenosyl-L-methionine (SAM) dependent carboxyl methyltransferase. Based on biochemical activity of Escherichia coli-expressed protein encoded by this gene, Glyma02g06070 was identified to be a salicylic acid methyltransferase gene and designated GmSAMT1. GmSAMT1 catalyzes the conversion of salicylic acid to methyl salicylate (MeSA). To determine the biological role of GmSAMT1 in soybean resistance against SCN, the inventors generated transgenic hairy roots in the susceptible soybean background with a binary vector harboring cassettes for the over-expression of GmSAMT1 and orange fluorescent protein (OFP) gene. After two weeks post inoculation by SCNs the demographics of SCN in over-expressed GmSAMT1 transgenic hairy roots and control hairy roots were compared. The result showed the transgenic hairy roots with over-expression of GmSAMT1 in the susceptible soybean line exhibited increased resistance, similar to that of the resistant soybean line. To further understand its defense role in soybean, the expression level of GmSAMT1 was also compared in the compatible and incompatible reactions between soybean and soybean mosaic virus. The expression of GmSAMT1 was found to be up-regulated in the incompatible reaction, and not the compatible reaction. Taken together, the results disclosed herein indicate that GmSAMT1 plays a critical role in soybean defense against SCN and soybean mosaic virus.

From sequence data, Glyma16g26060 was predicted to encode an α/β fold hydrolase. After cloning this gene and performing biochemical activity assay, this gene was identified to encode a methyl salicylate esterase. Since this gene was the ortholog gene of methyl salicylate esterase from tobacco, which was named NtSABP2, it was designated the soybean gene GmSABP2-1. The recombinant enzyme encoded by GmSABP2-1 can catalyze the conversion from MeSA to salicylic acid (SA). To explore the biological role of GmSABP2-1 in soybean resistance against SCN, the inventors generated transgenic hairy roots with over-expression of GmSABP2-1 and orange fluorescent protein (OFP) gene in the susceptible soybean background. After two weeks post inoculation by SCN eggs, the demographics of SCN in over-expressed GmSABP2-1 transgenic hairy roots and control hairy roots were compared. The results showed that the transgenic hairy roots with over-expressed GmSABP2-1 in the susceptible soybean line had increased resistance, which was similar to the resistant soybean control line. Therefore it was determined that GmSABP2-1 plays a role in soybean resistance against SCN through SA-dependent signaling pathway.

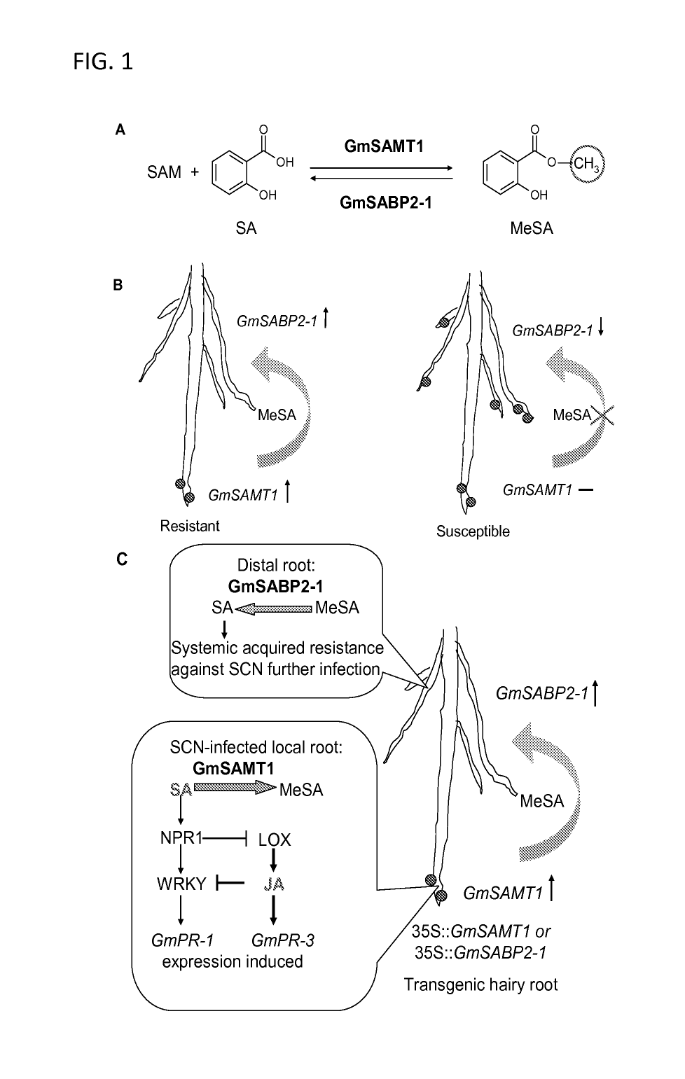

Taken together, the two components, GmSAMT1 and GmSABP2-1, work for the transportation of mobile signal MeSA and play an important role in soybean defense against SCN. While not being bound by theory, the functions of GmSAMT1 and GmSABP2-1 and a model of soybean resistance against SCN is proposed (see FIG. 1). In SCN-infected local root, GmSAMT1 plays a critical role of regulating the levels of basal SA and MeSA. In the root knot nematode resistance research, the residual level of free SA was found to be sufficient to confer basal resistance. This is indicates that the level of SA required for basal defense against SCN may be low. Biosynthesized SA can be changed to other forms, such as MeSA, to induce SAR in the distal root tissue. Relative high level of basal SA appear inhibit the activity of GmSABP2-1 and therefore inhibit the conversation of MeSA to SA at the local site. In the distal root tissue, the relative high level of MeSA and low level of SA favors the activity of GmSABP2-1, leading to the conversion of MeSA to SA. Thus, as disclosed herein through suitable specific and inducible promoters driving GmSAMT1 and GmSABP2-1 in transgenic plants soybean resistance can be achieved against SCN by manipulating SA and MeSA levels.

### II. Summary of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710).

The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will control.

To facilitate review of the various embodiments of this disclosure, the following explanations of terms are provided:

5′ and/or 3′: Nucleic acid molecules (such as, DNA and RNA) are said to have “5′ ends” and “3′ ends” because mononucleotides are reacted to make polynucleotides in a manner such that the 5′ phosphate of one mononucleotide pentose ring is attached to the 3′ oxygen of its neighbor in one direction via a phosphodiester linkage. Therefore, one end of a polynucleotide is referred to as the “5′ end” when its 5′ phosphate is not linked to the 3′ oxygen of a mononucleotide pentose ring. The other end of a polynucleotide is referred to as the “3′ end” when its 3′ oxygen is not linked to a 5′ phosphate of another mononucleotide pentose ring. Notwithstanding that a 5′ phosphate of one mononucleotide pentose ring is attached to the 3′ oxygen of its neighbor, an internal nucleic acid sequence also may be said to have 5′ and 3′ ends.

In either a linear or circular nucleic acid molecule, discrete internal elements are referred to as being “upstream” or 5′ of the “downstream” or 3′ elements. With regard to DNA, this terminology reflects that transcription proceeds in a 5′ to 3′ direction along a DNA strand. Promoter and enhancer elements, which direct transcription of a linked gene, are generally located 5′ or upstream of the coding region. However, enhancer elements can exert their effect even when located 3′ of the promoter element and the coding region. Transcription termination and polyadenylation signals are located 3′ or downstream of the coding region.

Agronomic trait: Characteristic of a plant, which characteristics include, but are not limited to, plant morphology, physiology, growth and development, yield, nutritional enhancement, disease or pest resistance, or environmental or chemical tolerance. An “enhanced agronomic trait” refers to a measurable improvement in an agronomic trait including, but not limited to, yield increase, including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. In some embodiments, an agronomic trait is resistance to a pest, such as soybean resistance to SCN.

Altering level of production or expression: Changing, either by increasing or decreasing, the level of production or expression of a nucleic acid molecule or an amino acid molecule (for example a gene, a polypeptide, a peptide), as compared to a control level of production or expression.

Amplification: When used in reference to a nucleic acid, this refers to techniques that increase the number of copies of a nucleic acid molecule in a sample or specimen. An example of amplification is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid. The product of in vitro amplification can be characterized by electrophoresis, restriction endonuclease cleavage patterns, oligonucleotide hybridization or ligation, and/or nucleic acid sequencing, using standard techniques. Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Cassette: A manipulable fragment of DNA carrying (and capable of expressing) one or more genes of interest between one or more sets of restriction sites. A cassette can be transferred from one DNA sequence (usually on a vector) to another by “cutting” the fragment out using restriction enzymes and “pasting” it back into the new context. In disclosed embodiments, a cassette includes or more of the disclosed inducible promoters.

cDNA (complementary DNA): A piece of DNA lacking internal, non-coding segments (introns) and transcriptional regulatory sequences. cDNA may also contain untranslated regions (UTRs) that are responsible for translational control in the corresponding RNA molecule. cDNA is usually synthesized in the laboratory by reverse transcription from messenger RNA extracted from cells or other samples. In some examples cDNA is used as a source of a gene of interest, such as a gene encoding GmSAMT1 or GmSABP2-1.

Construct: Any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more transcribable polynucleotide molecule has been operably linked.

Control plant: A plant that does not contain a recombinant DNA that confers (for instance) an enhanced or altered agronomic trait in a transgenic plant, is used as a baseline for comparison, for instance in order to identify an enhanced or altered agronomic trait in the transgenic plant. A suitable control plant may be a non-transgenic plant of the parental line used to generate a transgenic plant, or a plant that at least is non-transgenic for the particular trait under examination (that is, the control plant may have been engineered to contain other heterologous sequences or recombinant DNA molecules). Thus, a control plant may in some cases be a transgenic plant line that comprises an empty vector or marker gene, but does not contain the recombinant DNA, or does not contain all of the recombinant DNAs, in the test plant.

Degenerate variant and conservative variant: A polynucleotide encoding a polypeptide or an antibody that includes a sequence that is degenerate as a result of the genetic code. There are 20 natural amino acids, most of which are specified by more than one codon. Therefore, all degenerate nucleotide sequences are included as long as the amino acid sequence of the polypeptide encoded by the nucleotide sequence is unchanged. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified within a protein encoding sequence, the codon can be altered to any of the corresponding codons described without altering the encoded protein. Such nucleic acid variations are “silent variations,” which are one species of conservative variations. Each nucleic acid sequence herein that encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

Furthermore, one of ordinary skill will recognize that individual substitutions, deletions or additions which alter, add or delete a single amino acid or a small percentage of amino acids (for instance less than 5%, such as less than 4%, less than 3%, less than 2%, or even less than 1%) in an encoded sequence are conservative variations where the alterations result in the substitution of an amino acid with a chemically similar amino acid.

Conservative amino acid substitutions providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Not all residue positions within a protein will tolerate an otherwise “conservative” substitution. For instance, if an amino acid residue is essential for a function of the protein, even an otherwise conservative substitution may disrupt that activity.

Disease resistance or pest resistance: The avoidance of the harmful symptoms that are the outcome of the plant-pathogen interactions. Disease resistance and pest resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products.

As used herein, the term “pest” includes, but is not limited to, insects, fungi, bacteria, viruses, nematodes, mites, ticks, and the like. Insect pests include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Coleoptera, Lepidoptera, and Diptera. Viruses include but are not limited to tobacco or cucumber mosaic virus, ringspot virus, necrosis virus, maize dwarf mosaic virus, etc. Nematodes include but are not limited to parasitic nematodes such as root knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pallida (potato cyst nematodes). Lesion nematodes include but are not limited to Pratylenchus spp. Fungal pests include those that cause leaf, yellow, stripe and stem rusts. DNA (deoxyribonucleic acid): DNA is a long chain polymer which comprises the genetic material of most organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule includes the reverse complement of that DNA molecule. Except where single-strandedness is required by the text herein, DNA molecules, though written to depict only a single strand, encompass both strands of a double-stranded DNA molecule.

Encode: A polynucleotide is said to encode a polypeptide if, in its native state or when manipulated by methods known to those skilled in the art, the polynucleotide molecule can be transcribed and/or translated to produce a mRNA for and/or the polypeptide or a fragment thereof. The anti-sense strand is the complement of such a nucleic acid, and the encoding sequence can be deduced therefrom.

Enhancer domain: A cis-acting transcriptional regulatory element (a.k.a. cis-element) that confers an aspect of the overall control of gene expression. An enhancer domain may function to bind transcription factors, which are trans-acting protein factors that regulate transcription. Some enhancer domains bind more than one transcription factor, and transcription factors may interact with different affinities with more than one enhancer domain. Enhancer domains can be identified by a number of techniques, including deletion analysis (deleting one or more nucleotides from the 5′ end or internal to a promoter); DNA binding protein analysis using DNase I foot printing, methylation interference, electrophoresis mobility-shift assays, in vivo genomic foot printing by ligation-mediated PCR, and other conventional assays; or by DNA sequence comparison with known cis-element motifs using conventional DNA sequence comparison methods. The fine structure of an enhancer domain can be further studied by mutagenesis (or substitution) of one or more nucleotides or by other conventional methods. Enhancer domains can be obtained by chemical synthesis or by isolation from promoters that include such elements, and they can be synthesized with additional flanking nucleotides that contain useful restriction enzyme sites to facilitate subsequence manipulation.

(Gene) Expression: Transcription of a DNA molecule into a transcribed RNA molecule. More generally, gene expression encompasses the processes by which a gene's coded information is converted into the structures present and operating in the cell. Expressed genes include those that are transcribed into mRNA and then translated into protein and those that are transcribed into RNA but not translated into protein (for example, siRNA, transfer RNA and ribosomal RNA). Thus, expression of a target sequence, such as a gene or a promoter region of a gene, can result in the expression of an mRNA, a protein, or both. The expression of the target sequence can be inhibited or enhanced (decreased or increased). Gene expression may be described as related to temporal, spatial, developmental, or morphological qualities as well as quantitative or qualitative indications.

Gene regulatory activity: The ability of a polynucleotide to affect transcription or translation of an operably linked transcribable polynucleotide molecule, such as an inducible promoter. An isolated polynucleotide molecule having gene regulatory activity may provide temporal or spatial expression or modulate levels and rates of expression of the operably linked transcribable polynucleotide molecule. An isolated polynucleotide molecule having gene regulatory activity may include a promoter, intron, leader, or 3′ transcription termination region.

Genetic material: A phrase meant to include all genes, nucleic acid, DNA and RNA.

Heterologous nucleotide sequence: A sequence that is not naturally occurring with a promoter sequence. While this nucleotide sequence is heterologous to the promoter sequence, it may be homologous, or native, or heterologous, or foreign, to the plant host. The invention additionally encompasses expression of the homologous coding sequences of the promoters, particularly the coding sequences related to the resistance phenotype. The expression of the homologous coding sequences will alter the phenotype of the transformed plant or plant cell.

Increasing pest resistance or enhancing pest resistance: An enhanced or elevated resistance to a past over a normal or control plant or part thereof (for example a plant that has not been transformed with an isolated nucleic acid encoding S-adenosyl-L-methionine dependent carboxyl or methyl salicylate esterase, such as disclosed herein). In some examples, an increase or enhancement is an elevation of at least about 25%, 50%, 75%, 100%, 150%, 200%, 300%, 400%, 500% or more.

In cis: Indicates that two sequences are positioned on the same piece of RNA or DNA.

In trans: Indicates that two sequences are positioned on different pieces of RNA or DNA.

Insert DNA: Heterologous DNA within an expression cassettes, such as the disclosed expression cassette, used to transform the plant material while “flanking DNA” can comprise either genomic DNA naturally present in an organism such as a plant, or foreign (heterologous) DNA introduced via the transformation process which is extraneous to the original insert DNA molecule, e.g. fragments associated with the transformation event. A “flanking region” or “flanking sequence” as used herein refers to a sequence of at least 20, 50, 100, 200, 300, 400, 1000, 1500, 2000, 2500, or 5000 base pair or greater which is located either immediately upstream of and contiguous with or immediately downstream of and contiguous with the original foreign insert DNA molecule.

Isolated: An “isolated” biological component (such as a nucleic acid, peptide or protein) has been substantially separated, produced apart from, or purified away from other biological components in the cell of the organism in which the component naturally occurs, e.g., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Operably linked: This term refers to a juxtaposition of components, particularly nucleotide sequences, such that the normal function of the components can be performed. Thus, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame. A coding sequence that is “operably linked” to regulatory sequence(s) refers to a configuration of nucleotide sequences wherein the coding sequence can be expressed under the regulatory control (e.g., transcriptional and/or translational control) of the regulatory sequences.

Plant: Any plant and progeny thereof. The term also includes parts of plants, including seed, cuttings, tubers, fruit, flowers, etc. As used herein, the term plant includes plant cells, plant organs, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, stalks, roots, root tips, anthers, and the like. Progeny, variants, and mutants of the regenerated plants are also included within the scope of the invention. The term plant cell, as used herein, refers to the structural and physiological unit of plants, consisting of a protoplast and the surrounding cell wall, including those with genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed. The term plant organ, as used herein, refers to a distinct and visibly differentiated part of a plant, such as root, stem, leaf or embryo. More generally, the term plant tissue refers to any tissue of a plant in planta or in culture. This term includes a whole plant, plant cell, plant organ, protoplast, cell culture, or any group of plant cells organized into a structural and functional unit.

Polynucleotide molecule: Single- or double-stranded DNA or RNA of genomic or synthetic origin; that is, a polymer of deoxyribonucleotide or ribonucleotide bases, respectively, read from the 5′ (upstream) end to the 3′ (downstream) end.

Promoter: An array of nucleic acid control sequences which direct transcription of a nucleic acid, by recognition and binding of e.g., RNA polymerase II and other proteins (trans-acting transcription factors) to initiate transcription. A promoter includes necessary nucleic acid sequences near the start site of transcription, such as, in the case of a polymerase II type promoter, a TATA element. Minimally, a promoter typically includes at least an RNA polymerase binding site together with one or more transcription factor binding sites, which modulate transcription in response to occupation by transcription factors. Representative examples of promoters (and elements that can be assembled to produce a promoter) are described herein. Promoters may be defined by their temporal, spatial, or developmental expression pattern.

A plant promoter is a native or non-native promoter that is functional in plant cells. In one example, a promoter is a high level constitutive promoter, such as a tissue specific promoter.

Protein: A biological molecule, for example a polypeptide, expressed by a gene and comprised of amino acids.

Protoplast: An isolated plant cell without cell walls, having the potential for regeneration into cell culture or a whole plant.

Purified: The term purified does not require absolute purity; rather, it is intended as a relative term. Thus, for example, a purified protein preparation is one in which the protein is more enriched than the protein is in its generative environment, for instance within a cell or in a biochemical reaction chamber. Preferably, a preparation of protein is purified such that the protein represents at least 50% of the total protein content of the preparation.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one encoded for by a recombinant nucleic acid molecule.

Regulatable promoter or inducible promoter: A promoter the activity of which is regulated (directly or indirectly) by an agent, such as a transcription factor, a chemical compound, an environmental condition, or a nucleic acid molecule.

Regulating gene expression: Processes of controlling the expression of a gene by increasing or decreasing the expression, production, or activity of an agent that affects gene expression. The agent can be a protein, such as a transcription factor, or a nucleic acid molecule, such as a miRNA or an siRNA molecule, which when in contact with the gene or its upstream regulatory sequences, or a mRNA encoded by the gene, either increases or decreases gene expression.

Regulatory sequences or elements: These terms refer generally to a class of polynucleotide molecules (such as DNA molecules, having DNA sequences) that influence or control transcription or translation of an operably linked transcribable polynucleotide molecule, and thereby expression of genes. Included in the term are promoters, enhancers, leaders, introns, locus control regions, boundary elements/insulators, silencers, Matrix attachment regions (also referred to as scaffold attachment regions), repressor, transcriptional terminators (a.k.a. transcription termination regions), origins of replication, centromeres, and meiotic recombination hotspots. Promoters are sequences of DNA near the 5′ end of a gene that act as a binding site for RNA polymerase, and from which transcription is initiated. Enhancers are control elements that elevate the level of transcription from a promoter, usually independently of the enhancer's orientation or distance from the promoter. Locus control regions (LCRs) confer tissue-specific and temporally regulated expression to genes to which they are linked. LCRs function independently of their position in relation to the gene, but are copy-number dependent. It is believed that they function to open the nucleosome structure, so other factors can bind to the DNA. LCRs may also affect replication timing and origin usage. Insulators (also known as boundary elements) are DNA sequences that prevent the activation (or inactivation) of transcription of a gene, by blocking effects of surrounding chromatin. Silencers and repressors are control elements that suppress gene expression; they act on a gene independently of their orientation or distance from the gene. Matrix attachment regions (MARs), also known as scaffold attachment regions, are sequences within DNA that bind to the nuclear scaffold. They can affect transcription, possibly by separating chromosomes into regulatory domains. It is believed that MARs mediate higher-order, looped structures within chromosomes. Transcriptional terminators are regions within the gene vicinity that RNA polymerase is released from the template. Origins of replication are regions of the genome that, during DNA synthesis or replication phases of cell division, begin the replication process of DNA. Meiotic recombination hotspots are regions of the genome that recombine more frequently than the average during meiosis. Specific nucleotides within a regulatory region may serve multiple functions. For example, a specific nucleotide may be part of a promoter and participate in the binding of a transcriptional activator protein. Isolated regulatory elements that function in cells (for instance, in plants or plant cells) are useful for modifying plant phenotypes, for instance through genetic engineering.

RNA: A typically linear polymer of ribonucleic acid monomers, linked by phosphodiester bonds. Naturally occurring RNA molecules fall into three general classes, messenger (mRNA, which encodes proteins), ribosomal (rRNA, components of ribosomes), and transfer (tRNA, molecules responsible for transferring amino acid monomers to the ribosome during protein synthesis). Messenger RNA includes heteronuclear (hnRNA) and membrane-associated polysomal RNA (attached to the rough endoplasmic reticulum). Total RNA refers to a heterogeneous mixture of all types of RNA molecules.

Screenable Marker: A marker that confers a trait identified through observation or testing.

Selectable Marker: A marker that confers a trait that one can select for by chemical means, e.g., through the use of a selective agent (e.g., an herbicide, antibiotic, or the like). Selectable markers include but are not limited to antibiotic resistance genes, such as, kanamycin (nptII), G418, bleomycin, hygromycin, chloramphenicol, ampicillin, tetracycline, or the like. Additional selectable markers include a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulphonylurea resistance; or a methotrexate resistant DHFR gene. In one example, the selectable marker is AAD1.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Percent sequence identity is represented as the identity fraction multiplied by 100. The comparison of one or more polynucleotide or polypeptide sequences may be to a full-length polynucleotide or polypeptide sequence or a portion thereof, or to a longer polynucleotide sequence.

Methods of alignment of sequences for comparison are well known in the art. Various programs and alignment algorithms are described in: Smith and Waterman (Adv. Appl. Math. 2: 482, 1981); Needleman and Wunsch (J. Mol. Biol. 48: 443, 1970); Pearson and Lipman (PNAS. USA 85: 2444, 1988); Higgins and Sharp (Gene, 73: 237-244, 1988); Higgins and Sharp (CABIOS 5: 151-153, 1989); Corpet et al. (Nuc. Acids Res. 16: 10881-90, 1988); Huang et al. (Comp. Appls Biosci. 8: 155-65, 1992); and Pearson et al. (Methods in Molecular Biology 24: 307-31, 1994). Altschul et al. (Nature Genet., 6: 119-29, 1994) presents a detailed consideration of sequence alignment methods and homology calculations.

The alignment tools ALIGN (Myers and Miller, CABIOS 4:11-17, 1989) or LFASTA (Pearson and Lipman, 1988) may be used to perform sequence comparisons (Internet Program © 1996, W. R. Pearson and the University of Virginia, “fasta20u63” version 2.0u63, release date December 1996). ALIGN compares entire sequences against one another, while LFASTA compares regions of local similarity. These alignment tools and their respective tutorials are available on the Internet at with a web address of biology.ncsa.uiuc.edu.

Orthologs or paralogs (more generally, homologs) of a specified sequence are typically characterized by possession of greater than 75% sequence identity counted over the full-length alignment with the amino acid sequence of a specified protein (or the nucleic acid sequence of a specified nucleic acid molecule) using ALIGN set to default parameters. Sequences with even greater similarity to the reference sequences will show increasing percentage identities when assessed by this method, such as at least 80%, at least 85%, at least 90%, at least 92%, at least 95%, or at least 98% sequence identity. In such an instance, percentage identities will be essentially similar to those discussed for full-length sequence identity.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found in Sambrook et al. (In Molecular Cloning: A Laboratory Manual, Cold Spring Harbor, N.Y., 1989) and Tijssen (Laboratory Techniques in Biochemistry and Molecular Biology Part I, Ch. 2, Elsevier, New York, 1993). Nucleic acid molecules that hybridize under stringent conditions to a specified protein sequence will typically hybridize to a probe based on either the protein encoding sequence, an entire domain, or other selected portions of the encoding sequence under wash conditions of 0.2×SSC, 0.1% SDS at 65° C.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein. Substantial percent sequence identity is at least about 80% sequence identity, such as at least about 80%, at least about 85%, at least about 90%, at least about 95%, or even greater sequence identity, such as about 98% or about 99% sequence identity.

Soybean: Glycine max and includes all plant varieties that can be bred with soybean.

Soybean cyst nematode resistance gene: A gene that, when expressed in a plant contributes to resistance to soybean cyst nematode. Alternatively, a soybean cyst nematode gene may be an allelic variant of the resistance gene particularly variants resulting in a susceptible phenotype.

A transgenic event is produced by transformation of plant cells with a heterologous DNA construct(s), including a nucleic acid expression cassette that includes a transgene of interest, the regeneration of a population of plants resulting from the insertion of the transgene into the genome of the plant, and selection of a particular plant characterized by insertion into a particular genome location. In some embodiments of this disclosure, the transgene of interest is operable linked to a disclosed inducible promoter, such as SEQ ID NO: 1 or 2. An event is characterized phenotypically by the expression of the transgene(s). At the genetic level, an event is part of the genetic makeup of a plant. The term “event” also refers to progeny produced by a sexual outcross between the transformant and another variety that include the heterologous DNA. Even after repeated back-crossing to a recurrent parent, the inserted DNA and flanking DNA from the transformed parent is present in the progeny of the cross at the same chromosomal location. The term “event” also refers to DNA from the original transformant comprising the inserted DNA and flanking sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

Transgenic plant: A plant that contains a foreign (heterologous) nucleotide sequence inserted into either its nuclear genome or organellar genome.

Transgene: A nucleic acid sequence that is inserted into a host cell or host cells by a transformation technique.

Transgenic: This term refers to a plant/fungus/cell/other entity or organism that contains recombinant genetic material not normally found in entities of this type/species (that is, heterologous genetic material) and which has been introduced into the entity in question (or into progenitors of the entity) by human manipulation. Thus, a plant that is grown from a plant cell into which recombinant DNA is introduced by transformation (a transformed plant cell) is a transgenic plant, as are all offspring of that plant that contain the introduced transgene (whether produced sexually or asexually).

Transformation: Process by which exogenous DNA enters and changes a recipient cell. It may occur under natural conditions, or artificial conditions using various methods well known in the art. Transformation may rely on any known method for the insertion of foreign nucleic acid sequences into a prokaryotic or eukaryotic host cell. Selection of the method is influenced by the host cell being transformed and may include, but is not limited to, viral infection, electroporation, lipofection, and particle bombardment.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); and Ausubel et al., Short Protocols in Molecular Biology: A Compendium of Methods from Current Protocols in Molecular Biology, 4th ed., Wiley & Sons, 1999.

### III. Description of Several Embodiments

The present disclosure describes novel pest resistance genes, such a nucleic acid sequence encoding GmSAMT1 (such as set forth in SEQ ID NO: 1) and/or GmSABP2-1 such as set forth in SEQ ID NO: 2) or functional fragment thereof. Also provided are DNA constructs comprising a described pest resistance gene. In one embodiment, the pest resistance gene confers an agronomic trait to a plant in which it is expressed, for example pest resistance, such as resistance to SCN.

Also provided are transgenic plants. In one embodiment, a transgenic plant is stably transformed with a disclosed DNA construct. In some embodiments, the transgenic plant is a dicotyledon. In other embodiments, the transgenic plant is a monocotyledon. In one particular embodiment, the transgenic plant is a soybean plant. Further provided is a seed of a disclosed transgenic plant. In one embodiment, the seed comprises the disclosed DNA construct. Even further provided is a transgenic plant cell or tissue. In one embodiment, a transgenic plant cell or tissue comprises a disclosed pest resistance gene. In some embodiments, the plant cell or tissue is derived from a dicotyledon. In other embodiments, the plant cell or tissue is from a monocotyledon. In one particular embodiment, the plant cell or tissue is from a soybean plant.

Also provided are methods of producing a disclosed transgenic plant, plant cell, seed or tissue. In some embodiments, the method comprises transforming a plant cell or tissue with a disclosed DNA construct. In some embodiments, the method is a method of enhancing disease resistance in a plant.

Further provided are a plant cell, fruit, leaf, root, shoot, flower, seed, cutting and other reproductive material useful in sexual or asexual propagation, progeny plants inclusive of F1 hybrids, male-sterile plants and all other plants and plant products derivable from the disclosed transgenic plants.

A. Plant Pathogen Resistance Genes

The present disclosure provides previously unrecognized pest resistance genes isolated from soybean. In one embodiment, a pest resistance gene encodes a S-adenosyl-L-methionine (SAM) dependent carboxyl methyltransferase and is designated GmSAMT1. In another embodiment, a pest resistance gene encodes a methyl salicylate esterase and is designated GmSABP2-1.

In one embodiment, a GmSAMT1 nucleic acid sequence is provided as SEQ ID NO: 1 below:

(SEQ ID NO: 1)
ATGGAAGTAGCACAGGTACTCCACATGAACGGTGGCGTTGGAGA
CGCAAGCTATGCAAACAACTCCCTTGTTCAGCAAAAGGTGATTT
GTTTGACAAAGCCCATAAGAGAGGAAGCCATAAGAAGCCTCTAT
TGCAGCACACACCCCAGAAGCTTGGCAATTGCAGATTTGGGTTG
CTCTTCTGGACCAAACACTTTATTTGTTGTGTCTGAATTCATAA
AAATTGTGGAGAAGCTTTGCCGAGAGCTGAACCACAAATCTCCA
GAATACAAAGTCTTTCTGAATGATCTCCCTGGGAATGACTTCAA
CAACATCTTCAAGTCTCTTGACAGCGTCAAAGAGAAATTGTGTG
ATGAAATGGAAAGTGGGATCGGTCCATGCTACTTCTCGGGTGTT
CCCGGTTCTTTCTATGGAAGGGTTTTCCCATATCAAAGTCTTCA
TTTTGTCCATTCCTCATACAGCCTTCAATGGCTATCTAAGGTTC
CTGAGGGTGTAGACAACAACAAGGGCAATGTTTACATAGGCAGT
ACGAGCCCCAAAAATGTTGTGAGAGCTTACTATGAGCAATTTCA
GAGAGATTTCTCTCTTTTTCTCAAGTGTCGTGCAGAGGAATTGG
TTGAAGGAGGACGCATGGTTCTCACATTTTTGGGAAGAAGAAGC
GATGATCCATCTAGCAAGGATGGTTGCTACATTTGGGAGCTTTT
GGCTACTGCTCTTAGTGATATGGTCTTGCAGGGAATCATAAGAG
AAGAGCAATTAGATACTTTTAACATCCCTCAATACACTCCATCC
CCATCTGAAGTGAAATTGGAAGTTCTTAAAGAAGGATCATTCGC
CATCAATCGTCTAGAGGTTTCTGAGGTGAATTGGAATGCTCTCG
ATGAGTGGAATGCTCTAGACTTTGAATCTGAAAGGTCTGAATCA
CTTAGTGATGGTGAATACAATGTGGCACAGTGCATGAGGGCTGT
GGCAGAACCTATGCTGATTAGCCACTTTGGTGAAGCTATCATTG
AAGAGGTTTTTTGCCGCTACCAGCAAATCTTGGCTGAACGTATG
TCCAAGGAGAAAACCAAGTTCATCAATGTTACCATATTATTGAC
TAGAAAAGCATAA.

While a particular nucleic acid sequence has been shown for GmSAMT1, it is understood that a GmSAMT1 nucleic acid sequence includes any nucleic acid sequence redundant by virtue of the degeneracy of genetic code that encodes a GmSAMT1 protein, such as encoded by SEQ ID NO: 1.

In one embodiment, a GmSABP2-1 nucleic acid sequence is provided as SEQ ID NO: 2 below:

(SEQ ID NO: 2)
ATGGGTTCACAAAATTGTATGGATAGGAAGCACTATGTTCTGGT
GCATGGGGCATGCCATGGAGCTTGGTGTTGGTATAAGCTCAAGC
CACGCTTGGAATCTGCAGGCCATAAGGTCACAGTACTTGACCTT
GCAGCTTCTGGAACCAACATGAAGAAAATTGAAGATGTTGATAC
TTTCTCAGAGTATTCTGCGCCTTTGTTGCAGCTAATGGCCACAA
TTCCCTCAAATGAGAAGTTAGTTCTAGTTGGTCACAGCCTTGGA
GGGCTGAACATAGCACTTGCAATGGAGAAATTCCCAGAAAAGGT
AGCAGTTGGTGTTTTCTTAACAGCTTTTGCTCCAGACACTGAAC
ACCACCCATCTTATGTCTTGGAAAAGTACAATGAGAGGACCCCG
TTAGCTGCATGGTTAGACACTGAATTTGCTCCAAGCGGAAACAA
AACATCAATGTTCTTCGGCCCCAACTTCTTGTCCGACAAGCTCT
ACCAACTGTCCCCTATTGAGGACTTGGAATTGGCCAAGACTTTA
GCAAGGCCATCATCGCTCTTCATGGAAGACTTGACTAAACAAAA
GAACTTCTCCAAAGAGGGATATGGGTCAGTTCCACGTGCCTTTA
TTGTTTGCACGGAGGACCTTGGAATTCCATTGGAATATCAGCTC
TTGATGATCCAAAATGTTGGGTTCAATGACGTTGTAGAGGTCAA
AGACGCAGATCATATGGTTATGCTTTGCAAGCCACAAGAACTAT
TCGATTCCCTCCAGCAGATAGCGACTAAATATGCATGA.

While a particular nucleic acid sequence has been shown for GmSABP2-1, it is understood that a GmSABP2-1 nucleic acid sequence includes any nucleic acid sequence redundant by virtue of the degeneracy of genetic code that encodes a GmSABP2-1 protein, such as encoded by SEQ ID NO: 2.

Variants of the disclosed pest resistance genes isolated from soybean are also contemplated by this disclosure. Variant nucleotide sequences also include synthetically derived nucleotide sequences, such as those generated, for example, by using site-directed mutagenesis, but which when expressed still exhibit pest resistance activity. Methods for mutagenesis and nucleotide sequence alterations are well known in the art. See, for example, Kunkel (1985) Proc. Natl. Acad. Sci. USA 52:488-492; Kunkel et al. (1987) Methods in Enzymol. 75:367-382; U.S. Pat. No. 4,873,192; Walker and Gaastra, eds. (1983) Techniques in Molecular Biology (MacMillan Publishing Company, New York) and the references cited therein. It will further be understood that amino acid sequences encoded by GmSABP2-1 nucleic acids and GmSAMT1 nucleic acids will typically tolerate substitutions in the amino acid sequence and substantially retain biological activity. To routinely identify biologically active amino acid substitutions may be based on any characteristic known in the art, including the relative similarity or differences of the amino acid side-chain substituents, for example, their hydrophobicity, hydrophilicity, charge, size, and the like. Generally, nucleotide sequence variants will encode a protein have at least 80% sequence identity to the protein encoded by a disclosed pest resistance gene, such as at least at 80%, at least 85%, at least 90%, at least, 95% at least 96%, at least 97%, at least 98% at least 99% sequence identity or even greater to the protein encoded by its respective reference pest resistance gene nucleotide sequence. Thus, disclosed are GmSABP2-1 nucleic acids that encode a protein with about 80% sequence homology to the protein encoded by SEQ ID NO: 2 or a degenerate nucleic acid, such as at least at 80%, at least 85%, at least 90%, at least, 95% at least 96%, at least 97%, at least 98% at least 99% sequence identity or even greater to the protein encoded by SEQ ID NO: 2 or a degenerate nucleic acid. Also disclosed are GmSAMT1 nucleic acids that have about 80% sequence homology to the protein encoded by SEQ ID NO: 1 or a degenerate nucleic acid, such as at least at 80%, at least 85%, at least 90%, at least, 95% at least 96%, at least 97%, at least 98% at least 99% sequence identity or even greater to the protein encoded by SEQ ID NO: 1 or a degenerate nucleic acid. In some embodiments, a nucleic acid that have about 80% sequence homology to the protein encoded by SEQ ID NO: 1 or a degenerate nucleic acid, such as at least at 80%, at least 85%, at least 90%, at least, 95% at least 96%, at least 97%, at least 98% at least 99% sequence identity or even greater to the protein encoded by SEQ ID NO: 1 or a degenerate nucleic acid does not have an A at position number 627 relative to SEQ ID NO: 1. Also disclosed are GmSABP2-1 nucleic acids that have about 80% sequence homology to SEQ ID NO: 2 or a degenerate nucleic acid, such as at least at 80%, at least 85%, at least 90%, at least, 95% at least 96%, at least 97%, at least 98% at least 99% sequence identity or even greater to SEQ ID NO: 2 or a degenerate nucleic acid. Also disclosed are GmSAMT1 nucleic acids that have about 80% sequence homology to SEQ ID NO: 1 or a degenerate nucleic acid, such as at least at 80%, at least 85%, at least 90%, at least, 95% at least 96%, at least 97%, at least 98% at least 99% sequence identity or even greater to SEQ ID NO: 1 or a degenerate nucleic acid. In some embodiments, a nucleic acid that have about 80% sequence homology to SEQ ID NO: 1 or a degenerate nucleic acid, such as at least at 80%, at least 85%, at least 90%, at least, 95% at least 96%, at least 97%, at least 98% at least 99% sequence identity or even greater to SEQ ID NO: 1 or a degenerate nucleic acid does not have an A at position number 627 relative to SEQ ID NO: 1.

In some embodiments, a disclosed pest resistance nucleic acid encodes a functional fragment of a GmSABP2-1 or GmSAMT1 nucleic acids. Such functional fragments of the GmSABP2-1 and GmSAMT1 still exhibit pest resistance activity. Functional fragments include proteins in which residues at the N-terminus, C-terminus and/or internal to the full length protein have been deleted.

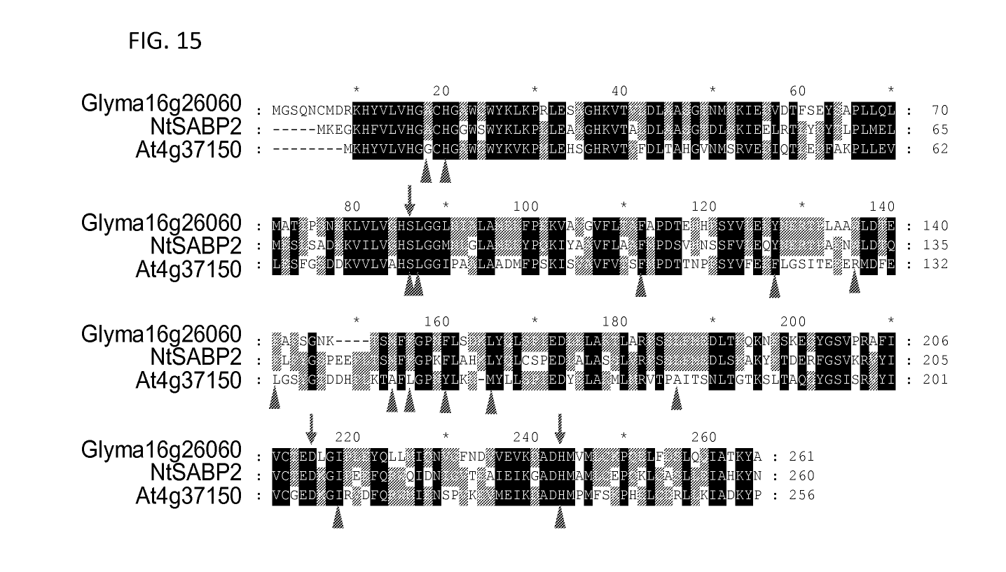

The protein encoded by the GmSABP2-1 gene is a member of the α/β hydrolase superfamily of enzymes, with Ser, Asp His catalytic triad, the location of which is shown by the arrows in FIG. 15. In addition, the crystal structure of the related enzyme from tobacco has been solved to atomic resolution (see Forouhar et al., PNAS 102 (5) 1773-1778, 2005). Thus, one of skill in the art can produce functional fragments and/or variants of gmSABP2 (and nucleic acids encoding such fragments) for example while maintaining the catalytic triad. Such fragments can be tested for activity with minimal experimentation given the guidance presented in the specification. For example based on the structure of the α/β hydrolase superfamily of enzymes, one of skill in the art can make deletions of a few amino acids on the N-terminus, C-terminus and/or internal loops of the protein encoded by the GmSABP2-1 gene without affecting the active site catalytic triad. For example a deletion of less than about 50, 40, 30, 25, 20, 15, 10, 5, 4, 3, 2, or 1 deletions from the N-terminus, C-terminus and/or internal loops while maintaining the active site catalytic triad with minimal testing and/or experimentation to determine the activity of the resultant protein.

The protein encoded by the GmSAMT1 gene is a S-adenosyl-L-methionine (SAM) dependent carboxylmethyltransferase designated GmSAMT1 belonging to the SABATH methyltransferases family. The crystal structure of members of the SABATH family have been solved to atomic resolution and the active site of the enzyme mapped (see Zhao et al. Plant Physiol. 146(2): 455-467, 2008). Thus, one of skill in the art can produce functional fragments and/or variants of GmSAMT1 (and nucleic acids encoding such fragments) for example while maintaining the catalytic triad. Such fragments can be tested for activity with minimal experimentation given the guidance presented in the specification. For example based on the structure of the SABATH family of enzymes, one of skill in the art can make deletions of a few amino acids on the N-terminus, C-terminus and/or internal loops of the protein encoded by the GmSAMT1 gene without affecting the active site c. For example a deletion of less than about 50, 40, 30, 25, 20, 15, 10, 5, 4, 3, 2, or 1 deletions from the N-terminus, C-terminus and/or internal loops while maintaining the active site catalytic triad with minimal testing and/or experimentation to determine the activity of the resultant protein.

B. Expression Cassettes

The nucleotide sequences for the disclosed pest resistance genes, such a nucleic acid sequence encoding GmSAMT1 (such as set forth in SEQ ID NO: 1 or a degenerate variant thereof) and/or GmSABP2-1 such as set forth in SEQ ID NO: 2 or a degenerate variant thereof) or functional fragment thereof, are useful in the genetic manipulation of any plant to confer resistance to pests, such as insects, fungi, bacteria, viruses, nematodes, mites, ticks, and the like, for example SCN when operably linked with a promoter, such as an indictable or constitutive promoter. By “operably linked” is intended the transcription or translation of the disclosed pest resistance genes is under the influence of the promoter sequence. In this manner, the nucleotide sequences for the disclosed pest resistance genes are provided in expression cassettes for expression in the plant of interest. Such expression cassettes will typically comprise a transcriptional initiation region comprising a promoter nucleotide sequence operably linked to one or more of the disclosed pest resistance genes or variants thereof. Such an expression cassette can be provided with a plurality of restriction sites for insertion of the nucleotide sequence to be under the transcriptional regulation of the regulatory regions. The expression cassette may additionally contain selectable marker genes or sequences. The expression cassettes of this disclosure can be part of and an expression vector, such as a plasmid.

In some embodiments, the transcriptional cassette will include in the 5′-to-3′ direction of transcription, a transcriptional and translational initiation region, a pest resistance gene, such a nucleic acid sequence encoding GmSAMT1 (such as set forth in SEQ ID NO: 1 or a degenerate variant thereof) and/or GmSABP2-1 such as set forth in SEQ ID NO: 2 or a degenerate variant thereof) or functional fragment thereof, and a transcriptional and translational termination region functional in plant cells. The termination region may be native with the transcriptional initiation region, may be native with the pest resistance gene, or may be derived from another source. Convenient termination regions are available from the Ti-plasmid of A. tumefaciens, such as the octopine synthase and nopaline synthase termination regions. See also, Guerineau et al., Mol. Gen. Genet. 262:141-144, 1991; Proudfoot Cell 64:671-674, 1991; Sanfacon et al., Genes Dev. 5:141-149, 1991; Mogen et al., Plant Cell 2:1261-1272, 1990; Munroe et al., Gene 91:151-158, 1990; Ballas et al., Nucleic Acids Res. 17:7891-7903, 1989; Joshi et al., Nucleic Acid Res. 15:9627-9639, 1987.

An expression cassette including a disclosed a pest resistance gene, such a nucleic acid sequence encoding GmSAMT1 (such as set forth in SEQ ID NO: 1 or a degenerate variant thereof) and/or GmSABP2-1 such as set forth in SEQ ID NO: 2 or a degenerate variant thereof) or functional fragment thereof, operably linked to a promoter sequence may also contain at least additional nucleotide sequence for a gene to be cotransformed into the organism. Alternatively, the additional sequence(s) can be provided on another expression cassette.

Where appropriate, the nucleotide sequences whose expression is desired may be optimized for increased expression in the transformed plant. That is, these nucleotide sequences can be synthesized using plant preferred codons for improved expression. Methods are available in the art for synthesizing plant-preferred nucleotide sequences. See, for example, U.S. Pat. Nos. 5,380,831 and 5,436,391, and Murray et al., Nucleic Acids Res. 17:477-498, 1989.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al., Proc. Nat. Acad. Sci. USA 86:6126-6130, 1989); potyvirus leaders, for example, TEV leader (Tobacco Etch Virus); MDMV leader (Maize Dwarf Mosaic Virus); human immunoglobulin heavy-chain binding protein (BiP) (Macejak and Sarnow Nature 353:90-94, 1991); untranslated leader from the coat protein mRNA of alfalfa mosaic virus (AMV RNA 4) (Jobling and Gehrke Nature 325:622-625, 1987); tobacco mosaic virus leader (TMV) (Gallie et al. Molecular Biology of RNA, pages 237-256, 1989; and maize chlorotic mottle virus leader (MCMV) (Lommel et al., Virology 81:382-385, 1991). See also Della-Cioppa et al., Plant Physiology 84:965-968, 1987. Other methods known to enhance translation and/or mRNA stability can also be utilized, for example, introns, and the like.

In those instances where it is desirable to have the expressed product of the a pest resistance gene directed to a particular organelle, such as the chloroplast or mitochondrion, or secreted at the cell's surface or extracellularly, the expression cassette may further comprise a coding sequence for a transit peptide. Such transit peptides are well known in the art and include, but are not limited to, the transit peptide for the acyl carrier protein, the small subunit of RUBISCO, plant EPSP synthase, and the like.

In preparing the expression cassette, the various DNA fragments may be manipulated by methods known in the art, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, for example, transitions and transversions, may be involved.

The expression cassettes may include reporter genes or selectable marker genes. Examples of suitable reporter genes known in the art can be found in, for example, Jefferson et al. in Plant Molecular Biology Manual, ed. Gelvin et al. (Kluwer Academic Publishers), pp. 1-33, 1991; DeWet et al., Mol. Cell. Biol. 7:725-737, 1987; Goff et al., EMBO J. 9:2517-2522, 1990; and Kain et al., BioTechniques 19:650-655, 1995; and Chiu et al., Current Biology 6:325-330, 1996. Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella et al., EMBO J. 2:987-992, 1983); methotrexate (Herrera Estrella et al., Nature 303:209-213, 1983; Meijer et al., Plant Mol. Biol. 16:807-820, 1991); hygromycin (Waldron et al., Plant Mol. Biol. 5:103-108, 1985; Zhijian et al., Plant Science 108:219-227, 1995); streptomycin (Jones et al., Mol. Gen. Genet. 210:86-91, 1987); spectinomycin (Bretagne-Sagnard et al., Transgenic Res. 5:131-137, 1996); bleomycin (Hille et al., Plant Mol. Biol. 7:171-176, 1990); sulfonamide (Guerineau et al., Plant Mol. Biol. 15:127-136, 1990); bromoxynil (Stalker et al., Science 242:419-423, 1988); glyphosate (Shaw et al., Science 233:478-481, 1986); and phosphinothricin (DeBlock et al., EMBO J. 6:2513-2518, 1987).

Other genes that could serve utility in the recovery of transgenic events but might not be required in the final product would include, but are not limited to, such examples as GUS (b-glucoronidase; Jefferson Plant Mol. Biol. Rep. 5:387, 1987), GFP and other related fluorescent proteins, and luciferase. In some embodiments, an expression cassette is pTh-OFP-35S::GmSAMT1 or pTh-OFP-35S::GmSABP2-1. The following is the sequence of binary vector pTh-OFP-35S::GmSAMT1 showing reverse complemented sequence of GmSAMT1 in bold.

(SEQ ID NO: 3)
TCATGGCTCTTTCAAAGCAAAGTGGGGTCAAAGATGTAATGAACACCGAGCTTCAT
ATGGACGGGATCGTCAATGGACACCCCTTTGAGATAAAAGGAAAAGGAAAGGGAAA
CCCGTACAAGGGTGTGCAGACCATGAAACTTACAGTCATTAAGGGTGCGCCTTTGC
CATTTTCTATTGACATTTTGCTGCCTCAACACATGTATGGAAGCAAGCCATTTATT
AAGTATCCTGAGAGTATCCCAGACTACATCAAGTTGTCATTTCCCGAGGGAATCAC
ATGGGAAAGGTCCATGACCTTTGAAGATGGTGCAGTGTGCACTGTCTCTAACGACT
CCAGGCTCGATGGCGACTCTTTCATCTACGAAGTCAGGTTTCTTGGCGTGAACTTT
CCCCGAGATGGACCTGTTATGCAGAAGAAGACGCGAGGCTGGGACCCGTCCACAGA
GAGACTGTATGAGTGTGGTGGGTGGCAGAGAGGAGACGTCCACATGGCCTTGAAGT
TGGAGAACGGTGGCCATTATACGTGCGACTTCAAAACTACTTACAAATCAAAGAAG
GGCTTGAAGGTGCCACCGTATCACTTCGTTGACCACAAACTAGATTTACTGAGCCA
CAACACCGATGGTGCTACCTTTGAAGAGTTTGAACAACGAGAAATTGCACATGCAC
ATCTTTCTAACTTACCGGTAGCCCATCACCATCACCATCACTAAGATCGTTCAAAC
ATTTGGCAATAAAGTTTCTTAAGATTGAATCCTGTTGCCGGTCTTGCGATGATTAT
CATATAATTTCTGTTGAATTACGTTAAGCATGTAATAATTAACATGTAATGCATGA
CGTTATTTATGAGATGGGTTTTTATGATTAGAGTCCCGCAATTATACATTTAATAC
GCGATAGAAAACAAAATATAGCGCGCAAACTAGGATAAATTATCGCGCGCGGTGTC
ATCTATGTTACTAGATCCCATGGCTACTACTAAGCATTTGGCTCTTGCCATCCTTG
TCCTCCTTAGCATTGGTATGACCACCAGTGCAAGAACCCTCCTAGATCTGAGGGTA
AATTTCTAGTTTTTCTCCTTCATTTTCTTGGTTAGGACCCTTTTCTCTTTTTATTT
TTTTGAGCTTTGATCTTTCTTTAAACTGATCTATTTTTTAATTGATTGGTTATGGT
GTAAATATTACATAGCTTTAACTGATAATCTGATTACTTTATTTCGTGTGTCTATG
ATGATGATGATAGTTACAGAACCGACGAACTAGTCTGTACCCGATCAACACCGAGA
CCCGTGGCGTCTTCGACCTCAATGGCGTCTGGAACTTCAAGCTGGACTACGGGAAA
GGACTGGAAGAGAAGTGGTACGAAAGCAAGCTGACCGACACTATTAGTATGGCCGT
CCCAAGCAGTTACAATGACATTGGCGTGACCAAGGAAATCCGCAACCATATCGGAT
ATGTCTGGTACGAACGTGAGTTCACGGTGCCGGCCTATCTGAAGGATCAGCGTATC
GTGCTCCGCTTCGGCTCTGCAACTCACAAAGCAATTGTCTATGTCAATGGTGAGCT
GGTCGTGGAGCACAAGGGCGGATTCCTGCCATTCGAAGCGGAAATCAACAACTCGC
TGCGTGATGGCATGAATCGCGTCACCGTCGCCGTGGACAACATCCTCGACGATAGC
ACCCTCCCGGTGGGGCTGTACAGCGAGCGCCACGAAGAGGGCCTCGGAAAAGTCAT
TCGTAACAAGCCGAACTTCGACTTCTTCAACTATGCAGGCCTGCACCGTCCGGTGA
AAATCTACACGACCCCGTTTACGTACGTCGAGGACATCTCGGTTGTGACCGACTTC
AATGGCCCAACCGGGACTGTGACCTATACGGTGGACTTTCAAGGCAAAGCCGAGAC
CGTGAAAGTGTCGGTCGTGGATGAGGAAGGCAAAGTGGTCGCAAGCACCGAGGGCC
TGAGCGGTAACGTGGAGATTCCGAATGTCATCCTCTGGGAACCACTGAACACGTAT
CTCTACCAGATCAAAGTGGAACTGGTGAACGACGGACTGACCATCGATGTCTATGA
AGAGCCGTTCGGCGTGCGGACCGTGGAAGTCAACGACGGCAAGTTCCTCATCAACA
ACAAACCGTTCTACTTCAAGGGCTTTGGCAAACATGAGGACACTCCTATCAACGGC
CGTGGCTTTAACGAAGCGAGCAATGTGATGGATTTCAATATCCTCAAATGGATCGG
CGCCAACAGCTTCCGGACCGCACACTATCCGTACTCTGAAGAGTTGATGCGTCTTG
CGGATCGCGAGGGTCTGGTCGTGATCGACGAGACTCCGGCAGTTGGCGTGCACCTC
AACTTCATGGCCACCACGGGACTCGGCGAAGGCAGCGAGCGCGTCAGTACCTGGGA
GAAGATTCGGACGTTTGAGCACCATCAAGACGTTCTCCGTGAACTGGTGTCTCGTG
ACAAGAACCATCCAAGCGTCGTGATGTGGAGCATCGCCAACGAGGCGGCGACTGAG
GAAGAGGGCGCGTACGAGTACTTCAAGCCGTTGGTGGAGCTGACCAAGGAACTCGA
CCCACAGAAGCGTCCGGTCACGATCGTGCTGTTTGTGATGGCTACCCCGGAGACGG
ACAAAGTCGCCGAACTGATTGACGTCATCGCGCTCAATCGCTATAACGGATGGTAC
TTCGATGGCGGTGATCTCGAAGCGGCCAAAGTCCATCTCCGCCAGGAATTTCACGC
GTGGAACAAGCGTTGCCCAGGAAAGCCGATCATGATCACTGAGTACGGCGCAGACA
CCGTTGCGGGCTTTCACGACATTGATCCAGTGATGTTCACCGAGGAATATCAAGTC
GAGTACTACCAGGCGAACCACGTCGTGTTCGATGAGTTTGAGAACTTCGTGGGTGA
GCAAGCGTGGAACTTCGCGGACTTCGCGACCTCTCAGGGCGTGATGCGCGTCCAAG
GAAACAAGAAGGGCGTGTTCACTCGTGACCGCAAGCCGAAGCTCGCCGCGCACGTC
TTTCGCGAGCGCTGGACCAACATTCCAGATTTCGGCTACAAGAACGCTAGCCATCA
CCATCACCATCACGTGTGAATTGGTGACCAGCTCGAATTTCCCCGATCGTTCAAAC
ATTTGGCAATAAAGTTTCTTAAGATTGAATCCTGTTGCCGGTCTTGCGATGATTAT
CATATAATTTCTGTTGAATTACGTTAAGCATGTAATAATTAACATGTAATGCATGA
CGTTATTTATGAGATGGGTTTTTATGATTAGAGTCCCGCAATTATACATTTAATAC
GCGATAGAAAACAAAATATAGCGCGCAAACTAGGATAAATTATCGCGCGCGGTGTC
ATCTATGTTACTAGATCGGGAATTAAACTATCAGTGTTTGACAGGATATATTGGCG
GGTAAACCTAAGAGAAAAGAGCGTTTATTAGAATAACGGATATTTAAAAGGGCGTG
AAAAGGTTTATCCGTTCGTCCATTTGTATGTGCATGCCAACCACAGGGTTCCCCTC
GGGATCAAAGTACTTTGATCCAACCCCTCCGCTGCTATAGTGCAGTCGGCTTCTGA
CGTTCAGTGCAGCCGTCTTCTGAAAACGACATGTCGCACAAGTCCTAAGTTACGCG
ACAGGCTGCCGCCCTGCCCTTTTCCTGGCGTTTTCTTGTCGCGTGTTTTAGTCGCA
TAAAGTAGAATACTTGCGACTAGAACCGGAGACATTACGCCATGAACAAGAGCGCC
GCCGCTGGCCTGCTGGGCTATGCCCGCGTCAGCACCGACGACCAGGACTTGACCAA
CCAACGGGCCGAACTGCACGCGGCCGGCTGCACCAAGCTGTTTTCCGAGAAGATCA
CCGGCACCAGGCGCGACCGCCCGGAGCTGGCCAGGATGCTTGACCACCTACGCCCT
GGCGACGTTGTGACAGTGACCAGGCTAGACCGCCTGGCCCGCAGCACCCGCGACCT
ACTGGACATTGCCGAGCGCATCCAGGAGGCCGGCGCGGGCCTGCGTAGCCTGGCAG
AGCCGTGGGCCGACACCACCACGCCGGCCGGCCGCATGGTGTTGACCGTGTTCGCC
GGCATTGCCGAGTTCGAGCGTTCCCTAATCATCGACCGCACCCGGAGCGGGCGCGA
GGCCGCCAAGGCCCGAGGCGTGAAGTTTGGCCCCCGCCCTACCCTCACCCCGGCAC
AGATCGCGCACGCCCGCGAGCTGATCGACCAGGAAGGCCGCACCGTGAAAGAGGCG
GCTGCACTGCTTGGCGTGCATCGCTCGACCCTGTACCGCGCACTTGAGCGCAGCGA
GGAAGTGACGCCCACCGAGGCCAGGCGGCGCGGTGCCTTCCGTGAGGACGCATTGA
CCGAGGCCGACGCCCTGGCGGCCGCCGAGAATGAACGCCAAGAGGAACAAGCATGA
AACCGCACCAGGACGGCCAGGACGAACCGTTTTTCATTACCGAAGAGATCGAGGCG
GAGATGATCGCGGCCGGGTACGTGTTCGAGCCGCCCGCGCACGTCTCAACCGTGCG
GCTGCATGAAATCCTGGCCGGTTTGTCTGATGCCAAGCTGGCGGCCTGGCCGGCCA
GCTTGGCCGCTGAAGAAACCGAGCGCCGCCGTCTAAAAAGGTGATGTGTATTTGAG
TAAAACAGCTTGCGTCATGCGGTCGCTGCGTATATGATGCGATGAGTAAATAAACA
AATACGCAAGGGGAACGCATGAAGGTTATCGCTGTACTTAACCAGAAAGGCGGGTC
AGGCAAGACGACCATCGCAACCCATCTAGCCCGCGCCCTGCAACTCGCCGGGGCCG
ATGTTCTGTTAGTCGATTCCGATCCCCAGGGCAGTGCCCGCGATTGGGCGGCCGTG
CGGGAAGATCAACCGCTAACCGTTGTCGGCATCGACCGCCCGACGATTGACCGCGA
CGTGAAGGCCATCGGCCGGCGCGACTTCGTAGTGATCGACGGAGCGCCCCAGGCGG
CGGACTTGGCTGTGTCCGCGATCAAGGCAGCCGACTTCGTGCTGATTCCGGTGCAG
CCAAGCCCTTACGACATATGGGCCACCGCCGACCTGGTGGAGCTGGTTAAGCAGCG
CATTGAGGTCACGGATGGAAGGCTACAAGCGGCCTTTGTCGTGTCGCGGGCGATCA
AAGGCACGCGCATCGGCGGTGAGGTTGCCGAGGCGCTGGCCGGGTACGAGCTGCCC
ATTCTTGAGTCCCGTATCACGCAGCGCGTGAGCTACCCAGGCACTGCCGCCGCCGG
CACAACCGTTCTTGAATCAGAACCCGAGGGCGACGCTGCCCGCGAGGTCCAGGCGC
TGGCCGCTGAAATTAAATCAAAACTCATTTGAGTTAATGAGGTAAAGAGAAAATGA
GCAAAAGCACAAACACGCTAAGTGCCGGCCGTCCGAGCGCACGCAGCAGCAAGGCT
GCAACGTTGGCCAGCCTGGCAGACACGCCAGCCATGAAGCGGGTCAACTTTCAGTT
GCCGGCGGAGGATCACACCAAGCTGAAGATGTACGCGGTACGCCAAGGCAAGACCA
TTACCGAGCTGCTATCTGAATACATCGCGCAGCTACCAGAGTAAATGAGCAAATGA
ATAAATGAGTAGATGAATTTTAGCGGCTAAAGGAGGCGGCATGGAAAATCAAGAAC
AACCAGGCACCGACGCCGTGGAATGCCCCATGTGTGGAGGAACGGGCGGTTGGCCA
GGCGTAAGCGGCTGGGTTGTCTGCCGGCCCTGCAATGGCACTGGAACCCCCAAGCC
CGAGGAATCGGCGTGACGGTCGCAAACCATCCGGCCCGGTACAAATCGGCGCGGCG
CTGGGTGATGACCTGGTGGAGAAGTTGAAGGCCGCGCAGGCCGCCCAGCGGCAACG
CATCGAGGCAGAAGCACGCCCCGGTGAATCGTGGCAAGCGGCCGCTGATCGAATCC
GCAAAGAATCCCGGCAACCGCCGGCAGCCGGTGCGCCGTCGATTAGGAAGCCGCCC
AAGGGCGACGAGCAACCAGATTTTTTCGTTCCGATGCTCTATGACGTGGGCACCCG
CGATAGTCGCAGCATCATGGACGTGGCCGTTTTCCGTCTGTCGAAGCGTGACCGAC
GAGCTGGCGAGGTGATCCGCTACGAGCTTCCAGACGGGCACGTAGAGGTTTCCGCA
GGGCCGGCCGGCATGGCCAGTGTGTGGGATTACGACCTGGTACTGATGGCGGTTTC
CCATCTAACCGAATCCATGAACCGATACCGGGAAGGGAAGGGAGACAAGCCCGGCC
GCGTGTTCCGTCCACACGTTGCGGACGTACTCAAGTTCTGCCGGCGAGCCGATGGC
GGAAAGCAGAAAGACGACCTGGTAGAAACCTGCATTCGGTTAAACACCACGCACGT
TGCCATGCAGCGTACGAAGAAGGCCAAGAACGGCCGCCTGGTGACGGTATCCGAGG
GTGAAGCCTTGATTAGCCGCTACAAGATCGTAAAGAGCGAAACCGGGCGGCCGGAG
TACATCGAGATCGAGCTAGCTGATTGGATGTACCGCGAGATCACAGAAGGCAAGAA
CCCGGACGTGCTGACGGTTCACCCCGATTACTTTTTGATCGATCCCGGCATCGGCC
GTTTTCTCTACCGCCTGGCACGCCGCGCCGCAGGCAAGGCAGAAGCCAGATGGTTG
TTCAAGACGATCTACGAACGCAGTGGCAGCGCCGGAGAGTTCAAGAAGTTCTGTTT
CACCGTGCGCAAGCTGATCGGGTCAAATGACCTGCCGGAGTACGATTTGAAGGAGG
AGGCGGGGCAGGCTGGCCCGATCCTAGTCATGCGCTACCGCAACCTGATCGAGGGC
GAAGCATCCGCCGGTTCCTAATGTACGGAGCAGATGCTAGGGCAAATTGCCCTAGC
AGGGGAAAAAGGTCGAAAAGGTCTCTTTCCTGTGGATAGCACGTACATTGGGAACC
CAAAGCCGTACATTGGGAACCGGAACCCGTACATTGGGAACCCAAAGCCGTACATT
GGGAACCGGTCACACATGTAAGTGACTGATATAAAAGAGAAAAAAGGCGATTTTTC
CGCCTAAAACTCTTTAAAACTTATTAAAACTCTTAAAACCCGCCTGGCCTGTGCAT
AACTGTCTGGCCAGCGCACAGCCGAAGAGCTGCAAAAAGCGCCTACCCTTCGGTCG
CTGCGCTCCCTACGCCCCGCCGCTTCGCGTCGGCCTATCGCGGCCGCTGGCCGCTC
AAAAATGGCTGGCCTACGGCCAGGCAATCTACCAGGGCGCGGACAAGCCGCGCCGT
CGCCACTCGACCGCCGGCGCCCACATCAAGGCACCCTGCCTCGCGCGTTTCGGTGA
TGACGGTGAAAACCTCTGACACATGCAGCTCCCGGAGACGGTCACAGCTTGTCTGT
AAGCGGATGCCGGGAGCAGACAAGCCCGTCAGGGCGCGTCAGCGGGTGTTGGCGGG
TGTCGGGGCGCAGCCATGACCCAGTCACGTAGCGATAGCGGAGTGTATACTGGCTT
AACTATGCGGCATCAGAGCAGATTGTACTGAGAGTGCACCATATGCGGTGTGAAAT
ACCGCACAGATGCGTAAGGAGAAAATACCGCATCAGGCGCTCTTCCGCTTCCTCGC
TCACTGACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAACATGTG
AGCAAAAGGCCAGCAAAAGGCCAGGAACCGTAAAAAGGCCGCGTTGCTGGCGTTTT
TCCATAGGCTCCGCCCCCCTGACGAGCATCACAAAAATCGACGCTCAAGTCAGAGG
TGGCGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCGGT
GCTACAGAGTTCTTGAAGTGGTGGCCTAACTACGGCTACACTAGAAGGACAGTATT
TGGTATCTGCGCTCTGCTGAAGCCAGTTACCTTCGGAAAAAGAGTTGGTAGCTCTT
GATCCGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGGGGTC
TGACGCTCAGTGGAACGAAAACTCACGTTAAGGGATTTTGGTCATGCATTCTAGGT
ACTAAAACAATTCATCCAGTAAAATATAATATTTTATTTTCTCCCAATCAGGCTTG
ATCCCCAGTAAGTCAAAAAATAGCTCGACATACTGTTCTTCCCCGATATCCTCCCT
GATCGACCGGACGCAGAAGGCAATGTCATACCACTTGTCCGCCCTGCCGCTTCTCC
CAAGATCAATAAAGCCACTTACTTTGCCATCTTTCACAAAGATGTTGCTGTCTCCC
AGGTCGCCGTGGGAAAAGACAAGTTCCTCTTCGGGCTTTTCCGTCTTTAAAAAATC
ATACAGCTCGCGCGGATCTTTAAATGGAGTGTCTTCTTCCCAGTTTTCGCAATCCA
CATCGGCCAGATCGTTATTCAGTAAGTAATCCAATTCGGCTAAGCGGCTGTCTAAG
CTATTCGTATAGGGACAATCCGATATGTCGATGGAGTGAAAGAGCCTGATGCACTC
CGCATACAGCTCGATAATCTTTTCAGGGCTTTGTTCATCTTCATACTCTTCCGAGC
AAAGGACGCCATCGGCCTCACTCATGAGCAGATTGCTCCAGCCATCATGCCGTTCA
AAGTGCAGGACCTTTGGAACAGGCAGCTTTCCTTCCAGCCATAGCATCATGTCCTT
TTCCCGTTCCACATCATAGGTGGTCCCTTTATACCGGCTGTCCGTCATTTTTAAAT
ATAGGTTTTCATTTTCTCCCACCAGCTTATATACCTTAGCAGGAGACATTCCTTCC
GTATCTTTTACGCAGCGGTATTTTTCGATCAGTTTTTTCAATTCCGGTGATATTCT
CATTTTAGCCATTTATTATTTCCTTCCTCTTTTCTACAGTATTTAAAGATACCCCA
AGAAGCTAATTATAACAAGACGAACTCCAATTCACTGTTCCTTGCATTCTAAAACC
TTAAATACCAGAAAACAGCTTTTTCAAAGTTGTTTTCAAAGTTGGCGTATAACATA
GTATCGACGGAGCCGATTTTGAAACCGCGGTGATCACAGGCAGCAACGCTCTGTCA
TCGTTACAATCAACATGCTACCCTCCGCGAGATCATCCGTGTTTCAAACCCGGCAG
CTTAGTTGCCGTTCTTCCGAATAGCATCGGTAACATGAGCAAAGTCTGCCGCCTTA
CAACGGCTCTCCCGCTGACGCCGTCCCGGACTGATGGGCTGCCTGTATCGAGTGGT
GATTTTGTGCCGAGCTGCCGGTCGGGGAGCTGTTGGCTGGCTGGTGGCAGGATATA
TTGTGGTGTAAACAAATTGACGCTTAGACAACTTAATAACACATTGCGGACGTTTT
TAATGTACTGAATTAACGCCGAATTAATTCGGGGGATCTGGATTTTAGTACTGGAT
TTTGGTTTTAGGAATTAGAAATTTTATTGATAGAAGTATTTTACAAATACAAATAC
ATACTAAGGGTTTCTTATATGCTCAACACATGAGCGAAACCCTATAGGAACCCTAA
TTCCCTTATCTGGGAACTACTCACACATTATTATGGAGAAACTCGAGCTTGTCGAT
CGACAGATCCGGTCGGCATCTACTCTATTTCTTTGCCCTCGGACGAGTGCTGGGGC
GTCGGTTTCCACTATCGGCGAGTACTTCTACACAGCCATCGGTCCAGACGGCCGCG
CTTCTGCGGGCGATTTGTGTACGCCCGACAGTCCCGGCTCCGGATCGGACGATTGC
GTCGCATCGACCCTGCGCCCAAGCTGCATCATCGAAATTGCCGTCAACCAAGCTCT
GATAGAGTTGGTCAAGACCAATGCGGAGCATATACGCCCGGAGTCGTGGCGATCCT
GCAAGCTCCGGATGCCTCCGCTCGAAGTAGCGCGTCTGCTGCTCCATACAAGCCAA
CCACGGCCTCCAGAAGAAGATGTTGGCGACCTCGTATTGGGAATCCCCGAACATCG
CCTCGCTCCAGTCAATGACCGCTGTTATGCGGCCATTGTCCGTCAGGACATTGTTG
GAGCCGAAATCCGCGTGCACGAGGTGCCGGACTTCGGGGCAGTCCTCGGCCCAAAG
CATCAGCTCATCGAGAGCCTGCGCGACGGACGCACTGACGGTGTCGTCCATCACAG
TTTGCCAGTGATACACATGGGGATCAGCAATCGCGCATATGAAATCACGCCATGTA
GTGTATTGACCGATTCCTTGCGGTCCGAATGGGCCGAACCCGCTCGTCTGGCTAAG
ATCGGCCGCAGCGATCGCATCCATAGCCTCCGCGACCGGTTGTAGAACAGCGGGCA
GTTCGGTTTCAGGCAGGTCTTGCAACGTGACACCCTGTGCACGGCGGGAGATGCAA
TAGGTCAGGCTCTCGCTAAACTCCCCAATGTCAAGCACTTCCGGAATCGGGAGCGC
GGCCGATGCAAAGTGCCGATAAACATAACGATCTTTGTAGAAACCATCGGCGCAGC
TATTTACCCGCAGGACATATCCACGCCCTCCTACATCGAAGCTGAAAGCACGAGAT
TCTTCGCCCTCCGAGAGCTGCATCAGGTCGGAGACGCTGTCGAACTTTTCGATCAG
AAACTTCTCGACAGACGTCGCGGTGAGTTCAGGCTTTTTCATATCTCATTGCCCCC
CGGGATCTGCGAAAGCTCGAGAGAGATAGATTTGTAGAGAGAGACTGGTGATTTCA
GCGTGTCCTCTCCAAATGAAATGAACTTCCTTATATAGAGGAAGGTCTTGCGAAGG
ATAGTGGGATTGTGCGTCATCCCTTACGTCAGTGGAGATATCACATCAATCCACTT
GCTTTGAAGACGTGGTTGGAACGTCTTCTTTTTCCACGATGCTCCTCGTGGGTGGG
GGTCCATCTTTGGGACCACTGTCGGCAGAGGCATCTTGAACGATAGCCTTTCCTTT
ATCGCAATGATGGCATTTGTAGGTGCCACCTTCCTTTTCTACTGTCCTTTTGATGA
AGTGACAGATAGCTGGGCAATGGAATCCGAGGAGGTTTCCCGATATTACCCTTTGT
TGAAAAGTCTCAATAGCCCTTTGGTCTTCTGAGACTGTATCTTTGATATTCTTGGA
GTAGACGAGAGTGTCGTGCTCCACCATGTTATCACATCAATCCACTTGCTTTGAAG
ACGTGGTTGGAACGTCTTCTTTTTCCACGATGCTCCTCGTGGGTGGGGGTCCATCT
TTGGGACCACTGTCGGCAGAGGCATCTTGAACGATAGCCTTTCCTTTATCGCAATG
ATGGCATTTGTAGGTGCCACCTTCCTTTTCTACTGTCCTTTTGATGAAGTGACAGA
TAGCTGGGCAATGGAATCCGAGGAGGTTTCCCGATATTACCCTTTGTTGAAAAGTC
TCAATAGCCCTTTGGTCTTCTGAGACTGTATCTTTGATATTCTTGGAGTAGACGAG
AGTGTCGTGCTCCACCATGTTGGCAAGCTGCTCTAGCCAATACGCAAACCGCCTCT
CCCCGCGCGTTGGCCGATTCATTAATGCAGCTGGCACGACAGGTTTCCCGACTGGA
AAGCGGGCAGTGAGCGCAACGCAATTAATGTGAGTTAGCTCACTCATTAGGCACCC
CAGGCTTTACACTTTATGCTTCCGGCTCGTATGTTGTGTGGAATTGTGAGCGGATA
ACAATTTCACACAGGAAACAGCTATGACCATGATTACGAATTCCCGATCTAGTAAC
ATAGATGACACCGCGCGCGATAATTTATCCTAGTTTGCGCGCTATATTTTGTTTTC
TATCGCGTATTAAATGTATAATTGCGGGACTCTAATCATAAAAACCCATCTCATAA
ATAACGTCATGCATTACATGTTAATTATTACATGCTTAACGTAATTCAACAGAAAT
TATATGATAATCATCGCAAGACCGGCAACAGGATTCAATCTTAAGAAACTTTATTG
CCAAATGTTTGAACGATCGGGGAAATTCGAGCTCTTATGCTTTTCTAGTCAATAAT
ATGGTAACATTGATGAACTTGGTTTTCTCCTTGGACATACGTTCAGCCAAGATTTG
CTGGTAGCGGCAAAAAACCTCTTCAATGATAGCTTCACCAAAGTGGCTAATCAGCA
TAGGTTCTGCCACAGCCCTCATGCACTGTGCCACATTGTATCCACCATCACTAAGT
GATTCAGACCTTTCAGATTCAAAGTCTAGAGCATTCCACTCATCGAGAGCATTCCA
ATTCACCTCAGAAACCTCTAGACGATTGATGGCGAATGATCCTTCTTTAAGAACTT
CCAATTTCACTTCAGATGGGGATGGAGTGTATTGAGGGATGTTAAAAGTATCTAAT
TGCTCTTCTCTTATGATTCCCTGCAAGACCATATCACTAAGAGCAGTAGCCAAAAG
CTCCCAAATGTAGCAACCATCCTTGCTAGATGGATCATCGCTTCTTCTTCCCAAAA
ATGTGAGAACCATGCGTCCTCCTTCAACCAATTCCTCTGCACGACACTTGAGAAAA
AGAGAGAAATCTCTCTGAAATTGCTCATAGTAAGCTCTCACAACATTTTTGGGGCT
CGTACTGCCTATGTAAACATTGCCCTTGTTGTTGTCTACACCCTCAGGAACCTTAG
ATAGCCATTGAAGGCTGTATGAGGAATGGACAAAATGAAGACTTTGATATGGGAAA
ACCCTTCCATAGAAAGAACCGGGAACACCCGAGAAGTAGCATGGACCGATCCCACT
TTCCATTTCATCACACAATTTCTCTTTGAAGCTGTCAAGAGACTTGAAGATGTTGT
TGAAGTCATTCCCAGGGAGATCATTCAGAAAGACTTTGTATTCTGGAGATTTGTGG
TTCAGCTCTCGGCAAAGCTTCTCCACAATTTTTATGAATTCAGACACAACAAATAA
AGTGTTTGGTCCAGAAGAGCAACCCAAATCTGCAATTGCCAAGCTTCTGGGGTGTG
TGCTGCAATAGAGGCTTCTTATGGCTTCCTCTCTTATGGGCTTTGTCAAACAAATC
ACCTTTTGCTGAACAAGGGAGTTGTTTGCATAGCTTGCGTCTCCAACGCCACCGTT
CATGTGGAGTACCTGTGCTACTTCCATGGATCCTCTAGAGTCCCCCGTGTTCTCTC
CAAATGAAATGAACTTCCTTATATAGAGGAAGGGTCTTGCGAAGGATAGTGGGATT
GTGCGTCATCCCTTACGTCAGTGGAGATATCACATCAATCCACTTGCTTTGAAGAC
GTGGTTGGAACGTCTTCTTTTTCCACGATGCTCCTCGTGGGTGGGGGTCCATCTTT
GGGACCACTGTCGGCAGAGGCATCTTCAACGATGGCCTTTCCTTTATCGCAATGAT
GGCATTTGTAGGAGCCACCTTCCTTTTCCACTATCTTCACAATAAAGTGACAGATA
GCTGGGCAATGGAATCCGAGGAGGTTTCCGGATATTACCCTTTGTTGAAAAGTCTC
AATTGCCCTTTGGTCTTCTGAGACTGTATCTTTGATATTTTTGGAGTAGACAAGTG
TGTCGTGCTCCACCATGTTGACGAAGATTTTCTTCTTGTCATTGAGTCGTAAGAGA
CTCTGTATGAACTGTTCGCCAGTCTTTACGGCGAGTTCTGTTAGGTCCTCTATTTG
AATCTTTGACTCCATGGCCTTTGATTCAGTGGGAACTACCTTTTTAGAGACTCCAA
TCTCTATTACTTGCCTTGGTTTGTGAAGCAAGCCTTGAATCGTCCATACTGGAATA
GTACTTCTGATCTTGAGAAATATATCTTTCTCTGTGTTCTTGATGCAGTTAGTCCT
GAATCTTTTGACTGCATCTTTAACCTTCTTGGGAAGGTATTTGATTTCCTGGAGAT
TATTGCTCGGGTAGATCGTCTTGATGAGACCTGCTGCGTAAGCCTCTCTAACCATC
TGTGGGTTAGCATTCTTTCTGAAATTGAAAAGGCTAATCTGGGGACCTGCAGGCAT
GCAAGCTTGGCACTGGCCGTCGTTTTACAACGTCGTGACTGGGAAAACCCTGGCGT
TACCCAACTTAATCGCCTTGCAGCACATCCCCCTTTCGCCAGCTGGCGTAATAGCG
AAGAGGCCCGCACCGATCGCCCTTCCCAACAGTTGCGCAGCCTGAATGGCGAATGC
TAGAGCAGCTTGAGCTTGGATCAGATTGTCGTTTCCCGCCTTCAGTTTAGCTTCAT
GGAGTCAAAGATTCAAATAGAGGACCTAACAGAACTCGCCGTAAAGACTGGCGAAC
AGTTCATACAGAGTCTCTTACGACTCAATGACAAGAAGAAAATCTTCGTCAACATG
GTGGAGCACGACACACTTGTCTACTCCAAAAATATCAAAGATACAGTCTCAGAAGA
CCAAAGGGCAATTGAGACTTTTCAACAAAGGGTAATATCCGGAAACCTCCTCGGAT
TCCATTGCCCAGCTATCTGTCACTTTATTGTGAAGATAGTGGAAAAGGAAGGTGGC
TCCTACAAATGCCATCATTGCGATAAAGGAAAGGCCATCGTTGAAGATGCCTCTGC
CGACAGTGGTCCCAAAGATGGACCCCCACCCACGAGGAGCATCGTGGAAAAAGAAG
ACGTTCCAACCACGTCTTCAAAGCAAGTGGATTGATGTGATATCTCCACTGACGTA
AGGGATGACGCACAATCCCACTATCCTTCGCAAGACCCTTCCTCTATATAAGGAAG
TTCATTTCATTTGGAGAGAACACGGGGGACTCTTGAC

The following is the sequence of binary vector pJL-OFP-35S::GmSABP2-1, showing reverse complemented sequence of GmSABP2-1 in bold.

(SEQ ID NO: 4)
TCATGGCTCTTTCAAAGCAAAGTGGGGTCAAAGATGTAATGAACACCGAGCTTCAT
ATGGACGGGATCGTCAATGGACACCCCTTTGAGATAAAAGGAAAAGGAAAGGGAAA
CCCGTACAAGGGTGTGCAGACCATGAAACTTACAGTCATTAAGGGTGCGCCTTTGC
CATTTTCTATTGACATTTTGCTGCCTCAACACATGTATGGAAGCAAGCCATTTATT
AAGTATCCTGAGAGTATCCCAGACTACATCAAGTTGTCATTTCCCGAGGGAATCAC
ATGGGAAAGGTCCATGACCTTTGAAGATGGTGCAGTGTGCACTGTCTCTAACGACT
CCAGGCTCGATGGCGACTCTTTCATCTACGAAGTCAGGTTTCTTGGCGTGAACTTT
CCCCGAGATGGACCTGTTATGCAGAAGAAGACGCGAGGCTGGGACCCGTCCACAGA
GAGACTGTATGAGTGTGGTGGGTGGCAGAGAGGAGACGTCCACATGGCCTTGAAGT
TGGAGAACGGTGGCCATTATACGTGCGACTTCAAAACTACTTACAAATCAAAGAAG
GGCTTGAAGGTGCCACCGTATCACTTCGTTGACCACAAACTAGATTTACTGAGCCA
CAACACCGATGGTGCTACCTTTGAAGAGTTTGAACAACGAGAAATTGCACATGCAC
ATCTTTCTAACTTACCGGTAGCCCATCACCATCACCATCACTAAGATCGTTCAAAC
ATTTGGCAATAAAGTTTCTTAAGATTGAATCCTGTTGCCGGTCTTGCGATGATTAT
CATATAATTTCTGTTGAATTACGTTAAGCATGTAATAATTAACATGTAATGCATGA
CGTTATTTATGAGATGGGTTTTTATGATTAGAGTCCCGCAATTATACATTTAATAC
GCGATAGAAAACAAAATATAGCGCGCAAACTAGGATAAATTATCGCGCGCGGTGTC
ATCTATGTTACTAGATCCCATGGCTACTACTAAGCATTTGGCTCTTGCCATCCTTG
TCCTCCTTAGCATTGGTATGACCACCAGTGCAAGAACCCTCCTAGATCTGAGGGTA
AATTTCTAGTTTTTCTCCTTCATTTTCTTGGTTAGGACCCTTTTCTCTTTTTATTT
TTTTGAGCTTTGATCTTTCTTTAAACTGATCTATTTTTTAATTGATTGGTTATGGT
GTAAATATTACATAGCTTTAACTGATAATCTGATTACTTTATTTCGTGTGTCTATG
ATGATGATGATAGTTACAGAACCGACGAACTAGTCTGTACCCGATCAACACCGAGA
CCCGTGGCGTCTTCGACCTCAATGGCGTCTGGAACTTCAAGCTGGACTACGGGAAA
GGACTGGAAGAGAAGTGGTACGAAAGCAAGCTGACCGACACTATTAGTATGGCCGT
CCCAAGCAGTTACAATGACATTGGCGTGACCAAGGAAATCCGCAACCATATCGGAT
ATGTCTGGTACGAACGTGAGTTCACGGTGCCGGCCTATCTGAAGGATCAGCGTATC
GTGCTCCGCTTCGGCTCTGCAACTCACAAAGCAATTGTCTATGTCAATGGTGAGCT
GGTCGTGGAGCACAAGGGCGGATTCCTGCCATTCGAAGCGGAAATCAACAACTCGC
TGCGTGATGGCATGAATCGCGTCACCGTCGCCGTGGACAACATCCTCGACGATAGC
ACCCTCCCGGTGGGGCTGTACAGCGAGCGCCACGAAGAGGGCCTCGGAAAAGTCAT
TCGTAACAAGCCGAACTTCGACTTCTTCAACTATGCAGGCCTGCACCGTCCGGTGA
AAATCTACACGACCCCGTTTACGTACGTCGAGGACATCTCGGTTGTGACCGACTTC
AATGGCCCAACCGGGACTGTGACCTATACGGTGGACTTTCAAGGCAAAGCCGAGAC
CGTGAAAGTGTCGGTCGTGGATGAGGAAGGCAAAGTGGTCGCAAGCACCGAGGGCC
TGAGCGGTAACGTGGAGATTCCGAATGTCATCCTCTGGGAACCACTGAACACGTAT
CTCTACCAGATCAAAGTGGAACTGGTGAACGACGGACTGACCATCGATGTCTATGA
AGAGCCGTTCGGCGTGCGGACCGTGGAAGTCAACGACGGCAAGTTCCTCATCAACA
ACAAACCGTTCTACTTCAAGGGCTTTGGCAAACATGAGGACACTCCTATCAACGGC
CGTGGCTTTAACGAAGCGAGCAATGTGATGGATTTCAATATCCTCAAATGGATCGG
CGCCAACAGCTTCCGGACCGCACACTATCCGTACTCTGAAGAGTTGATGCGTCTTG
CGGATCGCGAGGGTCTGGTCGTGATCGACGAGACTCCGGCAGTTGGCGTGCACCTC
AACTTCATGGCCACCACGGGACTCGGCGAAGGCAGCGAGCGCGTCAGTACCTGGGA
GAAGATTCGGACGTTTGAGCACCATCAAGACGTTCTCCGTGAACTGGTGTCTCGTG
ACAAGAACCATCCAAGCGTCGTGATGTGGAGCATCGCCAACGAGGCGGCGACTGAG
GAAGAGGGCGCGTACGAGTACTTCAAGCCGTTGGTGGAGCTGACCAAGGAACTCGA
CCCACAGAAGCGTCCGGTCACGATCGTGCTGTTTGTGATGGCTACCCCGGAGACGG
ACAAAGTCGCCGAACTGATTGACGTCATCGCGCTCAATCGCTATAACGGATGGTAC
TTCGATGGCGGTGATCTCGAAGCGGCCAAAGTCCATCTCCGCCAGGAATTTCACGC
GTGGAACAAGCGTTGCCCAGGAAAGCCGATCATGATCACTGAGTACGGCGCAGACA
CCGTTGCGGGCTTTCACGACATTGATCCAGTGATGTTCACCGAGGAATATCAAGTC
GAGTACTACCAGGCGAACCACGTCGTGTTCGATGAGTTTGAGAACTTCGTGGGTGA
GCAAGCGTGGAACTTCGCGGACTTCGCGACCTCTCAGGGCGTGATGCGCGTCCAAG
GAAACAAGAAGGGCGTGTTCACTCGTGACCGCAAGCCGAAGCTCGCCGCGCACGTC
TTTCGCGAGCGCTGGACCAACATTCCAGATTTCGGCTACAAGAACGCTAGCCATCA
CCATCACCATCACGTGTGAATTGGTGACCAGCTCGAATTTCCCCGATCGTTCAAAC
ATTTGGCAATAAAGTTTCTTAAGATTGAATCCTGTTGCCGGTCTTGCGATGATTAT
CATATAATTTCTGTTGAATTACGTTAAGCATGTAATAATTAACATGTAATGCATGA
CGTTATTTATGAGATGGGTTTTTATGATTAGAGTCCCGCAATTATACATTTAATAC
GCGATAGAAAACAAAATATAGCGCGCAAACTAGGATAAATTATCGCGCGCGGTGTC
ATCTATGTTACTAGATCGGGAATTAAACTATCAGTGTTTGACAGGATATATTGGCG
GGTAAACCTAAGAGAAAAGAGCGTTTATTAGAATAACGGATATTTAAAAGGGCGTG
AAAAGGTTTATCCGTTCGTCCATTTGTATGTGCATGCCAACCACAGGGTTCCCCTC
GGGATCAAAGTACTTTGATCCAACCCCTCCGCTGCTATAGTGCAGTCGGCTTCTGA
CGTTCAGTGCAGCCGTCTTCTGAAAACGACATGTCGCACAAGTCCTAAGTTACGCG
ACAGGCTGCCGCCCTGCCCTTTTCCTGGCGTTTTCTTGTCGCGTGTTTTAGTCGCA
TAAAGTAGAATACTTGCGACTAGAACCGGAGACATTACGCCATGAACAAGAGCGCC
GCCGCTGGCCTGCTGGGCTATGCCCGCGTCAGCACCGACGACCAGGACTTGACCAA
CCAACGGGCCGAACTGCACGCGGCCGGCTGCACCAAGCTGTTTTCCGAGAAGATCA
CCGGCACCAGGCGCGACCGCCCGGAGCTGGCCAGGATGCTTGACCACCTACGCCCT
GGCGACGTTGTGACAGTGACCAGGCTAGACCGCCTGGCCCGCAGCACCCGCGACCT
ACTGGACATTGCCGAGCGCATCCAGGAGGCCGGCGCGGGCCTGCGTAGCCTGGCAG
AGCCGTGGGCCGACACCACCACGCCGGCCGGCCGCATGGTGTTGACCGTGTTCGCC
GGCATTGCCGAGTTCGAGCGTTCCCTAATCATCGACCGCACCCGGAGCGGGCGCGA
GGCCGCCAAGGCCCGAGGCGTGAAGTTTGGCCCCCGCCCTACCCTCACCCCGGCAC
AGATCGCGCACGCCCGCGAGCTGATCGACCAGGAAGGCCGCACCGTGAAAGAGGCG
GCTGCACTGCTTGGCGTGCATCGCTCGACCCTGTACCGCGCACTTGAGCGCAGCGA
GGAAGTGACGCCCACCGAGGCCAGGCGGCGCGGTGCCTTCCGTGAGGACGCATTGA
CCGAGGCCGACGCCCTGGCGGCCGCCGAGAATGAACGCCAAGAGGAACAAGCATGA
AACCGCACCAGGACGGCCAGGACGAACCGTTTTTCATTACCGAAGAGATCGAGGCG
GAGATGATCGCGGCCGGGTACGTGTTCGAGCCGCCCGCGCACGTCTCAACCGTGCG
GCTGCATGAAATCCTGGCCGGTTTGTCTGATGCCAAGCTGGCGGCCTGGCCGGCCA
GCTTGGCCGCTGAAGAAACCGAGCGCCGCCGTCTAAAAAGGTGATGTGTATTTGAG
TAAAACAGCTTGCGTCATGCGGTCGCTGCGTATATGATGCGATGAGTAAATAAACA
AATACGCAAGGGGAACGCATGAAGGTTATCGCTGTACTTAACCAGAAAGGCGGGTC
AGGCAAGACGACCATCGCAACCCATCTAGCCCGCGCCCTGCAACTCGCCGGGGCCG
ATGTTCTGTTAGTCGATTCCGATCCCCAGGGCAGTGCCCGCGATTGGGCGGCCGTG
CGGGAAGATCAACCGCTAACCGTTGTCGGCATCGACCGCCCGACGATTGACCGCGA
CGTGAAGGCCATCGGCCGGCGCGACTTCGTAGTGATCGACGGAGCGCCCCAGGCGG
CGGACTTGGCTGTGTCCGCGATCAAGGCAGCCGACTTCGTGCTGATTCCGGTGCAG
CCAAGCCCTTACGACATATGGGCCACCGCCGACCTGGTGGAGCTGGTTAAGCAGCG
CATTGAGGTCACGGATGGAAGGCTACAAGCGGCCTTTGTCGTGTCGCGGGCGATCA
AAGGCACGCGCATCGGCGGTGAGGTTGCCGAGGCGCTGGCCGGGTACGAGCTGCCC
ATTCTTGAGTCCCGTATCACGCAGCGCGTGAGCTACCCAGGCACTGCCGCCGCCGG
CACAACCGTTCTTGAATCAGAACCCGAGGGCGACGCTGCCCGCGAGGTCCAGGCGC
TGGCCGCTGAAATTAAATCAAAACTCATTTGAGTTAATGAGGTAAAGAGAAAATGA
GCAAAAGCACAAACACGCTAAGTGCCGGCCGTCCGAGCGCACGCAGCAGCAAGGCT
GCAACGTTGGCCAGCCTGGCAGACACGCCAGCCATGAAGCGGGTCAACTTTCAGTT
GCCGGCGGAGGATCACACCAAGCTGAAGATGTACGCGGTACGCCAAGGCAAGACCA
TTACCGAGCTGCTATCTGAATACATCGCGCAGCTACCAGAGTAAATGAGCAAATGA
ATAAATGAGTAGATGAATTTTAGCGGCTAAAGGAGGCGGCATGGAAAATCAAGAAC
AACCAGGCACCGACGCCGTGGAATGCCCCATGTGTGGAGGAACGGGCGGTTGGCCA
GGCGTAAGCGGCTGGGTTGTCTGCCGGCCCTGCAATGGCACTGGAACCCCCAAGCC
CGAGGAATCGGCGTGACGGTCGCAAACCATCCGGCCCGGTACAAATCGGCGCGGCG
CTGGGTGATGACCTGGTGGAGAAGTTGAAGGCCGCGCAGGCCGCCCAGCGGCAACG
CATCGAGGCAGAAGCACGCCCCGGTGAATCGTGGCAAGCGGCCGCTGATCGAATCC
GCAAAGAATCCCGGCAACCGCCGGCAGCCGGTGCGCCGTCGATTAGGAAGCCGCCC
AAGGGCGACGAGCAACCAGATTTTTTCGTTCCGATGCTCTATGACGTGGGCACCCG
CGATAGTCGCAGCATCATGGACGTGGCCGTTTTCCGTCTGTCGAAGCGTGACCGAC
GAGCTGGCGAGGTGATCCGCTACGAGCTTCCAGACGGGCACGTAGAGGTTTCCGCA
GGGCCGGCCGGCATGGCCAGTGTGTGGGATTACGACCTGGTACTGATGGCGGTTTC
CCATCTAACCGAATCCATGAACCGATACCGGGAAGGGAAGGGAGACAAGCCCGGCC
GCGTGTTCCGTCCACACGTTGCGGACGTACTCAAGTTCTGCCGGCGAGCCGATGGC
GGAAAGCAGAAAGACGACCTGGTAGAAACCTGCATTCGGTTAAACACCACGCACGT
TGCCATGCAGCGTACGAAGAAGGCCAAGAACGGCCGCCTGGTGACGGTATCCGAGG
GTGAAGCCTTGATTAGCCGCTACAAGATCGTAAAGAGCGAAACCGGGCGGCCGGAG
TACATCGAGATCGAGCTAGCTGATTGGATGTACCGCGAGATCACAGAAGGCAAGAA
CCCGGACGTGCTGACGGTTCACCCCGATTACTTTTTGATCGATCCCGGCATCGGCC
GTTTTCTCTACCGCCTGGCACGCCGCGCCGCAGGCAAGGCAGAAGCCAGATGGTTG
TTCAAGACGATCTACGAACGCAGTGGCAGCGCCGGAGAGTTCAAGAAGTTCTGTTT
CACCGTGCGCAAGCTGATCGGGTCAAATGACCTGCCGGAGTACGATTTGAAGGAGG
AGGCGGGGCAGGCTGGCCCGATCCTAGTCATGCGCTACCGCAACCTGATCGAGGGC
GAAGCATCCGCCGGTTCCTAATGTACGGAGCAGATGCTAGGGCAAATTGCCCTAGC
AGGGGAAAAAGGTCGAAAAGGTCTCTTTCCTGTGGATAGCACGTACATTGGGAACC
CAAAGCCGTACATTGGGAACCGGAACCCGTACATTGGGAACCCAAAGCCGTACATT
GGGAACCGGTCACACATGTAAGTGACTGATATAAAAGAGAAAAAAGGCGATTTTTC
CGCCTAAAACTCTTTAAAACTTATTAAAACTCTTAAAACCCGCCTGGCCTGTGCAT
AACTGTCTGGCCAGCGCACAGCCGAAGAGCTGCAAAAAGCGCCTACCCTTCGGTCG
CTGCGCTCCCTACGCCCCGCCGCTTCGCGTCGGCCTATCGCGGCCGCTGGCCGCTC
AAAAATGGCTGGCCTACGGCCAGGCAATCTACCAGGGCGCGGACAAGCCGCGCCGT
CGCCACTCGACCGCCGGCGCCCACATCAAGGCACCCTGCCTCGCGCGTTTCGGTGA
TGACGGTGAAAACCTCTGACACATGCAGCTCCCGGAGACGGTCACAGCTTGTCTGT
AAGCGGATGCCGGGAGCAGACAAGCCCGTCAGGGCGCGTCAGCGGGTGTTGGCGGG
TGTCGGGGCGCAGCCATGACCCAGTCACGTAGCGATAGCGGAGTGTATACTGGCTT
AACTATGCGGCATCAGAGCAGATTGTACTGAGAGTGCACCATATGCGGTGTGAAAT
ACCGCACAGATGCGTAAGGAGAAAATACCGCATCAGGCGCTCTTCCGCTTCCTCGC
TCACTGACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAACATGTG
AGCAAAAGGCCAGCAAAAGGCCAGGAACCGTAAAAAGGCCGCGTTGCTGGCGTTTT
TCCATAGGCTCCGCCCCCCTGACGAGCATCACAAAAATCGACGCTCAAGTCAGAGG
TGGCGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCGGT
GCTACAGAGTTCTTGAAGTGGTGGCCTAACTACGGCTACACTAGAAGGACAGTATT
TGGTATCTGCGCTCTGCTGAAGCCAGTTACCTTCGGAAAAAGAGTTGGTAGCTCTT
GATCCGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGGGGTC
TGACGCTCAGTGGAACGAAAACTCACGTTAAGGGATTTTGGTCATGCATTCTAGGT
ACTAAAACAATTCATCCAGTAAAATATAATATTTTATTTTCTCCCAATCAGGCTTG
ATCCCCAGTAAGTCAAAAAATAGCTCGACATACTGTTCTTCCCCGATATCCTCCCT
GATCGACCGGACGCAGAAGGCAATGTCATACCACTTGTCCGCCCTGCCGCTTCTCC
CAAGATCAATAAAGCCACTTACTTTGCCATCTTTCACAAAGATGTTGCTGTCTCCC
AGGTCGCCGTGGGAAAAGACAAGTTCCTCTTCGGGCTTTTCCGTCTTTAAAAAATC
ATACAGCTCGCGCGGATCTTTAAATGGAGTGTCTTCTTCCCAGTTTTCGCAATCCA
CATCGGCCAGATCGTTATTCAGTAAGTAATCCAATTCGGCTAAGCGGCTGTCTAAG
CTATTCGTATAGGGACAATCCGATATGTCGATGGAGTGAAAGAGCCTGATGCACTC
CGCATACAGCTCGATAATCTTTTCAGGGCTTTGTTCATCTTCATACTCTTCCGAGC
AAAGGACGCCATCGGCCTCACTCATGAGCAGATTGCTCCAGCCATCATGCCGTTCA
AAGTGCAGGACCTTTGGAACAGGCAGCTTTCCTTCCAGCCATAGCATCATGTCCTT
TTCCCGTTCCACATCATAGGTGGTCCCTTTATACCGGCTGTCCGTCATTTTTAAAT
ATAGGTTTTCATTTTCTCCCACCAGCTTATATACCTTAGCAGGAGACATTCCTTCC
GTATCTTTTACGCAGCGGTATTTTTCGATCAGTTTTTTCAATTCCGGTGATATTCT
CATTTTAGCCATTTATTATTTCCTTCCTCTTTTCTACAGTATTTAAAGATACCCCA
AGAAGCTAATTATAACAAGACGAACTCCAATTCACTGTTCCTTGCATTCTAAAACC
TTAAATACCAGAAAACAGCTTTTTCAAAGTTGTTTTCAAAGTTGGCGTATAACATA
GTATCGACGGAGCCGATTTTGAAACCGCGGTGATCACAGGCAGCAACGCTCTGTCA
TCGTTACAATCAACATGCTACCCTCCGCGAGATCATCCGTGTTTCAAACCCGGCAG
CTTAGTTGCCGTTCTTCCGAATAGCATCGGTAACATGAGCAAAGTCTGCCGCCTTA
CAACGGCTCTCCCGCTGACGCCGTCCCGGACTGATGGGCTGCCTGTATCGAGTGGT
GATTTTGTGCCGAGCTGCCGGTCGGGGAGCTGTTGGCTGGCTGGTGGCAGGATATA
TTGTGGTGTAAACAAATTGACGCTTAGACAACTTAATAACACATTGCGGACGTTTT
TAATGTACTGAATTAACGCCGAATTAATTCGGGGGATCTGGATTTTAGTACTGGAT
TTTGGTTTTAGGAATTAGAAATTTTATTGATAGAAGTATTTTACAAATACAAATAC
ATACTAAGGGTTTCTTATATGCTCAACACATGAGCGAAACCCTATAGGAACCCTAA
TTCCCTTATCTGGGAACTACTCACACATTATTATGGAGAAACTCGAGCTTGTCGAT
CGACAGATCCGGTCGGCATCTACTCTATTTCTTTGCCCTCGGACGAGTGCTGGGGC
GTCGGTTTCCACTATCGGCGAGTACTTCTACACAGCCATCGGTCCAGACGGCCGCG
CTTCTGCGGGCGATTTGTGTACGCCCGACAGTCCCGGCTCCGGATCGGACGATTGC
GTCGCATCGACCCTGCGCCCAAGCTGCATCATCGAAATTGCCGTCAACCAAGCTCT
GATAGAGTTGGTCAAGACCAATGCGGAGCATATACGCCCGGAGTCGTGGCGATCCT
GCAAGCTCCGGATGCCTCCGCTCGAAGTAGCGCGTCTGCTGCTCCATACAAGCCAA
CCACGGCCTCCAGAAGAAGATGTTGGCGACCTCGTATTGGGAATCCCCGAACATCG
CCTCGCTCCAGTCAATGACCGCTGTTATGCGGCCATTGTCCGTCAGGACATTGTTG
GAGCCGAAATCCGCGTGCACGAGGTGCCGGACTTCGGGGCAGTCCTCGGCCCAAAG
CATCAGCTCATCGAGAGCCTGCGCGACGGACGCACTGACGGTGTCGTCCATCACAG
TTTGCCAGTGATACACATGGGGATCAGCAATCGCGCATATGAAATCACGCCATGTA
GTGTATTGACCGATTCCTTGCGGTCCGAATGGGCCGAACCCGCTCGTCTGGCTAAG
ATCGGCCGCAGCGATCGCATCCATAGCCTCCGCGACCGGTTGTAGAACAGCGGGCA
GTTCGGTTTCAGGCAGGTCTTGCAACGTGACACCCTGTGCACGGCGGGAGATGCAA
TAGGTCAGGCTCTCGCTAAACTCCCCAATGTCAAGCACTTCCGGAATCGGGAGCGC
GGCCGATGCAAAGTGCCGATAAACATAACGATCTTTGTAGAAACCATCGGCGCAGC
TATTTACCCGCAGGACATATCCACGCCCTCCTACATCGAAGCTGAAAGCACGAGAT
TCTTCGCCCTCCGAGAGCTGCATCAGGTCGGAGACGCTGTCGAACTTTTCGATCAG
AAACTTCTCGACAGACGTCGCGGTGAGTTCAGGCTTTTTCATATCTCATTGCCCCC
CGGGATCTGCGAAAGCTCGAGAGAGATAGATTTGTAGAGAGAGACTGGTGATTTCA
GCGTGTCCTCTCCAAATGAAATGAACTTCCTTATATAGAGGAAGGTCTTGCGAAGG
ATAGTGGGATTGTGCGTCATCCCTTACGTCAGTGGAGATATCACATCAATCCACTT
GCTTTGAAGACGTGGTTGGAACGTCTTCTTTTTCCACGATGCTCCTCGTGGGTGGG
GGTCCATCTTTGGGACCACTGTCGGCAGAGGCATCTTGAACGATAGCCTTTCCTTT
ATCGCAATGATGGCATTTGTAGGTGCCACCTTCCTTTTCTACTGTCCTTTTGATGA
AGTGACAGATAGCTGGGCAATGGAATCCGAGGAGGTTTCCCGATATTACCCTTTGT
TGAAAAGTCTCAATAGCCCTTTGGTCTTCTGAGACTGTATCTTTGATATTCTTGGA
GTAGACGAGAGTGTCGTGCTCCACCATGTTATCACATCAATCCACTTGCTTTGAAG
ACGTGGTTGGAACGTCTTCTTTTTCCACGATGCTCCTCGTGGGTGGGGGTCCATCT
TTGGGACCACTGTCGGCAGAGGCATCTTGAACGATAGCCTTTCCTTTATCGCAATG
ATGGCATTTGTAGGTGCCACCTTCCTTTTCTACTGTCCTTTTGATGAAGTGACAGA
TAGCTGGGCAATGGAATCCGAGGAGGTTTCCCGATATTACCCTTTGTTGAAAAGTC
TCAATAGCCCTTTGGTCTTCTGAGACTGTATCTTTGATATTCTTGGAGTAGACGAG
AGTGTCGTGCTCCACCATGTTGGCAAGCTGCTCTAGCCAATACGCAAACCGCCTCT
CCCCGCGCGTTGGCCGATTCATTAATGCAGCTGGCACGACAGGTTTCCCGACTGGA
AAGCGGGCAGTGAGCGCAACGCAATTAATGTGAGTTAGCTCACTCATTAGGCACCC
CAGGCTTTACACTTTATGCTTCCGGCTCGTATGTTGTGTGGAATTGTGAGCGGATA
ACAATTTCACACAGGAAACAGCTATGACCATGATTACGAATTCCCGATCTAGTAAC
ATAGATGACACCGCGCGCGATAATTTATCCTAGTTTGCGCGCTATATTTTGTTTTC
TATCGCGTATTAAATGTATAATTGCGGGACTCTAATCATAAAAACCCATCTCATAA
ATAACGTCATGCATTACATGTTAATTATTACATGCTTAACGTAATTCAACAGAAAT
TATATGATAATCATCGCAAGACCGGCAACAGGATTCAATCTTAAGAAACTTTATTG
CCAAATGTTTGAACGATCGGGGAAATTCGAGCTCTCATGCATATTTAGTCGCTATC
TGCTGGAGGGAATCGAATAGTTCTTGTGGCTTGCAAAGCATAACCATATGATCTGC
GTCTTTGACCTCTACAACGTCATTGAACCCAACATTTTGGATCATCAAGAGCTGAT
ATTCCAATGGAATTCCAAGGTCCTCCGTGCAAACAATAAAGGCACGTGGAACTGAC
CCATATCCCTCTTTGGAGAAGTTCTTTTGTTTAGTCAAGTCTTCCATGAAGAGCGA
TGATGGCCTTGCTAAAGTCTTGGCCAATTCCAAGTCCTCAATAGGGGACAGTTGGT
AGAGCTTGTCGGACAAGAAGTTGGGGCCGAAGAACATTGATGTTTTGTTTCCGCTT
GGAGCAAATTCAGTGTCTAACCATGCAGCTAACGGGGTCCTCTCATTGTACTTTTC
CAAGACATAAGATGGGTGGTGTTCAGTGTCTGGAGCAAAAGCTGTTAAGAAAACAC
CAACTGCTACCTTTTCTGGGAATTTCTCCATTGCAAGTGCTATGTTCAGCCCTCCA
AGGCTGTGACCAACTAGAACTAACTTCTCATTTGAGGGAATTGTGGCCATTAGCTG
CAACAAAGGCGCAGAATACTCTGAGAAAGTATCAACATCTTCAATTTTCTTCATGT
TGGTTCCAGAAGCTGCAAGGTCAAGTACTGTGACCTTATGGCCTGCAGATTCCAAG
CGTGGCTTGAGCTTATACCAACACCAAGCTCCATGGCATGCCCCATGCACCAGAAC
ATAGTGCTTCCTATCCATACAATTTTGTGAACCCATGGATCCTCTAGAGTCCCCCG
TGTTCTCTCCAAATGAAATGAACTTCCTTATATAGAGGAAGGGTCTTGCGAAGGAT
AGTGGGATTGTGCGTCATCCCTTACGTCAGTGGAGATATCACATCAATCCACTTGC
TTTGAAGACGTGGTTGGAACGTCTTCTTTTTCCACGATGCTCCTCGTGGGTGGGGG
TCCATCTTTGGGACCACTGTCGGCAGAGGCATCTTCAACGATGGCCTTTCCTTTAT
CGCAATGATGGCATTTGTAGGAGCCACCTTCCTTTTCCACTATCTTCACAATAAAG
TGACAGATAGCTGGGCAATGGAATCCGAGGAGGTTTCCGGATATTACCCTTTGTTG
AAAAGTCTCAATTGCCCTTTGGTCTTCTGAGACTGTATCTTTGATATTTTTGGAGT
AGACAAGTGTGTCGTGCTCCACCATGTTGACGAAGATTTTCTTCTTGTCATTGAGT
CGTAAGAGACTCTGTATGAACTGTTCGCCAGTCTTTACGGCGAGTTCTGTTAGGTC
CTCTATTTGAATCTTTGACTCCATGGCCTTTGATTCAGTGGGAACTACCTTTTTAG
AGACTCCAATCTCTATTACTTGCCTTGGTTTGTGAAGCAAGCCTTGAATCGTCCAT
ACTGGAATAGTACTTCTGATCTTGAGAAATATATCTTTCTCTGTGTTCTTGATGCA
GTTAGTCCTGAATCTTTTGACTGCATCTTTAACCTTCTTGGGAAGGTATTTGATTT
CCTGGAGATTATTGCTCGGGTAGATCGTCTTGATGAGACCTGCTGCGTAAGCCTCT
CTAACCATCTGTGGGTTAGCATTCTTTCTGAAATTGAAAAGGCTAATCTGGGGACC
TGCAGGCATGCAAGCTTGGCACTGGCCGTCGTTTTACAACGTCGTGACTGGGAAAA
CCCTGGCGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGCCTGAAT
GGCGAATGCTAGAGCAGCTTGAGCTTGGATCAGATTGTCGTTTCCCGCCTTCAGTT
TAGCTTCATGGAGTCAAAGATTCAAATAGAGGACCTAACAGAACTCGCCGTAAAGA
CTGGCGAACAGTTCATACAGAGTCTCTTACGACTCAATGACAAGAAGAAAATCTTC
GTCAACATGGTGGAGCACGACACACTTGTCTACTCCAAAAATATCAAAGATACAGT
CTCAGAAGACCAAAGGGCAATTGAGACTTTTCAACAAAGGGTAATATCCGGAAACC
TCCTCGGATTCCATTGCCCAGCTATCTGTCACTTTATTGTGAAGATAGTGGAAAAG
GAAGGTGGCTCCTACAAATGCCATCATTGCGATAAAGGAAAGGCCATCGTTGAAGA
TGCCTCTGCCGACAGTGGTCCCAAAGATGGACCCCCACCCACGAGGAGCATCGTGG
AAAAAGAAGACGTTCCAACCACGTCTTCAAAGCAAGTGGATTGATGTGATATCTCC
ACTGACGTAAGGGATGACGCACAATCCCACTATCCTTCGCAAGACCCTTCCTCTAT
ATAAGGAAGTTCATTTCATTTGGAGAGAACACGGGGGACTCTTGAC

C. Transgenics

An expression cassette including a disclosed a nucleic acid sequence encoding GmSAMT1 (such as set forth in SEQ ID NO: 1 or a degenerate variant thereof) and/or GmSABP2-1 such as set forth in SEQ ID NO: 2) or functional fragment thereof, operably linked to promoter and optionally other heterologous nucleic acids can be used to transform any plant, for example as a vector, such as a plasmid. In this manner, genetically modified plants, plant cells, plant tissue, seed, and the like can be obtained. Thus, methods of producing a plant resistant to a pest are disclosed. Such methods, include introducing into a plant GmSAMT1 (such as set forth in SEQ ID NO: 1 or a degenerate variant thereof) and/or GmSABP2-1 such as set forth in SEQ ID NO: 2 or a degenerate variant thereof) or functional fragment thereof, operably linked to promoter and optionally other heterologous nucleic acids. Also disclosed are methods increasing pest resistance in a plant. Such methods, include introducing into a plant GmSAMT1 (such as set forth in SEQ ID NO: 1 or a degenerate variant thereof) and/or GmSABP2-1 such as set forth in SEQ ID NO: 2 or a degenerate variant thereof) or functional fragment thereof, operably linked to promoter and optionally other heterologous nucleic acids, thereby increasing pest resistance in the plant. The plant can be transiently or stably transformed. Pest resistance, such as an increase in pest resistance can be determined relative to a relevant control plant, such as a plant that does not express a pest resistance polypeptide disclosed herein, a plant that has not been transformed with a nucleic acid encoding a pest resistance polypeptide as disclosed herein, a plant that is transformed with an irrelevant nucleic acid, and the like. The control plant is generally matched for species, variety, age, and the like and is subjected to the same growing conditions, for example temperature, soil, sunlight, pH, water, and the like. The selection of a suitable control plant is routine for those skilled in the art.

Transformation protocols as well as protocols for introducing nucleotide sequences into plants may vary depending on the type of plant or plant cell, for example, monocot or dicot, targeted for transformation. Suitable methods of introducing nucleotide sequences into plant cells and subsequent insertion into the plant genome include microinjection (Crossway et al., Biotechniques 4:320-334, 1986), electroporation (Riggs et al., Proc. Natl. Acad. Sci. USA 53:5602-5606, 1986), Agrobacterium-mediated transformation (U.S. Pat. No. 5,563,055), direct gene transfer (Paszkowski et al., EMBO J. 3:2717-2722, 1984), and ballistic particle acceleration (see, for example, U.S. Pat. No. 4,945,050; Tomes et al. “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin), 1995; and McCabe et al., Biotechnology 5:923-926, 1988). Also see Weissinger et al., Ann. Rev. Genet. 22:421-477, 1988; Sanford et al., Paniculate Science and Technology 5:27-37, 1987; Christou et al., Plant Physiol 57:671-674, 1988; McCabe et al., Bio/Technology 5:923-926, 1988; Finer and McMullen, In Vitro Cell Dev. Biol. 27P:175-182, 1991; Singh et al., Theor. Appl Genet. 95:319-324, 1998; Datta et al., Biotechnology 5:736-740, 1990; Klein et al., Proc. Natl. Acad. Sci. USA 55:4305-4309, 1988; Klein et al., Biotechnology 5:559-563, 1988; U.S. Pat. Nos. 5,240,855, 5,322,783 and 5,324,646; Klein et al., Plant Physiol 97:440-444, 1988; Fromm et al. Biotechnology 5:833-839, 1990; Hooykaas-Van Slogteren et al., Nature 377:763-764, 1984; Bytebier et al., Proc. Natl. Acad. Sci. USA 54:5345-5349, 1987; De Wet et al. (1985) in The Experimental Manipulation of Ovule Tissues, ed. Chapman et al. (Longman, New York), pp. 197-209; Kaeppler et al., Plant Cell Reports 9:415-418, 1990; Kaeppler et al., Theor. Appl. Genet. 54:560-566, 1992; D'Halluin et al., Plant Cell 4:1495-1505 1992; Li et al., Plant Cell Reports 72:250-255, 1993; Christou and Ford Annals of Botany 75:407-413, 1995; Osjoda et al., Nature Biotechnology 74:745-750, 1996; and the like. “Introducing” in the context of a plant cell, plant tissue, plant part and/or plant means contacting a nucleic acid molecule with the plant cell, plant tissue, plant part, and/or plant in such a manner that the nucleic acid molecule gains access to the interior of the plant cell or a cell of the plant tissue, plant part or plant. Where more than one nucleic acid molecule is to be introduced, these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, for example as part of a breeding protocol.

The cells that have been transformed may be grown into plants in accordance with conventional ways. See, for example, McCormick et al. Plant Cell Reports 5:81-84, 1986. These plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

The pest resistance genes disclosed herein, such as nucleic acid sequences encoding GmSAMT1 (such as set forth in SEQ ID NO: 1 or a degenerate variant thereof) and/or GmSABP2-1 such as set forth in SEQ ID NO: 2 or a degenerate variant thereof) or active variant and fragments thereof may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. In particular embodiments, the pest resistance genes disclosed herein, such as nucleic acid sequences encoding GmSAMT1 (such as set forth in SEQ ID NO: 1 or a degenerate variant thereof) and/or GmSABP2-1 such as set forth in SEQ ID NO: 2 or a degenerate variant thereof) or active variant and fragments thereof may be used for transformation of soybean (Glycine max).

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis), and Poplar and Eucalyptus.

In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments soybean plants are optimal. Other plants of interest include grain plants that provide seeds of interest, oilseed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

In some embodiments, the polynucleotides comprising disclosed pest resistance gene are engineered into a molecular stack. Thus, the various plants, plant cells and seeds disclosed herein can further comprise one or more traits of interest, and in more specific embodiments, the plant, plant part or plant cell is stacked with any combination of polynucleotide sequences of interest in order to create plants with a desired combination of traits. As used herein, the term “stacked” includes having the multiple traits present in the same plant.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853.

The transformed plants may be analyzed for the presence of the gene(s) of interest and the expression level. Numerous methods are available to those of ordinary skill in the art for the analysis of transformed plants. For example, methods for plant analysis include Southern and northern blot analysis, PCR-based (or other nucleic acid amplification-based) approaches, biochemical analyses, phenotypic screening methods, field evaluations, and immunodiagnostic assays (e.g., for the detection, localization, and/or quantification of proteins).

### EXAMPLES

### Example 1

### Isolation and Characterization of GmSAMT1

Plant, Nematode, Bacterium, and Chemical Sources:

In the studies described herein, two genetically-related soybean lines, TN02-226 and TN02-275, were used for gene cloning and generating soybean hairy roots. The breeding details for these two soybean lines have been described in Mazarei et al. Theor. Appl. Genet. 123: 1193-1206, 2011, which is specifically incorporated herein by reference in its entirety. These two F₆-derived sister lines, TN02-226 and TN02-275, are resistant and susceptible, respectively, to the SCN race 2 (HG type 1.2.5.7), which has been confirmed in USDA Southern Regional Tests from 2004 through 2007. In the studies described herein, the SCN race 2 eggs were utilized as the pathogens in the bioassay of the transgenic hairy roots since the above two soybean lines exhibited different responses to SCN race 2. The maintenance of SCN followed the method described by Arelli et al., Heterodera glycines. Crop Sci. 40: 824-826, 2000. Escherichia coli strain BL21 (DE3) CODONPLUS® (Stratagene, La Jolla, Calif.) was used for the expression of recombinant protein. Agrobacterium cultures, A. tumefaciens GV3101 and A. rhizogenes strain K599, were utilized for tobacco transient transformation and the generation of the soybean hairy roots. All chemicals were obtained from Sigma-Aldrich (St. Louis, Mo.).

Viruses, Soybean Genotypes, Inoculation, and SMV Detection:

In the studies described herein, a mutant of soybean mosaic virus SMV-N (SMV-N25) was used for virus infection on soybean. Two soybean genotypes Williams 82, susceptible to SMV-N25, and L78-375, resistant to SMV-N25, were used. The mechanically inoculated plants were maintained in a growth chamber operating at 22° C. with a photoperiod of 16 h. SMV inoculated soybean seedlings were grown for another 21 days and their trifoliate leaves were harvested, pulverized in the presence of liquid nitrogen and stored at −80° C. until processed for quantitative reverse-transcription PCR analysis.

Sequence Analysis:

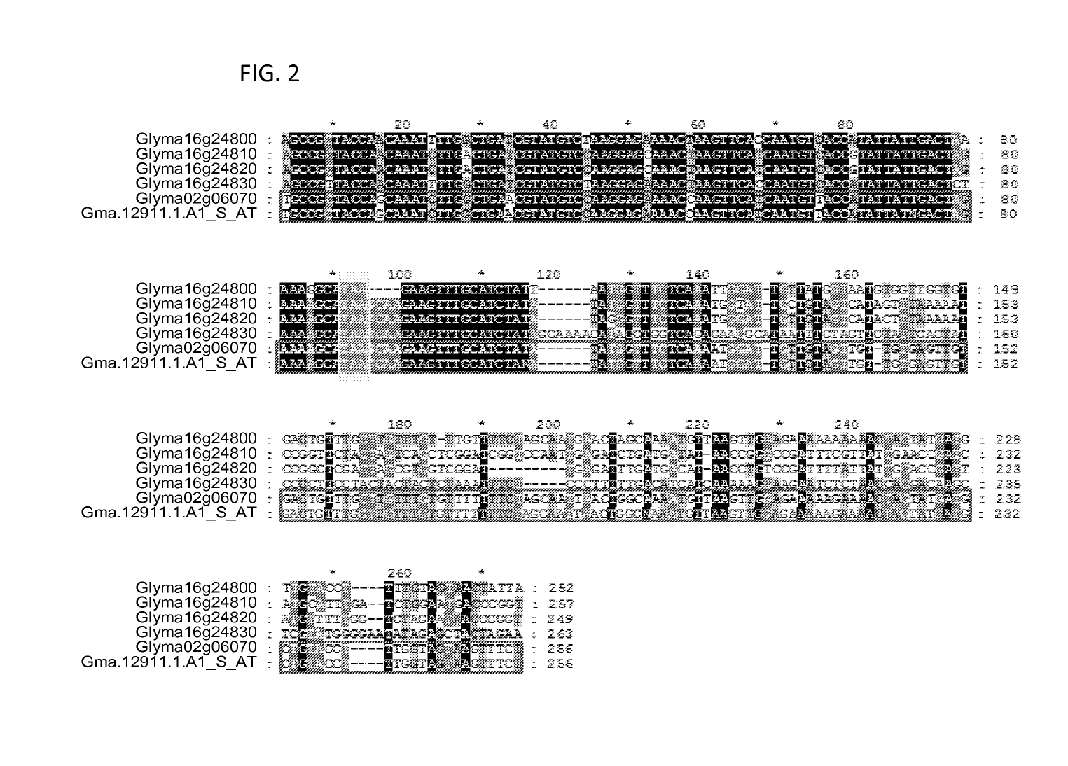

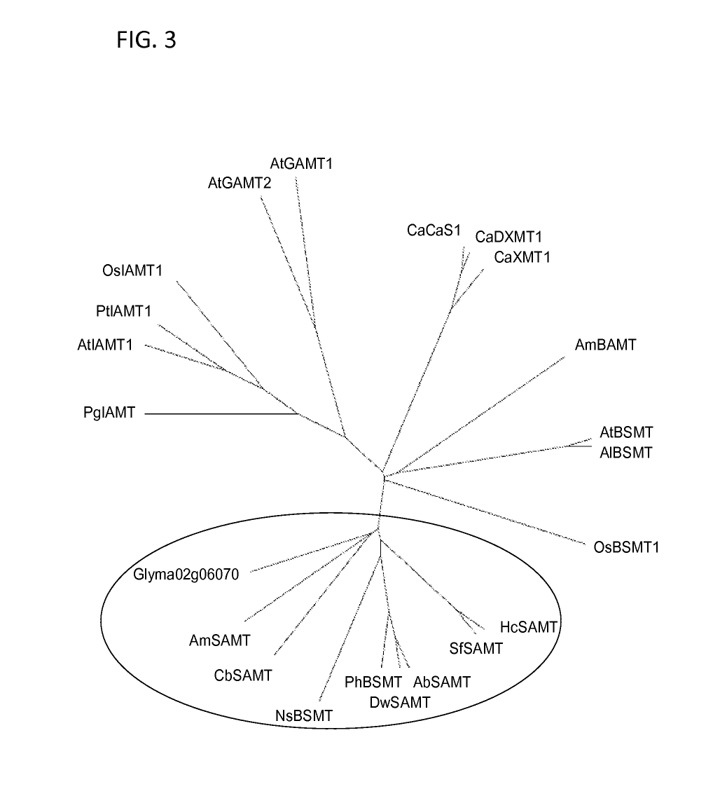

The sequence of Affymetrix probe Gma.12911.1.A1_S_AT, which identified our target gene in microarray experiment, was used as a query to BLAST search against the soybean database GmGDB/GMtranscript (as available on the world wide web at plantgdb.org/GmGDB/cgi-bin/blastGDB.p1). Program blastn was performed with E-value of 1e−20. Five soybean genes showed high similarity (FIG. 2). A multiple sequence alignment of selected SABATH proteins and Glyma02g06070 was performed using ClustalX program and viewed using TreeView software (as available on the world wide web at taxonomy.zoology.gla.ac.uk/rod/treeview.html). A neighbor-joining unrooted phylogenetic tree was constructed (FIG. 3).

Isolation of Glyma02g06070 Gene from Soybean:

Total RNA was isolated from the SCN-infected the root tissue of soybean line TN02-226, using the RNEASY® Plant Mini Kit (QIAGEN®, Valencia, Calif., USA) and DNA contamination was removed with an on-column DNase (QIAGEN®, Valencia, Calif., USA) treatment. Then cDNA was synthesized from total RNA in a 15 μl reaction volume using the First-Strand cDNA Synthesis Kit (GE Healthcare, Piscataway, N.J., USA) as previously described. Based on microarray analysis, one soybean candidate gene Glyma02g06070 was chosen for further study. The full length cDNA sequence of Glyma02g06070 was gained from public soybean database (as available on the world wide web at phytozome.net/soybean), and primers were designed as following ATGGAAGTAGCACAGGTACTCCACATG (SEQ ID NO: 5) and TGCTTTTCTAGTCAATAATATGGTAAC (SEQ ID NO: 6). The PCR conditions were as follows: 94° C. for 2 min followed by 35 cycles at 94° C. for 30 s, 57° C. for 30 s and 72° C. for 1 min 30 s, and a final extension at 72° C. for 10 min. PCR products were cloned into vector pEXP5/CT-TOPO®. By sequencing, the PCR product from TN02-226 soybean was the same as the one predicted in the soybean database.

To verify its biochemical function, the candidate gene was then cloned into a protein expression construct vector, pET100/D-TOPO® vector (Invitrogen, Carlsband, Calif.) and expressed in the E. coli strain BL21 (DE3) CODONPLUS® (Stratagene, La Jolla, Calif.). Protein expression was induced by isopropyl β-D-1-thiogalactopyranoside (IPTG) at a concentration of 500 μM for 18 h at 25° C., with cells lysed by sonication. E. coli-expressed Glyma02g06070 with a His-tag was purified from the E. coli cell lysate using Ni-NTA agarose following the manufacturer instructions (Invitrogen). Protein purity was verified by SDS-PAGE and protein concentrations were determined by the Bradford assay.

Radiochemical SAMT Activity Assay:

A 50 μl volume containing 50 mM Tris-HCl, pH 7.5, 1 mM SA, and 3 μM ¹⁴C-SAM with a specific activity of 51.4 mCi/mmol (Perkin-Elmer, Boston, Mass.) was used. The assay was initiated by addition of SAM, maintained at 25° C. for 30 min, and stopped by the addition of ethyl acetate (150 μl). After phase separation by 1 min centrifugation at 14,000 g, the upper organic phase was counted using a liquid scintillation counter (Beckman Coulter, Fullerton, Calif.). The radiation counts in the organic phase indicated the amount of synthesized MeSA. Substrate specificity assay was also performed for recombinant GmSAMT1, with a range of substrates including SA, benzoic acid, anthranilic acid, 3-hydroxybenzoic acid, 4-hydroxybenzoic acid, nicotinic acid, p-coumaric acid, caffeic acid, cinnamic acid, vanallic acid, jasmonic acid (JA), gibberellic acid, indole-3-acetic acid, and 2,4-dichlorophenoxyacetic acid. The activity with SA was set at 100%. Three independent assays were performed for each compound.

Determination of Kinetic Parameters of GmSAMT I:

Appropriate enzyme concentrations and incubation time were determined in time course assays to make sure that the reaction velocity was linear during the assay period. Lineweaver-Burk plots yielded apparent Km values. To determine the Km for SA, concentrations of SA were independently varied from 2 to 150 μM, while SAM was held constant at 200 μM. Assays were conducted at 25° C. for 30 min, as described in SAMT activity assay. The optimal pH for GmSAMT1 was assayed from pH 6.0 to pH 9.0, and optimal reaction of temperature was assayed ranging from 0° C. to 60° C. Effects of metal ions on activity of GmSAMT1 were assayed with a range of metal ions, including K⁺, Cu²⁺, Zn²⁺, Fe²⁺, Fe³⁺, Mn²⁺, Ca²⁺, Na⁺, NH₄⁺, and Mg²⁺, in the form of chloride salts at 5 mM final concentrations.

Creating the Constructs with Over-expressed GmSAMT1 and OFP Reporter Genes:

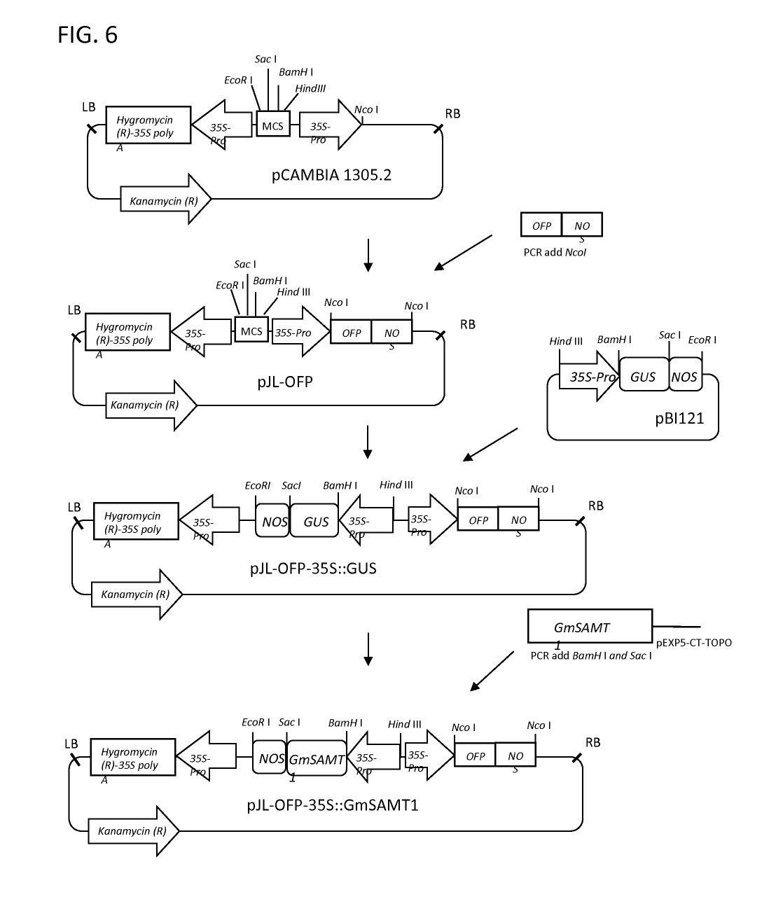

The pCAMBIA 1305.2 vector was used as a primary binary vector. The following is the procedures of constructing the vector harboring over-expressed GmSAMT1 (FIG. 6). As a screening tool, a orange fluorescent protein (OFP) gene, ppor RFP with primers containing NcoI fragment, was introduced into NcoI site of pCAMBIA 1305.2 vector. This modified pCAMBIA 1305.2 vector was called pJL-OFP vector and used to test OFP expression through transient transforming tobacco. Then the cassette of 35S::GUS/NOS terminator was taken from a pBI121 vector by digestion with EcoRI and HindIII and inserted into the EcoRI and HindIII sites of pJL-OFP vector to form pJL-OFP-35S::GUS vector, which was used for testing the OFP and GUS gene expression in one binary vector. GmSAMT1 gene was amplified from the above mentioned pEXP5/CT-TOPO® vector with primers containing BamHI and Sad fragment and replaced with GUS gene in pJL-OFP-35S::GUS vector by digestion and ligation. Finally the over-expressed GmSAMT1 vector was constructed and called pJL-OFP-35S::GmSAMT1 vector. The vectors of pJL-OFP-35S::GmSAMT1 and pJL-OFP were transformed into A. rhizogenes as target gene and vector control, respectively. A. tumefaciens GV3101 and A. rhizogenes strain K599, were transformed by freeze-thaw method. The cells with no binary vector were plated on LB media with no antibiotic. The cells with the binary vectors were plated on selective LB media with kanamycin and incubated at 28° C. for 2 days.

Testing the Reporter Gene Expression Through Tobacco Transient Transformation:

Before generating hairy roots, the constructs containing OFP gene were tested by transient expression assays in tobacco plants according to Sparkes et al., Nat. Protocols 1: 2019-2025, 2006. The plasmids, pJL-OFP and pJL-OFP-35S::GmSAMT1, were tested for OFP function by agroinfiltration of A. tumefaciens GV3101 into tobacco leaves, along with A. tumefaciens GV3101 without binary plasmid as a negative control. Bacterial suspensions carrying target plasmids were pressure-infiltrated using a syringe with no needle into the 5-week tobacco leaves through the abaxial surfaces over leaf segments lying between the major veins that branch off from the mid-rib of the leaf OFP fluorescence was investigated 2 days after infiltration in excised leaf sections that covered the infiltrated zone part of surrounding non-infiltrated tissue, using an Olympus SZX12 fluorescence microscope and an OFP filter.

Generation of Transgenic Soybean Hairy Roots:

The soybean hairy root transformation system by infection with A. rhizogenes harboring the target genes were modified and used to study soybean resistance against SCN. A. rhizogenes K599 harboring the target gene, vector control and A. rhizogenes K599 alone were used to inoculate the TN02-275 soybeans, which are susceptible to SCN race 2. To better evaluate the resistance of the target gene, A. rhizogenes harboring the vector control and A. rhizogenes K599 alone were also used to inoculate the TN02-226 soybeans, which are resistant to SCN race 2. Generation of soybean hairy roots was modified as follows: First, soybean seeds were sterilized with chlorine gas. Second, a single layer of mature soybean seeds with intact seed coats were placed in open Petri dish, which was put in a bell jar desiccator within a fume hood. A beaker with 100 ml bleach was also placed in the desiccator and added 5 ml concentrated (12N) HCl along the side of the beaker. Then the seeds were kept in the closed desiccator immediately and maintained in the chlorine gas atmosphere overnight (about 16 h). After sterilization, seeds were transferred to the autoclaved filter paper moistened with sterilized distilled water in the Petri dishes for germination in a laminar flow hood. The germinated soybean seeds were sowed in sterilized vermiculite and six seeds were grown in each cell of 18-cell germination tray. The overnight cultured bacteria lawn of A. rhizogenes K599 with binary vector pJL-OFP, pJL-OFP-35S::GUS or pJL-OFP-35S::GmSAMT1 from LB medium plate containing 50 mg ml⁻¹ kanamycin, or a culture of A. rhizogenes K599 lacking the binary vector grown in LB medium without any antibiotics was collected and suspended in 1 ml of sterile distilled water, respectively. Soybean cotyledonary nodes and upper hypocotyls of 5-day-old seedlings with unfolded cotyledons were stabbed three times by the syringe containing the bacterial suspension. In the first week, the trays were covered with the sterile transparent lids to retain high humidity. In the subsequent three weeks, plants were transferred into pots and the A. rhizogenes wounding sites were covered by wet sterile vermiculite to continually keep high humidity. Each day soybean plants were watered using sterile B&D solution containing 1 mM CaCl₂, 0.5 mM KH₂PO₄, 10 μM Fe-citrate, 0.25 mM MgSO₄, 0.25 mM K₂SO₄, 1 μM MnSO₄, 2 μM H₃BO₄, 0.5 μM ZnSO₄, 2 μM CuSO₄, 0.1 μM CoSO₄, 0.1 μM Na₂MoO₄, and 1 mM KNO₃. The growth conditions for soybean plants were 12 h light/12 h dark at 28° C./25° C. with irradiance from fluorescent bulbs at 150-200 μmol m⁻²·s⁻¹. After about four weeks, the hairy roots grew to approximately 10 cm in length. Soybean transgenic roots were detected based on OFP expression, using a fluorescence stereomicroscope (Olympus SZX12 fluorescence microscope and OFP filter). The tap root and non-transgenic roots were excised under the wounding site. Transgenic roots harboring pJL-OFP-35S::GUS screened by OFP signal were immersed in GUS solution (1 mM X-Gluc), 0.5 mM potassium ferrocyanide, 0.1% (v/v) TRITON™ X-100, 100 mM sodium phosphate buffer pH 7.0), and incubated at 37° C. overnight. In the following day, the X-Gluc solution was discarded. The roots were washed repeatedly with 70% ethanol to remove chlorophyll. Transgenic roots with visual GUS stain were recorded.

Nematode Infection and Demographics Assay:

To easily compare the SCN infection, one transgenic root was left for the SCN inoculation. Other transgenic roots were collected for RNA extraction and detection of the gene expression. The hairy root parts of twenty soybean plants harboring the same construct were loaded horizontally in a 13×9×2 cm sterilized inoculating tray containing one thin layer mixture of sterile sand and top soil (1:1) and the shoot parts were left out of inoculating tray About 0.75 ml of inoculum, which contained about 5600 SCN eggs, was added to each root system. Nematodes were allowed to infect roots for 7 days under humid conditions. Then all the roots were washed to remove extra SCN eggs and juvenile nematodes that had not penetrated the root tissue. Sterile vermiculite within cone-tainers was used for growing the infected chimeras in the growth chamber. In two weeks post inoculation, infected root samples were washed to remove vermiculite by tap water and cleared by 20% (v/v) bleach for no more than 4 min, and then stained by acid fuchsin. Nematodes of different stages within each infected root sample were recorded. Since the responses between the susceptible and resistant soybean lines occur on the J3 stage of SCN, the ratio of the number of J3+J4 to total number of nematodes per plant was used as an index to determine the resistance difference among the hairy roots harboring respective constructs. A minimum of 10 transformed plants were analyzed for each vector.

Quantitative Reverse Transcription Polymerase Chain Reaction (qRT-PCR):

Quantitative reverse transcription PCR was performed to check GmSAMT1 and some defense-related genes' transcription level of transgenic soybean hairy roots according to Mazarei et al., 2007. In order to conduct qRT-PCR assays, gene specific primers of GmSAMT1, defense-related genes, GmPR-1 (GenBank accession BU577813), GmPR-3 (GenBank accession AF202731), and reference gene, soybean ubiqutin3 gene (GmUBI-3, GenBank accession D28123), were designed (Table 1).

TABLE 1
Primers for qRT-PCR analysis of soybean SA methyltrans-
ferase gene and relevant pathogenesis-related genes.

| Gene Name | Orientation | Sequence (5′-3′) |
|---|---|---|
| GmSAMT1 | Forward | GTGAAATTGGAAGTTCTTAAAGAAGGA |
| | | (SEQ ID NO: 7) |
| | Reverse | CAGATTCAAAGTCTAGAGCATTCCAC |
| | | (SEQ ID NO: 8) |
| GmUBI-3 | Forward | GTGTAATGTTGGATGTGTTCCC |
| | | (SEQ ID NO: 9) |
| | Reverse | ACACAATTGAGTTCAACACAAACCG |
| | | (SEQ ID NO: 10) |
| GmPR-1 | Forward | AACTATGCTCCCCCTGGCAACTATATTG |
| | | (SEQ ID NO: 11) |
| | Reverse | TCTGAAGTGGTAGCTTCTACATCGAAACAA |
| | | (SEQ ID NO: 12) |
| GmPR-3 | Forward | AACTACAATTACGGGCAAGCTGGCAA |
| | | (SEQ ID NO: 13) |
| | Reverse | TTGATGGCTTGTTTCCCTGTGCAGT |
| | | (SEQ ID NO: 14) |
| GmNPR1-1 | Forward | AATTGACCAAGAGCTTCCGC |
| | | (SEQ ID NO: 26) |
| | Reverse | CTACAGAAGCATCATTTTCAACATCTT |
| | | (SEQ ID NO: 27) |
| GmNPR1-2 | Forward | CAATTGACCAAGAGCTTCCAA |
| | | (SEQ ID NO: 28) |
| | Reverse | CTATAGAAGCATCGTTTTCAACATCTC |
| | | (SEQ ID NO: 29) |
| GmICS1 | Forward | GGCCATTTCGGAGCTGG |
| | | (SEQ ID NO: 30) |
| | Reverse | AGGAGAAGGTGGTTCTGTGAGAGA |
| | | (SEQ ID NO: 31) |
| GmICS2 | Forward | CATGGCCATTTCGGAGCTTA |
| | | (SEQ ID NO: 32) |
| | Reverse | CAGAAGATGGTTCTGCGAATGG |
| | | (SEQ ID NO: 33) |

Total RNA was isolated from the respective root tissues of transgenic hairy roots and control hairy roots using RNEASY® columns (QIAGEN®) following the manufacturer's instructions for plants. RNA was treated with RNase-Free DNase set (QIAGEN®) to remove genomic DNA and total RNA (about 1.5 μg) was reverse-transcribed to synthesize cDNA using High-Capacity cDNA Reverse Transcription Kit (APPLIED BIOSYSTEMS®) for qRT-PCR according to manufacturer's instructions. First-strand cDNA was diluted and placed in each qRT-PCR reaction. DNA accumulation was measured using SYBR® Green as the reference dye. Genomic DNA contamination was monitored by PCR using RNA sample as template. Only one product was present in each reaction as indicated by the reference dye's dissociation curve of amplified products. The following PCR conditions were used: 50° C. for 2 min; 95° C. for 10 min; followed by 40 cycles of 95° C. for 15 s; 60° C. for 1 min; 72° C. for 30 s. All qRT-PCR assays were conducted in triplicate. PCR efficiencies for target and reference genes were equal between the target and control samples. Ct values and relative abundance were calculated using software supplied with the APPLIED BIOSYSTEMS® 7900 HT Fast Real-Time PCR system.

Statistical Analysis:

The resistance difference among the transgenic soybean hairy roots with over-expression of GmSAMT1 and control soybean hairy roots was analyzed by one way ANOVA using the mixed model of SAS (SAS 9.2 version). Least squares means of the resistance index were separated using Fisher's least significant difference (LSD) test. The expression difference of target gene and defense-related genes examined by qRT-PCR was also analyzed by one way ANOVA using the mixed model of SAS. Least squares means of relative expression level were separated using Fisher's LSD test. A P<0.05 was considered significant.

### Results

Glyma02g06070 is a Member of the SABATH Family:

Microarray analysis on soybean recombinant inbred lines (RILs), TN02-226 and TN02-275, revealed some genes with significant expression changes during resistant and susceptible reactions to SCN. One of these genes showed significant up-regulation only in SCN inoculated resistant soybean roots while no significant change of its gene expression occurred in SCN-inoculated susceptible soybean roots (Mazarei et al., Theor. Appl. Genet. 123: 1193-1206, 2011). To obtain the complete open reading frame of this resistance candidate gene, the sequence of Affymetrix SoyChip probe Gma.12911.1.A1_S_AT, which identified our target gene from microarrays, was used as a query to BLAST search against the soybean database GmGDB/GMtranscript (as available on the world wide web at plantgdb.org/GmGDB/cgi-bin/blastGDB.pl) and SoyBase (as available on the world wide web at soybase.org/gbrowse/cgi-bin/gbrowse/gmax1.01/), the soybean gene, Glyma02g06070 was found to have the highest similarity (99%) with the sequence of Gma.12911.1.A1_S_AT and was annotated for encoding SAM-dependent carboxyl methyltransferase (FIG. 2).

By further comparison with the known methyltransferases from other species, this SAM-dependent carboxyl methyltransferase encoded by Glyma02g06070 was determined to be a member of the SABATH family. The SABATH family is named after the first identified enzymes (SAMT, BAMT and theobromine synthase) in this family (Chen et al. 2003a). To investigate the evolutionary relationships between Glyma02g06070 and the known SABATH members from other species, a phylogenetic tree of selected functionally characterized SABATH members with Glyma02g06070 was constructed. The result showed that Glyma02g06070 belonged to the cluster containing the enzymes with salicylic acid methyltransferase activity (FIG. 3).

Glyma02g06070 Encodes Soybean SAMT:

A protein BLAST search reveals that Glyma02g06070 contains 370 amino acids and is 40.1% identical to the previously identified rice SAMT, OsBSMT1, at the amino acid sequence level. Glyma02g06070 was also predicted to contain an SAM-binding domain. When the sequence of Glyma02g06070 was searched in EST database, it also showed this gene is induced by SCN and SA. To confirm the prediction from the databases, we amplified and sequenced the complete open reading frame (ORF) of this gene from cDNA attained from SCN-infected root tissue of soybean TN02-226 line.

Since Glyma02g06070 encodes a member of the SABATH family, whose member enzymes can transfer a methyl group from methyl-donor SAM to a range of substrates, the enzyme activity of the recombinant protein of Glyma02g06070 was examined using various potential substrates (Table 2).

TABLE 2
Relative activity of GmSAMT1 with salicylic
acid and other related substrates

| Substrate | Relative activity^{a} (%) |
|---|---|
| Salicylic acid | 100 |
| Benzoic acid | 16.9 |
| Anthranilic acid | 9.3 |
| 3-Hydroxybenzoic acid | 1.8 |
| 4-Hydroxybenzoic acid | 0 |
| Nicotinic acid | 0 |
| p-Coumaric acid | 0 |
| Caffeic acid | 0 |
| Cinnamic acid | 0 |
| Vanallic acid | 0 |
| Indole-3-acetic acid | 0 |
| 2,4-Dichlorophenoxyacetic acid | 0 |
| Jasmonic acid | 0 |
| Gibberellic acid | 0 |

^{a}Values are averages of three independent measurements. All substrates were tested at 1 mM concentration. The activity of the recombinant GmSAMT1 with salicylic acid was set arbitrarily at 100%.

Increases in reaction rate by increasing concentrations of SAM and SA were found to obey Michaelis-Menten kinetics. Among the 14 examined substrates, SA, benzoic acid, anthranilic acid and 3-hydroxybenzoic acid served as in vitro substrates in the reactions catalyzed by the recombinant protein encoded by Glyma02g06070, although they showed obvious differences in activities. At substrate concentrations of 1 mM, the recombinant protein showed the highest activity with SA (100%) rather than benzoic acid (16.9%), anthranilic acid (9.3%) and 3-hydroxybenzoic acid (1.8%). For other substrates including JA, indole-3-acetic acid, and gibberellic acid, no activity was detected under the same reaction conditions. Glyma02g06070 catalyzed the methylation of SA with an apparent Km value of 32.9±1.7 μM. Final values represent the average of three independent measurements. Due to SA serving as its sufficient substrate, Glyma02g06070 was designated as GmSAMT1.

Biochemical Properties of Soybean SAMT:

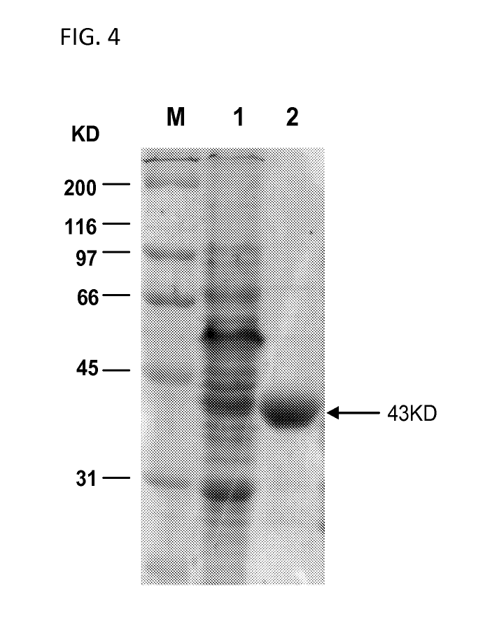

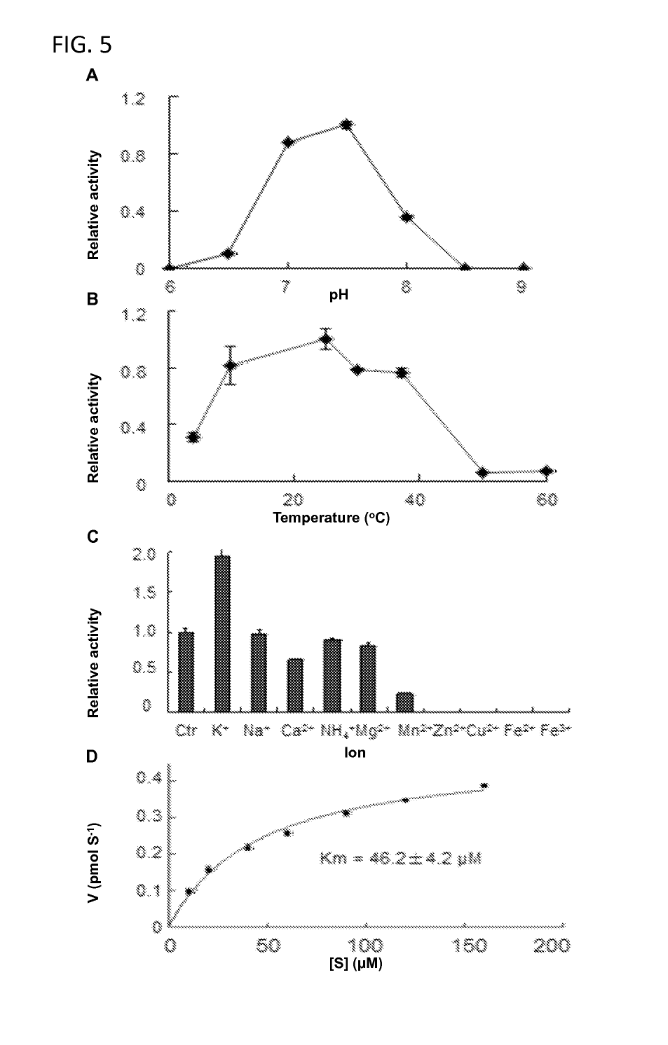

Recombinant GmSAMT1 with a His-tag expressed in E. coli was purified to electrophoretic homogeneity and subjected to detailed biochemical characterization. The molecular mass of the GmSAMT1 with His-tag protein estimated on the gel was 43 kDa, which was close to the expected size (FIG. 4). Purified recombinant GmSAMT1 exhibited an apparent Km value of 32.9±1.7 μM for salicylic acid. When enzyme assays were performed in buffers of different pH values, GmSAMT1 showed the highest level of catalytic activity at pH 7.5. (FIG. 5A). The optimal temperature for GmSAMT1 activity was 25° C. (FIG. 5B). The effects of various metal ions on GmSAMT1 activity were measured. Compared with control, GmSAMT1 exhibited double activity in presence of K while it showed no activity in presence of Cu²⁺, Zn²⁺, Fe²⁺ and Fe³⁺. GmSAMT1 activity was moderately inhibited by Mn²⁺ and Ca²⁺. Other metal ions, including Na⁺, NH₄⁺, and Mg²⁺, had minimal effect on GmSAMT1 activity (FIG. 5C). GmSAMT1 displayed pseudo Michaelis-Menton kinetics, with apparent Km value of 46.2±4.2 μM for salicylic acid (FIG. 5D). All the above results suggested the recombinant GmSAMT 1 showed salicylic acid methyltransferase activity. Taken together, the phylogenetic and biochemical evidence suggested that GmSAMT1 is a conserved enzyme among a number of plant species.

Reporter Gene Expression Through Tobacco Transient Transformation:

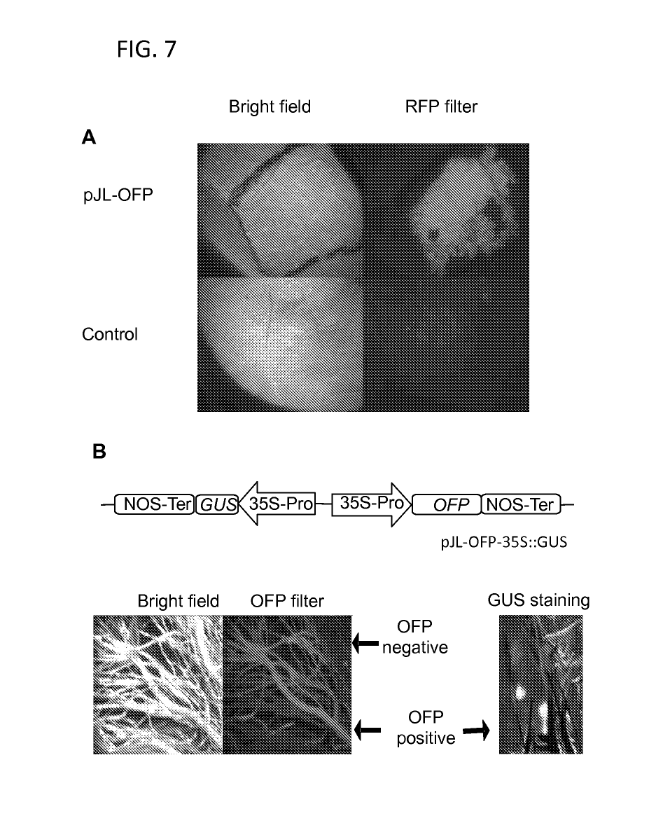

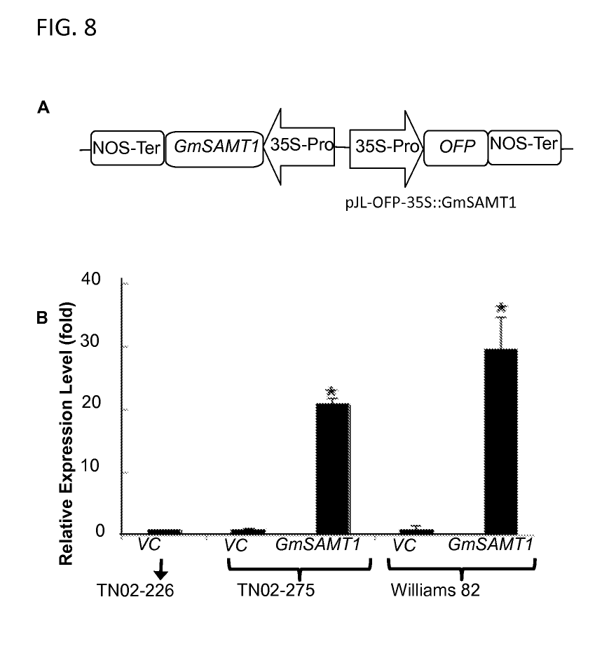

Before performing A. rhizogenes-mediated transformation, transient expression of pJL-OFP-35S::GUS in tobacco leaf through A. tumefaciens-mediated transformation was conducted to check OFP signal under OFP filter. Transgenic tobacco leaves harboring the target gene displayed OFP fluorescence, while the mock plant showed no OFP signal (FIG. 7A). This result helped to rapidly confirm the OFP expression.

After A. rhizogenes-mediated transformed soybean hairy roots with target genes were generated to be about 10 cm long, transgenic hairy roots harboring over-expressed GmSAMT1 were screened under OFP filter This method was demonstrated by checking GUS expression by staining the transgenic hairy roots harboring plasmid pJL-OFP-35S::GUS. All the positive transgenic hairy roots screened by OFP signal showed uniform GUS expression in the root tissue, while negative control hairy roots screened by OFP signal did not show GUS expression (FIG. 7B).

Gene Expression of GmSAMT1 and Several Pathogenesis-Related (PR) Genes:

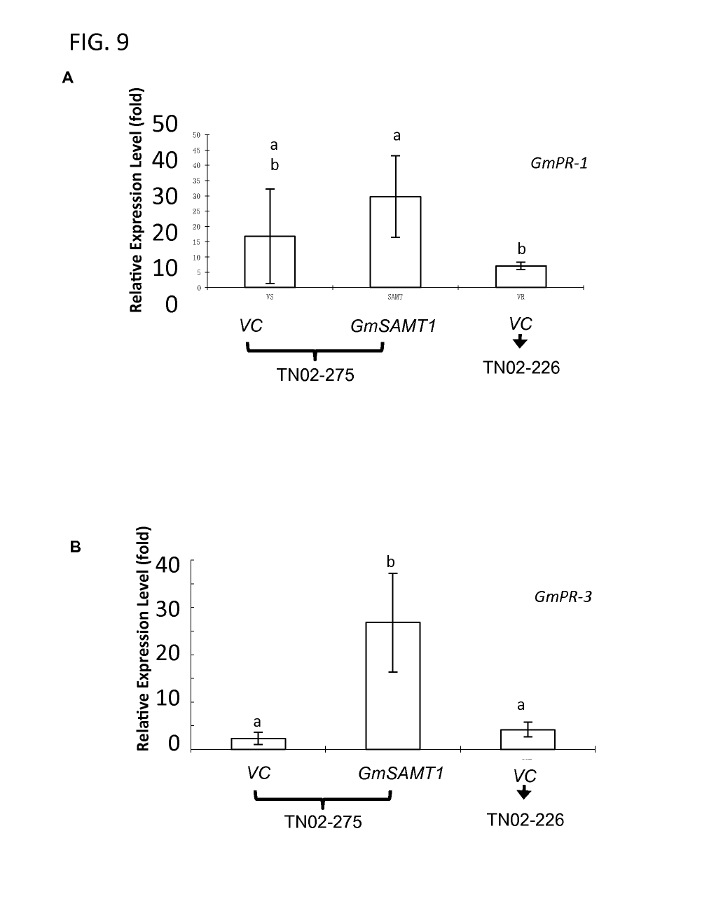

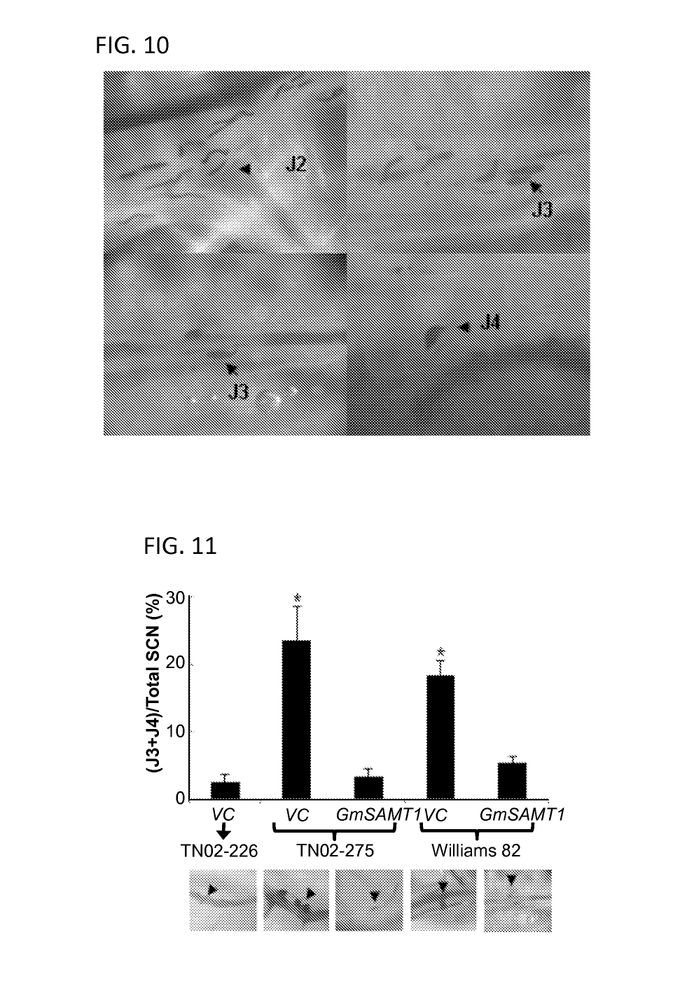

The gene expression level of GmSAMT1 and selected PR genes in transgenic hairy roots were examined by qRT-PCR (FIG. 8 and FIG. 9). The expression level of GmSAMT1 in over-expressed GmSAMT1 transgenic hairy roots was about 25 times higher than the control plants (FIG. 8B). GmPR-1 showed significant higher expression in over-expressed GmSAMT1 transgenic hairy roots compared with some control hairy root lines. But there was no significant difference of the gene expression of GmPR-1 between the transgenic hairy roots with over-expression of GmSAMT1 and vector control in the susceptible soybean background (FIG. 9A). This may suggest that the susceptible soybean can respond to exogenous proteins when SCN infection does not occur. When SCN parasitism happens, the defense system of the susceptible soybean may be changed or inhibited by SCNs, which produce and secrete parasitism proteins into soybean to aid themselves in the suppression of host plant defense. Chorismate mutase was found to be one of the SCN parasitism proteins, which can manipulate the plant's shikimate pathway. Since the SA biosynthesis is closely associated with the shikimate pathway, the SA signaling pathway of the susceptible soybean would be suppressed by SCN. It was already found that a local suppression of SA signaling in Arabidopsis roots was required for beet cyst nematode parasitism. In future, the expression of PR genes needs to be examined for the SCN-infected transgenic hairy roots. Since PR-1 gene was considered as a marker gene of SA signaling pathway, over-expressed GmSAMT1 might increase the signal transduction of SA pathway by providing abundant mobile molecule MeSA for responding SCN infection. Interestingly, the expression of PR-3, which encodes soybean chitinase, was observed to be higher in over-expressed GmSAMT1 transgenic hairy roots than other control lines (FIG. 9B). JA was found to induce PR-3 expression in Arabidopsis.

SCN is the most economically damaging pest of soybean in the U.S., so the mechanism of the interaction between soybean and SCN has been studied to better control this pest. With the development of genomics, a number of microarray analyses have been performed to study gene expression changes of soybean when infected by SCN. However, the various experiments are not congruent owing to different types of soybean and SCN samples. In the present study, the candidate gene was identified based on microarray analysis of two genetically-related soybean sister lines TN02-226 and TN02-275, which are resistant and susceptible, respectively, to the SCN race 2 infection.

During SCNs' inoculation, GmSAMT1 activity may also be manipulated through changing the concentration of potassium of soybean cells. The most common aboveground symptom of SCN infection is a potassium deficiency symptom with a bright yellowing of the margins of leaves. However, such SCN-induced potassium deficiency symptoms are the result of the plant's inability to raise sufficient potassium due to nematode feeding. It was demonstrated that potassium concentration in soybean roots grown at the medium level of potassium fertility condition was decreased by SCN (Smith et al. 2001). The result indicated that in vitro GmSAMT1 showed double activity in the presence of potassium, which suggests that SCN may also inhibit the activity of GmSAMT1 by decreasing the concentration of potassium in addition to suppressing the transcriptional level of GmSAMT1.

GmSAMT1 may have a role in crosstalk between SA and JA signaling pathway in soybean resistance against SCN. It was demonstrated that the gene expression of rice SAMT can be induced by JA. MeSA was also detected to be highly elevated after the wild-type Arabidopsis was treated by MeJA. As disclosed herein, the over-expression of GmSAMT1 in the susceptible soybean line caused up-regulation of GmPR-1 and GmPR-3, which has the integrative effect of SA and JA signaling pathways. Microarray analyses also indicate that the JA pathway was activated in the soybean defense response against SCN. Therefore, the SA-dependent pathway, combined with JA pathway through the regulatory points, including NPR1, WRKY family transcription factor, and LOX, play a role of restraining the development of SCN. The results presented herein indicated that GmSAMT1 is another regulatory point in the crosstalk between SA and JA pathways.

Resistance to SCN:

The resistance of soybean to SCN has been assessed by assaying SCN development, i.e., nematodes, cysts, or eggs per plant. In this study, this method was modified according to Melito et al., BMC Plant Biology 10:104, 2010. The ratio of the number of J3 and J4 stage nematodes to the total SCN number was set as the index, (J3+J4)/total SCN, to evaluate the resistance among the different hairy roots two weeks after inoculation with SCN eggs.

Female nematodes from J2 to J4 stages were observed for most transgenic hairy root sections and male nematodes were occasionally observed (FIG. 11). The number of SCN varied widely among individual transgenic and control plants. The mean number of established SCN was not significantly different between the transgenic hairy root line and the controls (data not shown). However, the ratio, (J3+J4)/total SCN, showed a significant difference between transgenic hairy roots and susceptible control line. The (J3+J4)/total SCN ratios for the transgenic hairy roots overexpressing GmSAMT1 (3.4% for TN02-275, 5.3% for Williams 82) were comparable to the resistant vector control (2.5% for TN02-226), and were significantly different with the susceptible vector control (23.6% for TN02-275, 18.4% for Williams 82). The transgenic soybean hairy roots with overexpressing GmSAMT1 had significantly fewer female juvenile SCNs developing to J3 and J4 stages compared with vector control in the susceptible soybean background. When the susceptibility level of vector control in the susceptible background was arbitrarily set at 100%, the transgenic soybean hairy roots overexpressing GmSAMT1 has significant reduction of susceptibility (85.6% reduction for TN02-275, 71.2% reduction for Williams 82).

Of most interest is that transgenic hairy roots with overexpressing GmSAMT1 exhibit higher resistance to SCN than control transgenic hairy roots (FIG. 11). The resistance mediated by GmSAMT1 is not line-specific, because two susceptible lines showed the same phenotype (FIG. 11). The biological role of GmSAMT1 in SCN resistance can be further evaluated using stable transformation.

Expression of SA Pathway Related Genes in Transgenic Hairy Roots:

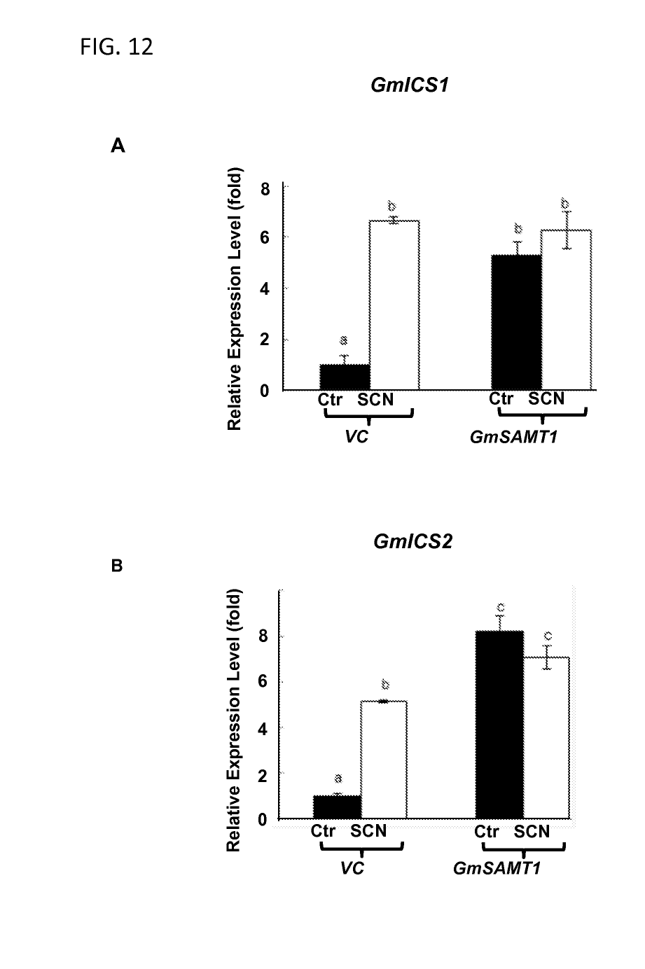

Transcript abundance for an SA-biosynthesis protein, isochorismate synthase (ICS)-encoding gene was assayed in SAMT-transgenic hairy roots (FIG. 12). In Arabidopsis, AtICS1 was required for the pathogen-induced accumulation of SA. Although ICS gene was proposed to be a critical gene for SA biosynthesis, little is known about the role of ICS in soybean. The expression of GmICS1 (Glyma01g25690) (FIG. 12A) and GmICS2 (Glyma03g17420) (FIG. 12B) was examined by qRT-PCR as well. Without SCN treatment, both GmICS1 and GmICS2 in the GmSAMT1 overexpression transgenic hairy roots showed higher transcript abundance (5.3 times and 8.2 times) than controls. Under SCN challenge treatment there was no significant difference on the expression of GmICS1 between the transgenic hairy root and control line. For GmICS2, the transgenic hairy roots had slightly greater transcript abundance than controls.

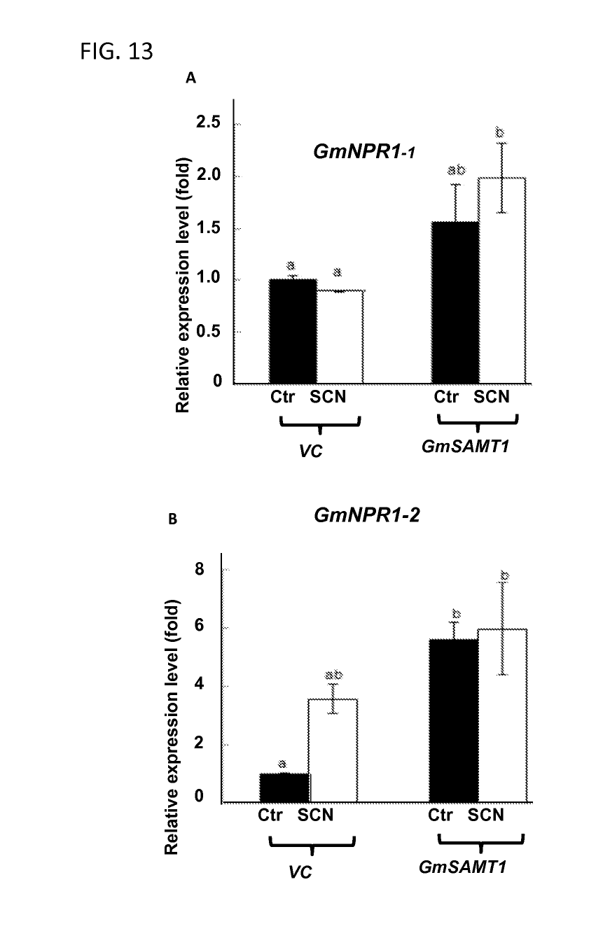

To further explore the SA dependent signal transduction pathway, the key regulatory gene of the SA signal pathway, two homolog of non-expressor of PR1 (NPR1) genes, GmNPR1-1 (Glyma09g02430) and GmNPR1-2 (Glyma15g13320) in soybean were tested by qRT-PCR (FIG. 13). For GmNPR1-1, there was no significant difference on the expression level between the transgenic hairy root and control lines without SCN treatment. However, with SCN treatment, GmNPR1-1 showed higher expression than the control (FIG. 13A). For GmNPR1-2, there was no significant difference in the expression level between the transgenic hairy root and control line with SCN treatment. However, without SCN treatment, GmNPR1-2 showed higher expression than controls. Taken together, GmNPR1-2 showed relatively higher expression in the transgenic hairy root in response to SCN treatment (FIG. 13B).

Biological Roles of GmSAMT1 in Soybean Defense Against Soybean Mosaic Virus:

To test whether GmSAMT1 is also involved in resistance against SMV, qRT-PCR was also conducted on measuring the expression of GmSAMT1 and GmPR-1 during interaction between SMV and soybean. In this assay two soybean lines L78-379 (Rsv1) and Williams 82 (rsv1) were utilized. Soybean genotype L78-379 is an isoline of Williams, but contains the Rsv1 resistance gene from PI 96983 (Rsv1). Inoculation of L78-379 (Rsv1) with a mutant derived from SMV-N(N25) results in induction of delayed hypersensitive response, whereas infection of Williams 82 (rsv1) with the same virus causes mosaic.

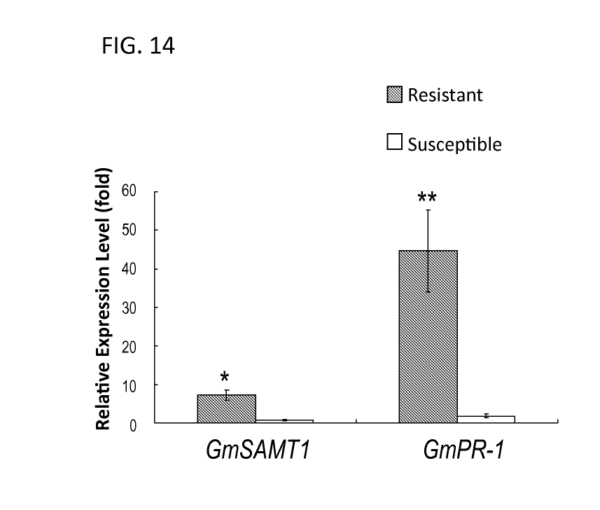

When the resistant response occurred for L78 against soybean virus 25 (21 days post inoculation), GmSAMT1 expression of the soybean top leaves is around 7.3 times that in the mock plant, and interestingly, a high expression level (44.6 times) of GmPR-1 were also observed. While in the susceptible response of soybean Williams 82, the expression of GmSAMT1 and GmPR-1 was reduced to half of that in the mock plant (FIG. 14). So the strongly positive correlation between the expression of GmSAMT1 and GmPR-1 indicates that GmSAMT1 plays a role in soybean defense against soybean mosaic virus.

Taken together, the gene expression differences of GmSAMT1 and selected PR genes, were up-regulated in the two inoculated tissues of the resistant soybean line, roots and leaves but down-regulated or not changed in the susceptible soybean line, demonstrates soybean responses against pathogens.

### Example 2

### Isolation and Characterization of GmSABP2-1

Plant, Nematode, Bacteria, and Chemical Sources:

In this study, two genetically-related soybean lines, TN02-226 and TN02-275, were used for gene cloning and generating soybean hairy roots. The breeding details for these two soybean lines and have been described. SCN race 2 eggs were used as the pathogens in the bioassay of the hairy roots, with the same inoculation method described above. Escherichia coli strain BL21 (DE3) CODONPLUS® was used for expressing the recombinant protein. A. rhizogenes strain K599 was utilized for carrying the over-expressed GmSABP2-1 and generating soybean hairy roots. The chemicals used in this study were obtained from Sigma-Aldrich.

Database Search and Sequence Analysis:

Both the sequences of Affymetrix probes GmaAffx.92649.1.S1_at and Gma.5867.1.A1_s_at showed 100% similarity with one soybean gene, Glyma16g26060. Based on the annotation in phytozome (as available on the world wide web at phytozome.net/soybean), this gene encodes an α/β fold hydrolase. Alignment of Glyma16g26060 with two other characterized methyl ester esterase was performed to study the conserved domains of this candidate protein.

Isolation of Glyma16g26060 Gene from Soybean:

Total RNA extraction and DNA contamination removal were performed as described. To further study the function of Glyma16g26060, the full length cDNA sequence of Glyma16g26060 was obtained from public soybean database (as available on the world wide web at phytozome.net/soybean). Using the specific primers ATGGGTTCACAAAATTGTATGGATAGG (SEQ ID NO: 15) and TCATGCATATTTAGTCGCTATCTGCTG (SEQ ID NO: 16), thermal cycling conditions were: 94° C. for 2 min followed by 35 cycles at 94° C. for 30 s, 57° C. for 30 s and 72° C. for 1 min, and a final extension at 72° C. for 10 min. The PCR product was cloned into vector pEXP5/CT-TOPO®. The sequencing result of the PCR product from TN02-226 soybean was consistent with that predicted in the soybean database on Williams 82 soybean.

To verify its biochemical function, the candidate gene was then cloned into a protein purification vector, pET100/D-TOPO® vector and expressed in the E. coli strain BL21 (DE3) CODONPLUS®. Protein expression was induced by isopropyl β-D-1-thiogalactopyranoside (IPTG) at a concentration of 500 μM for 18 h at 25° C., with cells lysed by sonication. E. coli-expressed Glyma16g26060 with a His-tag was purified from the E. coli cell lysate using Ni-NTA agarose following the manufacturer instructions (Invitrogen). Protein purity was verified by SDS-PAGE and protein concentrations were determined by the Bradford assay.

Methyl Ester Esterase Activity Assay:

A two-step radiochemical esterase assay was performed to determine the activity of E. coli-expressed Glyma16g26060 following a protocol previously reported by (Forouhar et al., Proc. Natl. Acad. Sci. USA 102: 1773-1778, 2005). The reaction of the first step was the reaction of substrate 10 μM MeSA and purified recombinant E. coli-expressed Glyma16g26060 for 30 min at 25° C. After that, the sample was boiled to stop the reaction and denature the enzyme. The second reaction step was started with the addition of 3 μM ¹⁴C labeled S-adenosyl-L-methionine (SAM) with a specific activity of 51.4 mCi/mmol (Perkin Elmer, Boston, Mass., USA), and respective known purified methyltransferase with high activity. Here, GmSAMT1 mentioned previously was used for SA methyltransferase enzyme. The reaction was preceded for 30 min at 25° C. Then radiolabeled products were extracted and the radioactivity was counted using a liquid scintillation counter.

Determination of Kinetic Parameters of Soybean SABP2:

The increase in reaction rate with increasing concentrations of MeSA was evaluated with the radiochemical assay described above and was found to obey Michaelis-Mention kinetics. Appropriate enzyme concentrations and incubation time were determined in time-course assays so that the reaction velocity was linear during the assay period. To determine the Km for MeSA, the concentrations of MeSA were independently varied in the range from 5 μM to 100 μM. Lineweaver-Burk plots were made to obtain apparent Km value. Optimal pH for GmSABP2-1 was assayed from pH 6.0 to pH 9.0. Final values are an average of three independent measurements.

Evaluation of SCN Resistance of Transgenic Hairy Roots with Over-Expression of GmSABP2-1:

Transgenic soybean hairy roots with over-expressed GmSABP2-1 were utilized to evaluate the biological function of GmSABP2-1. The steps including creating the construct with over-expressed GmSABP2-1 and OFP reporter gene, generating the transgenic soybean hairy roots harboring over-expressed GmSABP2-1, the SCN inoculation, and demographics assay of nematodes were performed as described, but GmSABP2-1 gene was used in place of GmSAMT1 gene.

Statistical Analysis;

The resistance difference between soybean hairy roots with over-expressing GmSABP2-1 and control soybean hairy roots was analyzed by one way ANOVA using the mixed model of SAS (SAS 9.2 version), and least squares means of the resistance index were separated using Fisher's LSD test. A P<0.05 was considered significant.

### Results

Identification and Sequence Analysis of One Putative SCN-Resistant Gene in Soybean:

Microarray analysis revealed genes showing significant expression changes during resistant and susceptible reactions to SCN. One of these genes was significantly up-regulated only in SCN inoculated resistant soybean roots whereas it was significantly down-regulated in SCN inoculated susceptible soybean roots through the microarray analysis and quantitative real time PCR.

To obtain the complete open reading frames of this resistance related gene, the sequences of Affymetrix SoyChip probe GmaAffx.92649.1.S1_at and Gma.5867.1.A1_s_at which identified the target gene in microarray analysis, were used as a query to BLAST search against the soybean database GmGDB/GMtranscript (as available on the world wide web at plantgdb.org/GmGDB/cgi-bin/blastGDB.p1), soybean gene Glyma16g26060 was found to show the highest similarity (100%) with the sequence of the above two probes and predicted to belong to α/β hydrolase gene superfamily.

Glyma16g26060 Encodes Soybean Methyl Ester Esterase:

A BLAST search revealed that Glyma16g26060 is 61.2% identical to the previously characterized NtSABP2 (GenBank accession AAR87711.1) at the amino acid sequence level, and its identity with the NtSABP2 is the highest among the soybean homolog genes. The alignment of peptide sequences among Glyma16g26060 and NtSABP2, At4g37150 was performed (FIG. 15). Glyma16g26060 contains the catalytic triad (Ser-86, His-239, and Asp-211) that is highly conserved among SABP2 orthologs from tobacco, Arabidopsis (AtMES), poplar and potato (StMES1) as well as the closely related members of the α/β hydrolase superfamily. Glyma16g26060 also contains all the 15 amino acids identified in NtSABP2 that interacts with SA. In contrast, AtMES9 contains only 5 and StMES1 shares 12 of these 15 residues. The high sequence similarity and conservation of critical SA-binding residues between Glyma16g26060 and NtSABP2 indicted that these two proteins shared similar biochemical properties. To confirm the prediction from the databases, the full-length cDNA of the Glyma16g26060 was cloned via reverse-transcription polymerase chain reaction (RT-PCR) from SCN-infected root tissue of soybean TN02-226 line and sequenced. The sequenced gene contained 786 bp, encoding a protein of 261 amino acids.

Biochemical Properties of Soybean SABP2:

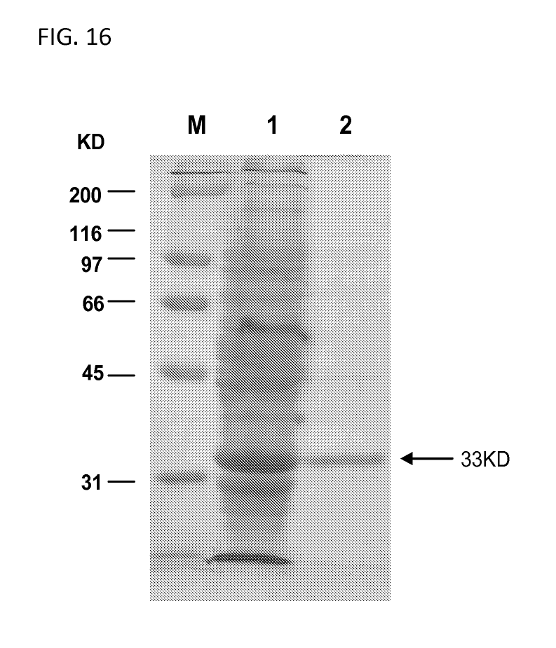

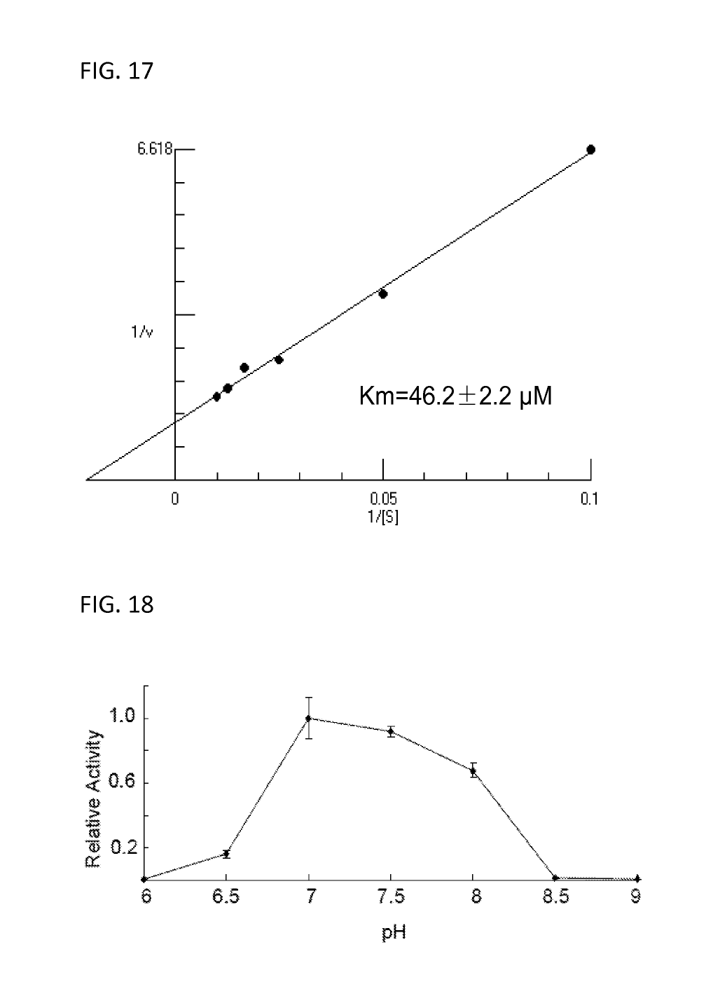

To further explore the biochemical properties of the enzyme encoded by Glyma16g26060, this gene was cloned into a protein expression vector. Recombinant Glyma16g26060 with a His-tag expressed in E. coli was purified to electrophoretic homogeneity and subjected to detailed biochemical characterization. The molecular mass of the Glyma16g26060 with His-tag protein estimated on the gel was 33 kDa (FIG. 16), which was close to the expected size of our target enzyme. Purified recombinant Glyma16g26060 showed the activity of converting MeSA to SA. Under steady-state conditions, Glyma16g26060 hydrolyzed MeSA with an apparent Km value of 46.2±2.2 μM (FIG. 17). So Glyma16g26060 was designated as soybean SABP2 (GmSABP2-1). When enzyme assays were performed in buffers with different pH conditions, GmSABP2-1 showed the highest level of catalytic activity at around pH 7.0 (FIG. 18).

Biological Role of Soybean SABP2 in SCN Resistance:

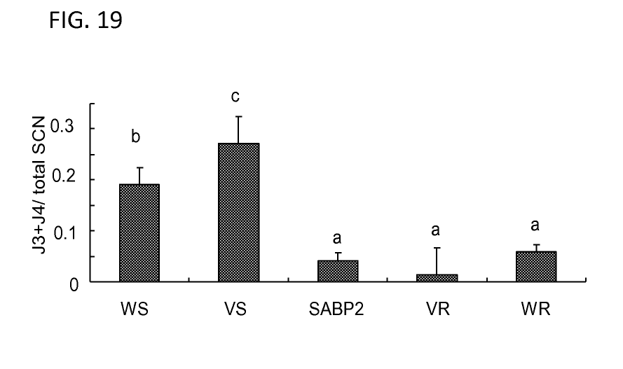

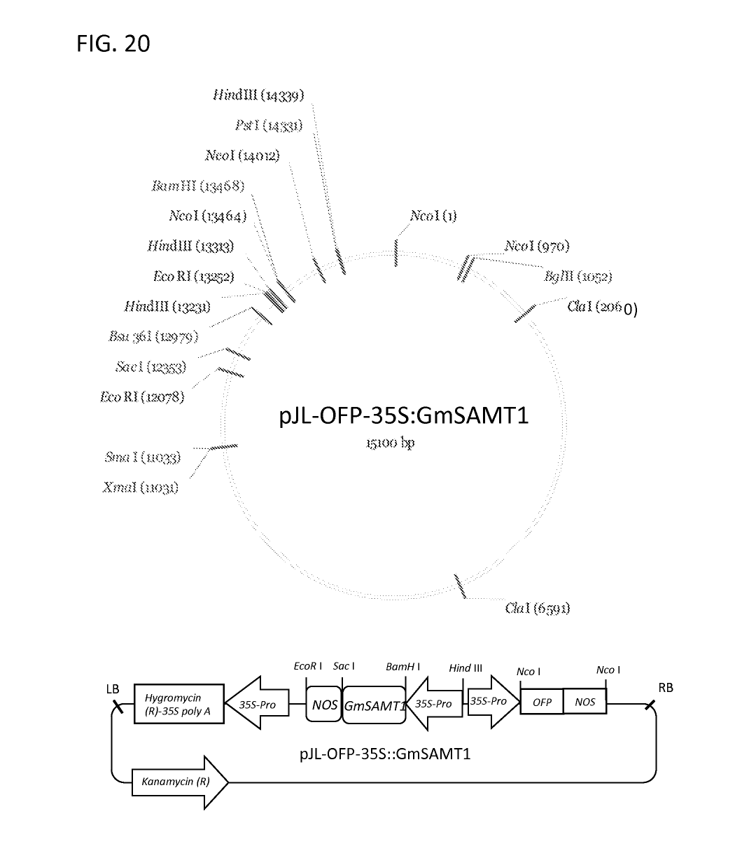

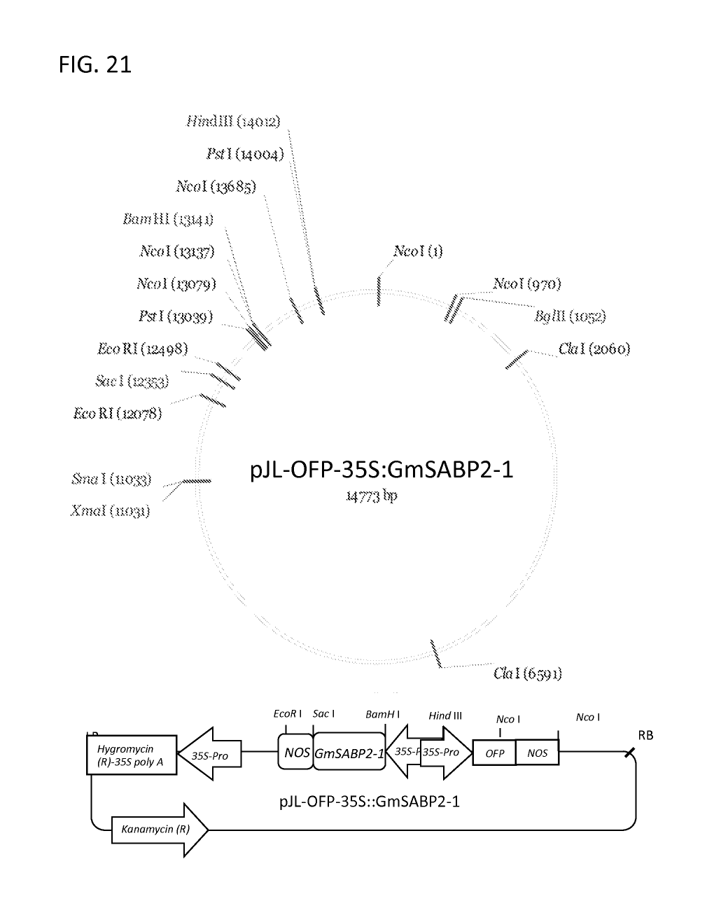

The study of the biological role of SABP2 in other plant species was performed through observing the leaves' resistance under virus or bacteria infection. Since the soybean resistance against SCN occurs in the root tissue, the number of SCNs that exist in the transgenic hairy roots at different stages were recorded to analyze the resistance level and evaluate the biological function of GmSABP2-1. As mentioned, the different resistant responses to SCN race 2 between the susceptible (TN02-275) and resistant (TN02-226) soybean lines occurred on the J3 stage of SCN, the time point after two-week inoculation by SCNs was chosen for analyzing the demographics of SCN in all the hairy roots with respective constructs. The index of J3+J4/total SCN number was used to estimate the resistance between the susceptible line with over-expressed GmSABP2-1 and other control lines, the same with the assay described. The susceptibility level of vector control in the susceptible background was arbitrarily set at 100%. The results showed the transgenic soybean hairy roots with over-expressed GmSABP2-1 had significantly fewer female SCN juvenile developed to J3 stage, compared with vector control line in the susceptible soybean background (84.5% reduction). And there was no significant difference on the resistance level between the transgenic hairy roots with over-expressed GmSABP2-1 and vector control in the resistant soybean background, which also had significant fewer female SCN juvenile developed to J3 stage, compared with negative vector control in the susceptible soybean background (94.8% reduction) (FIG. 19).

Described is the identification of GmSABP2-1 from soybean and demonstrated that it shared high similarity and conserved motifs with its tobacco and Arabidopsis orthologs. Through further biochemical assay, it was indicated that recombinant GmSABP2-1 exhibited esterase activity toward MeSA. The Km of GmSABP2-1 (46.2 μM) was higher than the Km of NtSABP2 (8.6 μM) PtSABP2-2 (24.6 μM), and lower than the Km of AtMES1 (57.3 μM), AtMES9 (147.1 μM), PtSABP2-1(68.2 μM), and StMES1 (57.9 μM). The differences in these kinetic parameters may reflect endogenous MeSA levels of plant species.

The transgenic hairy roots with over-expressed GmSABP2-1 in the susceptible soybean line, showed increased resistance against SCN. This result indicted that a certain expression level of GmSABP2-1 was required for soybean defense against SCN. GmSABP2-1 is an important component of receiving MeSA signal to induce SAR in soybean defense against SCN, considering the temporal and spatial differences of SCN on reaching the feeding sites of soybean vascular tissues within the SCN populations. The increased level of GmSABP2-1 protein by up-regulating its gene expression under SCN infection may benefit for capture of the mobile MeSA. Demethylation of MeSA catalyzed by GmSABP2-1 may be able to increase the SA level in the distal undamaged soybean root tissues since its ortholog NtSABP2 has the role of inducing SAR in the undamaged tobacco leaf.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, chemical moieties, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims.

1. An isolated nucleic acid molecule comprising a nucleic acid sequence that encodes a pest resistance protein at least 80% identical to a protein encoded by the nucleic acid sequence according to SEQ ID NO: 1 or SEQ ID NO: 2 or a degenerate variant thereof or a functional fragment thereof.

2. An isolated nucleic acid molecule comprising a promoter operably linked to the isolated nucleic acid molecule of claim 1.

3. An expression vector comprising the nucleic acid molecule of claim 2.

4. The vector of claim 3, wherein the vector is pJL-OFP-35S::GmSAMT1 (SEQ ID NO: 3) or pJL-OFP-35S::GmSABP2-1 (SEQ ID NO: 4).

5. An isolated host cell transformed with the vector of claim 3.

6. A construct comprising isolated nucleic acid molecule claim 1 operably linked to a promoter.

7. The construct of claim 6, wherein the construct confers an agronomic trait to a plant in which it is expressed.

8. The construct of claim 7, wherein the agronomic trait comprises pest resistance.

9. The construct of claim 8, wherein pest resistance comprises resistance to soybean cyst nematode (Heterodera glycines, SCN).

10. A transgenic plant stably transformed with the construct of claim 6.

11. A seed of the transgenic plant of claim 10, wherein the seed comprises the construct.

12. The transgenic plant of claim 10, which plant is a soybean plant.

13. A method of producing a transgenic plant comprising transforming a plant cell or tissue with the construct of claim 6.

14. A method of enhancing pest resistance in a plant comprising transforming a plant cell or tissue with the construct of claim 6, thereby enhancing pest resistance in the plant.

15. The method of claim 13, wherein the plant is a dicotyledon or a monocotyledon.

16. The method of claim 14, wherein the plant is a monocotyledon or a dicotyledon.

17. The method of claim 14, wherein pest resistance comprises resistance to soybean cyst nematode (Heterodera glycines, SCN).

18. The transgenic plant of claim 14, which plant is a soybean plant.

19. A plant cell or tissue transformed with the construct of claim 6.

20. The plant cell or tissue of claim 19, wherein the plant cell or tissue is from a dicotyledon.

21. The plant cell or tissue of claim 19, wherein the plant cell or tissue is derived from a monocotyledon.

22. A plant cell, fruit, leaf, root, shoot, flower, seed, cutting and other reproductive material useful in sexual or asexual propagation, progeny plants inclusive of F1 hybrids, male-sterile plants and all other plants and plant products derivable from the transgenic plant of claim 10.

23. A method of producing a commodity plant product, comprising obtaining the plant of 10 or a part thereof, wherein the commodity plant product is protein concentrate, protein isolate, soybean hulls, meal, flour or oil and producing the commodity plant product therefrom.
